(19) United States
(12) Patent Application Publication
(10) Pub. No.: US 2016/0274130 A1
(43) Pub. Date: Sep. 22, 2016

(54) Antibody Compositions and Immunoassay Methods to Detect Isoforms of Anti-Müllerian Hormone

(71) Applicant: ANSH LABS LLC, Webster, TX (US)

(72) Inventors: Gopal V. SAVJANI, El Lago, TX (US); Ajay KUMAR, Friendswood, TX (US)

(21) Appl. No.: 14/888,739

(22) PCT Filed: Nov. 8, 2013
(86) PCT No.: PCT/US13/69172

§ 371 Date: Nov. 3, 2015

Related U.S. Application Data
(60) Provisional application No. 61/724,485, filed on Nov. 9, 2012.
(60) Provisional application No. 61/835,420, filed on Jun. 14, 2013.
(60) Provisional application No. 61/889,868, filed on Oct. 11, 2013.

(51) Int. Cl. G01N 33/74; C07K 16/26

(52) U.S. Cl. CPC G01N 33/74; C07K 16/26; G01N 2333/575

(57) ABSTRACT

Disclosed are compositions and methods for detecting and quantifying human anti-Mtillerian hormone (AMH) in biological samples. In particular, the invention provides novel methods of measuring different forms of AMH in a biological sample, such as human plasma The anti-AMH antibody compositions disclosed herein enable reproducible measurement and quantitation of AMH, including dimeric forms of the AMH protein and fragments thereof. The antibody compositions disclosed herein find particular utility as diagnostic tools for single epitope sandwich-based AMH assays, which can be used to diagnose a variety of medical conditions.

### BACKGROUND

### Anti-Müllerian Hormone

Anti-Müllerian Hormone (AMH), also referred to as Müllerian inhibiting factor (MIF), Müllerian-inhibiting hormone (MIH), and Müllerian-inhibiting substance (MIS), is a 140-kilodalton (kDa) dimeric glycoprotein hormone, that is part of the transforming growth factor-β (TGF-β) superfamily (Zec et al., 2011). All members of this superfamily are dimeric glycoproteins that are variously involved in the regulation of tissue growth and/or cell differentiation. Like other members of the TGF-β superfamily, AMH protein is synthesized as a large precursor having a short 18 amino acid signal sequence, that is processed into a pre-prohormone that ultimately yields homodimers (Zec et al., 2011). Before secretion, the mature hormone undergoes glycosylation and dimerization resulting in a 140-kDa dimer of identical, 70-kDa, disulfide-linked, monomer subunits (Zec et al., 2011). Each monomer has an N-terminal domain (known as the “pro” region) and a C-terminal domain (known as the “mature” region) (Zec et al., 2011).

It is thought that AMH may require the N-terminal domain to accentuate the activity of the C-terminal domain to attain full bioactivity (Wilson et al., 1993). During cytoplasmic transit, AMH is cleaved at a specific site between the pro region and the mature region of the 70-kDa monomer to form two polypeptides: a 58-kDa pro region and a 12-kDa mature region (Zec et al., 2011). These two polypeptides remain non-covalently attached (Zec et al., 2011).

AMH plays an important role in sexual differentiation. AMH is produced by the Sertoli cells of the testis in the male, and by ovarian granulosa cells in the female. In males, AMH is responsible for the regression of the Müllerian ducts that would differentiate into the female oviducts, uterus and the upper portion of the vagina (Lee et al., 1993). In males, secretion of AMH by the Sertoli cells commences during embryogenesis and continues throughout life. From birth to puberty, AMH remains secreted at high levels. Upon the onset of puberty, AMH levels rapidly decrease, but its secretion is maintained at a constant level for most of the male life (Rajpert-De Meyts et al., 1999). In the female, serum AMH is maintained at relatively low levels compared to the male. AMH is expressed in ovarian granulosa cells and is present in females prior to birth, before menarche, and throughout the reproductive years (Hagen et al., 2010; Rajpert-De Meyts et al., 2009; Visser et al., 2005). When menstrual cycling begins, circulating AMH slowly decreases throughout life and becomes undetectable at menopause (Kumar et al., 2010).

Mammalian AMH polypeptides have been sequenced among a variety of species, and a homology alignment of the known sequences identifies 11 to 12 conserved cysteine residues across species (Lee et al., 1993). Seven of these conserved cysteine residues are located in the mature region of the protein (namely, at amino acid positions 462, 488, 492, 525, 526, 557, and 559, relative to the human AMH protein set forth in Genbank Accession number P03971). Thus, the mature region demonstrates the greatest degree of sequence homology between species, with 108 of the last 112 residues being conserved between the rat and human sequences (Lee et al., 1993).

### AMH Genetics

The gene encoding human AMH has been sequenced and isolated, and it is located on the short arm of chromosome 19 (Zec et al., 2011). The amino acid sequence of human AMH (SEQ ID NO:21, obtained from The Universal Protein Resource) can be found in FIG. 1.

### The Role of AMH in Mammalian Cell Function

Several clinical applications for measuring biological fluid AMH have been identified in humans. AMH's “popularity” as a biological marker is primarily due to its unique characteristics in gynecological endocrinology (Nelson et al., 2011). AMH is produced by granulosa cells in the ovary, allowing its use as a marker of granulosa cell function (Nelson et al., 2011). It is also the only hormonal marker for gonadotropin-independent folliculogenesis since it is secreted only by granulosa cells from primary to small antral follicles (Nelson et al., 2011). The measurement of AMH may help diagnose clinical conditions that include, but are not limited to: ovarian reserve in an IVF setting (Seifer et al., 2007; 2008), ovarian function, oocyte quality, premature ovarian failure, ovarian insufficiency, ovarian granulosa cell tumor (Long et al., 2000), breast cancer (Segev et al., 2000), ovarian function for childhood cancer survivors (Bath et al., 2003), polycystic ovary syndrome (Catteau-Jonard et al., 2008), menopause (Van Disseldorp et al., 2008; Broer et al., 2011) and intersex disorders (Rey et al., 1999).

### Diagnostic Assays for AMH

Several assays have previously been described for detecting one or more forms of AMH polypeptide. Hudson et al. (1990) developed an enzyme-linked immunoassay to detect AMH. It employs two monoclonal antibodies raised against human recombinant AMH, both directed to different epitopes in the pro region of the molecule. Use of this assay to measure AMH in humans from infancy to adulthood has been reported (Lee et al., 1996). The report found that AMH was stable when stored at −20° C. for up to 2 years, or up to three freeze-thaw cycles, but the measured values decreased by 50% or more after three freeze-thaw cycles. In addition, their report determined that the antibodies used in the Hudson assay did not recognize the mature region of the AMH protein. The antibodies were specific for full-length, unprocessed human AMH polypeptide and were subsequently shown to bind in the “pro” region of the polypeptide. The antibodies had a much lower affinity for the amino-terminal fragment (i.e., the “pro” region) than for the unprocessed intact protein (see, e.g., Lee et al, 1996).

A second AMH assay has been described that utilizes a pair of monoclonal antibodies, one binding to an epitope in the pro region, the other to an epitope in the mature region of human AMH (Long et al., 2000).

A third AMH assay has been described by Al-Qahtani et al., (2005) that employs two monoclonal antibodies, each of which binds to epitopes within the pro region of the protein.

A fourth AMH assay (exemplified in U.S. Pat. No. 7,897,350) employs two antibodies, each of which binds to distinct epitopes within the mature region of the AMH protein. While able to detect and quantify AMH in different biological samples (including mammalian samples such as human, monkey, mouse, rat, bovine and horse), the assay is limited in that in can only bind to the mature region of an AMH molecule. In a full length AMH molecule, the mature region is not exposed because of the seven cysteine inter and intra disulfide bonds (See seven cysteine residues in mature region of sequence, as shown above). The antibody epitopes are not always exposed for binding when AMH is in the full-length form. Thus, this assay fails to accurately measure AMH in a given sample.

A comparison of these four AMH assays is shown below in Table 1.

TABLE 1
Comparison of four prior AMH assays

| Antibody pair | Raised against | Epitopes | AMH Measurement | species |
|---|---|---|---|---|
| Hudson et al. 1996 | MAb anti-human | Pro region, do not recognize | Variability in AMH | hAMH, non-human |
| | rAMH | mature region. Greater | concentration reported due | primate. Does not |
| | | Immunoreactivity to full-length | to differences in the extent | recognize bovine |
| | | unprocessed hAMH (70 kD) over | of processing of the AMH | and rodent. |
| | | amino terminal fragment (pro | in vivo. | |
| | | region). | | |
| Long et. al 2000 | MAb anti-human | One mAb to pro region and other | The pro region of AMH is | hAMH. |
| | rAMH | to the mature region. | subject to proteolytic | |
| | | | cleavage during incubation | |
| | | | in solution | |
| Al-Qahtani et al. 2005 | MAb anti-human | Pro region, do not recognize | Variability in hAMH | hAMH. Do not |
| | rAMH | mature region. | reported. Capture is stable | recognize rat AMH. |
| | | | to proteolysis. Ab | |
| | | | recognizes both bands after | |
| | | | proteolytic cleavage. | |
| | | | Detection Ab does not | |
| | | | recognize the smaller | |
| | | | fragment after proteolytic | |
| | | | cleavage of the pro region | |
| | | | of hAMH. | |
| AMH Gen II ELISA | MAb anti-human | Both the mAb's recognizes the | Greater homology to other | Human, monkey, |
| 2009 | rAMH | mature region | species. Measurement | mouse, rat, bovine |
| | | | unaffected by proteolysis. | and horse. |
| | | | Assay not specific to AMH | |
| | | | fragments. Mature AMH | |
| | | | is embedded and | |
| | | | constrained due to seven | |
| | | | cystine inter and intra S—S | |
| | | | bonds. Epitopes not | |
| | | | exposed for binding in its | |
| | | | full length form. | |

### Deficiencies in the Prior Art

The three most popular commercially-available AMH assays in use today are the Diagnostic System Labs (DSL) AMH assay, the Immunotech assay (IOT) and the Beckman Coulter AMH Gen II assay (Gen II). These assays have different affinities for different forms of AMH found in circulation: native AMH, recombinant unprocessed AMH and processed AMH.

Unfortunately, none of the commercial assays presently available for AMH can distinguish between the monomeric and dimeric forms of the protein. The antibodies of each assay all bind to both forms of the protein, making them unsuitable for distinguishing between the two forms of AMH either in vitro, or in situ.

In fact, Nelson et al. (2011) elaborated on the particular weaknesses of the DSL assay and the IOT assays saying that they used different antibodies and different calibration materials, which resulted in substantially different results when the two assays were employed to quantify the protein under similar sample conditions.

According to a recent study, “the Gen II assay provides AMH results which are 20-40% lower than those measured using the DSL assay.” (Rustamov, et al., 2012). The study noted that the Gen II assay validation studies claimed that the assay gave AMH values approximately 40% higher than those found with the DSL assay (Rustamov, et al., 2012). The data suggested that AMH was not stable in some conditions, and such stability issues were more pronounced with the Gen II assay (Rustamov, et al., 2012). This variability in results is extremely troubling.

According to the Stages of Reproductive Aging Workshop (STRAW) committee, “lack of standardized assays for [AMH] remains an important limitation in efforts to stage reproductive aging and to translate research findings to cost effective tools. Given the importance of AMH in relation to fertility and its relative stability across the menstrual cycle, the development of an international standard for the assessment of AMH is of paramount importance” (Harlow et al., 2012).

Although it is not presently known what forms of AMH are clinically relevant, it is known that different forms of the protein exist in mammalian circulation, and different forms of the protein can be identified in various cells and tissues. There remains an unmet need in the art for developing an improved AMH detection assay that can identify and quantify specific isoforms of AMH protein. Moreover, there is a strong desire in the diagnostic arena for a single, standardized, facile, reproducible, and commercially-available assay (Nelson et al., 2011; Harlow et al, 2012, Rustamov et al., 2012).

### SUMMARY OF THE INVENTION

The present invention generally relates to immunological assays and methods for measuring compounds in biological samples. In particular, the invention provides novel methods of measuring different isoforms of anti-Müllerian hormone (AMH) and related polypeptides in a biological sample using a single epitope sandwich immunoassay. In addition, the invention provides a method to completely separate the full-length AMH and AMH related polypeptides into mature and pro-AMH fragments and the measurement of fragmented AMH isoforms or related polypeptides in sandwich immunoassays. These novel methods, employing novel anti-AMH antibodies enable reproducible measurements of AMH, and particularly dimeric isoforms of the AMH protein and its fragments in biological samples.

The present invention overcomes limitations in the prior art by providing compositions and methods for measuring AMH and AMH-derived fragments, and in particular, in biological samples. In particular, the invention provides methods of measuring dimeric AMH in a biological sample, and in particular, provides immunodetection-based assays that are superior to conventional AMH assays, which are unable to distinguish between dimeric and other forms (such as monomeric) of the molecule. A further advantage of the present invention is that the invention facilitates the measurement and quantitation of AMH isoforms (and particularly AMH dimeric isoforms), with a method that completely separates the full-length AMH and AMH related polypeptides into mature and pro AMH fragments, and measures the fragmented AMH isoforms or related polypeptides in sandwich immunoassay. In the present assay, proteolysis of the AMH does not affect the ability of the assay to accurately detect (and also quantify) all dimeric isoforms of AMH in the sample.

The present invention advantageously provides compositions and methods to fulfill the need of a highly-accurate, reproducible, and standardized commercial AMH assay. The present invention also provides methods for measuring biologically-active AMH in a sample, and evaluating its clinical potential. The present invention further advantageously provides compositions and methods for assessing and diagnosing several clinical conditions, including those involving expression, over-expression, under-expression, and/or absence of expression of AMH polypeptides in one or more selected cells, tissues, or biological fluids

One aspect of the invention is an isolated antibody that specifically binds to an epitope of human AMH contained in an amino acid sequence selected from the group consisting of: SEQ ID NO:106, SEQ ID NO:113, SEQ ID NO:150, SEQ ID NO:132, SEQ ID NO:129, SEQ ID NO:162, SEQ ID NO:163, SEQ ID NO:135, SEQ ID NO:152, SEQ ID NO:153, SEQ ID NO: 149, SEQ ID NO:138, SEQ ID NO:148, SEQ ID NO:173, SEQ ID NO:169, SEQ ID NO:170, SEQ ID NO:168, and SEQ ID NO:171. These sequences include epitopes in both the pro and mature regions of human AMH.

In related embodiments the isolated antibody specifically binds to an epitope of human AMH contained in an amino acid sequence selected from the group consisting of: SEQ ID NO:106, SEQ ID NO:113, SEQ ID NO:150, SEQ ID NO:132, SEQ ID NO:129, SEQ ID NO:162, SEQ ID NO:163, SEQ ID NO:135, SEQ ID NO:152, SEQ ID NO:153, SEQ ID NO: 149, SEQ ID NO:138, and SEQ ID NO:148. These sequences include epitopes in the pro region of human AMH.

In other related embodiments, the isolated antibody specifically binds to an epitope of human AMH contained in an amino acid sequence selected from the group consisting of: SEQ ID NO:173, SEQ ID NO:169, SEQ ID NO:170, SEQ ID NO:168, and SEQ ID NO:171. These sequences include epitopes in the mature region of human AMH.

Included as a related embodiment of the invention is a fragment of an antibody according to any of the embodiments above, the fragment selected from the group consisting of: Fv, Fab, F(ab′)₂, Fab′, dsFv, scFv, sc(Fv)₂, and diabody fragments. For example, the antibody is a dimer, trimer, a multimer, or includes a plurality of antigen-binding fragments thereof. For example, the antibody is a human, a humanized or a part-human antibody or an antigen-binding fragment thereof. For example, the antibody or the fragment thereof further includes a detectable label.

For example, the antibody is substantially free of binding to complement. For example, the antibody specifically binds to a linear epitope. For example, the antibody is an IgG antibody or an IgM antibody. For example, the antibody is a linear antibody, a chimeric antibody, a recombinant antibody, or any combination thereof.

Another aspect of the invention is a monoclonal antibody produced by hybridoma 33/A1 deposited as ATCC accession number PTA-13088. A related aspect of the invention is a monoclonal antibody produced by a hybridoma selected from the group consisting of 39/60, 39/38, and 39/48A.

A related embodiment of the invention is a recombinant vector including an isolated polynucleotide that encodes the antibody or the fragment thereof according to embodiments above, or a combination thereof. A further related embodiment of the invention is an isolated host cell, transfected with a nucleic acid segment that encodes the antibody or the fragment thereof, according to embodiments above, or a combination thereof.

Another aspect of the invention is an immunoconjugate including the antibody or the fragment thereof according to embodiments above, conjugated to a first diagnostic or a first therapeutic agent, or a combination thereof. In other related embodiments, the invention is a composition including: a) the immunoconjugate, and b) a physiologically-acceptable excipient, buffer, or diluent.

Still another aspect of the invention is an immunodetection reagent including the antibody or the fragment thereof according to embodiments above, linked to at least a first detectable label. For example, the first detectable label is selected from the group consisting of: a chemiluminescent agent, a colorimetric agent, an energy transfer agent, an enzyme, a fluorescent agent, a radioisotope, and any combination thereof. For example, the immunodetection reagent is bound to a solid support. For example, the solid support includes a protein binding surface selected from the group consisting of: a microtiter plate, a colloidal metal particle, an iron oxide particle, a latex particle, a polymeric bead, and any combination thereof.

Another aspect of the invention is a composition including: a) the antibody, or the fragment thereof, and b) a physiologically-acceptable excipient, buffer, or diluent.

Another aspect of the invention is an isolated murine hybridoma deposited as ATCC accession number PTA-13088.

Yet another aspect of the invention is a method of specifically detecting a dimeric form of human AMH, the method including: performing a sandwich ELISA assay using a first antibody for capture and a second antibody for detection, such that the first and second antibodies specifically bind to the same epitope of AMH, and such that the epitope is contained in an amino acid sequence selected from the group consisting of: SEQ ID NO:132, SEQ ID NO:152, and SEQ ID NO:106. For example, the first and the second anti-AMH antibodies are the same. For example, the first and the second antibodies are different.

Another aspect of the invention is a method of specifically detecting a monomeric form of human AMH, the method including: performing an ELISA assay using an antibody that specifically hinds to an epitope of AMH contained in an amino acid sequence selected from the group consisting of: SEQ ID NO:173, SEQ ID NO:106, SEQ ID NO:168, SEQ ID NO: 113.

Still another aspect of the invention is a method of quantifying a dimeric form of human AMH in a sample, the method including: performing a sandwich ELISA on the sample using a first antibody for capture and a second antibody for detection, such that the first and second antibodies specifically bind to the same epitope of AMH, and such that the epitope is contained in an amino acid sequence selected from the group consisting of: SEQ ID NO:132, SEQ ID NO:152, and SEQ ID NO:106; measuring a detection signal generated by an agent conjugated to the second antibody; and calculating the amount of dimeric human AMH in the sample by comparing the detection signal to a calibration curve correlating the amount of dimeric AMH to the detection signal. For example, each of the first and second antibodies does not substantially bind to complement. For example, dilution of the sample for quantification of AMH using the method yields substantially the same result, accounting for the dilution, when the sample is diluted to an AMH concentration anywhere in the range from about 20,000 pg/ml to about 1 pg/ml. For example, the quantified amount of dimeric human AMH in the sample corresponds to a sample concentration in the range from about 1 pg/ml to about 10 pg/ml.

Another aspect of the invention is a method of quantifying an amount of a pro-mature form of human AMH in a sample, the method including performing a sandwich ELISA assay on the sample using a pair of anti-AMH antibodies, such that one antibody of the pair binds to a first epitope within the pro region of human AMH and the other antibody of the pair binds to a second epitope within the mature region of human AMH, the first epitope contained in an amino acid sequence selected from the group consisting of: SEQ ID NO:106, SEQ ID NO:113, SEQ ID NO:150, SEQ ID NO:132, SEQ ID NO:129, SEQ ID NO:162, SEQ ID NO:163, SEQ ID NO:135, SEQ ID NO:152, SEQ ID NO:153, SEQ ID NO:149, SEQ ID NO:138, and SEQ ID NO:148; and the second epitope contained in an amino acid sequence selected from the group consisting of: SEQ ID NO:173, SEQ ID NO: 169, SEQ ID NO: 170, SEQ ID NO: 168, and SEQ ID NO: 171.

For example, one antibody of the pair specifically binds to an epitope contained in the amino acid sequence of SEQ ID NO:106 and the other antibody of the pair specifically binds to an epitope contained in the amino acid sequence of SEQ ID NO:171. For example, one antibody of the pair specifically binds to an epitope contained in the amino acid sequence of SEQ ID NO:152 and the other antibody of the pair specifically binds to an epitope contained in the amino acid sequence of SEQ NO:171 or the amino acid sequence of SEQ ID NO: 138, For example, the pro-mature form of human AMH is uncleaned. Alternatively, the pro-creature form of human AMH is cleaved into pro and mature fragments, and the pro and the mature fragments are associated in a non-covalent complex. For example, the dimeric pro-mature AMH is quantified.

Yet another aspect of the invention is a method for quantifying human AMH in a sample, the method including: performing a sandwich ELISA on the sample using a capture antibody and a detection antibody, such that the capture antibody specifically binds to a first epitope of human AMH and the detection antibody binds to a second epitope of human AMH such that the first and the second epitopes are contained in same or different amino acid sequences selected from the group consisting of: SEQ ID NO:106, SEQ ID NO:113, SEQ ID NO:150, SEQ ID NO:132, SEQ ID NO:129, SEQ ID NO:162, SEQ ID NO:163, SEQ ID NO:135, SEQ ID NO:152, SEQ ID NO:153, SEQ ID NO:149, SEQ ID NO:138, SEQ ID NO:148, SEQ ID NO:173, SEQ ID NO: 169, SEQ ID NO:170, SEQ ID NO:168, and SEQ ID NO:171.

For example, the first and the second epitopes are contained in different amino acid sequences selected from the group consisting of: SEQ ID NO:106 and SEQ ID NO:152, For example, the first and the second epitopes each are contained in the amino acid sequence of SEQ ID NO:106 For example, the first and the second epitopes each are contained in the amino acid sequence of SEQ II) NO:171, For example, each of the capture and detection antibodies does not substantially bind to complement.

Another aspect of the invention is a method of quantifying a pro fragment of human AMH in a biological sample, the method including: treating a portion of the sample with a detergent under conditions sufficient to dissociate a cleaved-reassociated isoform of human AMH into pro and mature fragments; quantifying an amount of a pro fragment of human AMH by performing a sandwich ELISA on the treated sample using a first antibody for capture and a second antibody for detection, such that the first and second antibodies specifically bind to the same or different epitopes in the pro portion of human AMH, and such that the epitopes are contained in an amino acid sequence selected from the group consisting of: SEQ ID NO:106, SEQ ID NO:113, SEQ ID NO:150, SEQ ID NO:132, SEQ ID NO:129, SEQ ID NO:162, SEQ ID NO:163, SEQ ID NO:135, SEQ ID NO:152, SEQ ID NO: 153, SEQ ID NO:149, SEQ ID NO:138, and SEQ ID NO:148; measuring a detection signal generated by an agent conjugated to the second antibody; and calculating the amount of pro fragment of human AMH in the sample by comparing the detection signal to a calibration curve correlating the amount of pro fragment of human AMH to the detection signal. For example, detergents used in the above embodiment may be selected from, for example: Triton® X-100, CHAPS (3-[3-cholamidopropyl) dimethylamino]-1-propanesulfonate, CHAPSO (3-(3-cholamidopropyl)-dimethyl-ammonio-2-hydroxy-1-propanesulfonate), deoxy-BIGCHAP (N,N-Bis-[3-(D-gluconamido)-propyl]-deoxycholamide), Tween® 20, sulfobetaine SB 12 (N-dodecyl-dimethyl-3-ammonio-1-propanesulfonate), sulfobetaine SB 14 (N-tetradecyl-dimethyl-3-ammonio-1-propanesulfonate), and sodium dodecyl sulfate (SDS). For example, SDS may be used in a concentration range of 0.1 to 3% in an ELISA assay.

For example, the first and second antibodies bind to the same epitope, and a dimeric pro fragment of human AMH is detected.

Further, an embodiment of the invention is a method of quantifying a mature fragment of human AMH in a biological sample, the method including: treating a portion of the sample with a detergent under conditions sufficient to dissociate a cleaved-reassociated isoform of human AMH into pro and mature fragments; quantifying an amount of a mature fragment of human AMH by performing a sandwich ELISA on the treated sample using a first antibody for capture and a second antibody for detection, such that the first and second antibodies specifically bind to the same or different epitopes in the mature portion of human AMH, and such that the epitopes are contained in an amino acid sequence selected from the group consisting of: SEQ ID NO:173, SEQ ID NO: 169, SEQ ID NO: 170, SEQ ID NO: 168, and SEQ ID NO: 171; measuring a detection signal generated by an agent conjugated to the second antibody; and calculating the amount of mature fragment of human AMH in the sample by comparing the detection signal to a calibration curve correlating the amount of pro fragment of human AMH to the detection signal.

For example, the first and second antibodies bind to the same epitope, and a dimeric mature fragment of human AMH is detected.

Another aspect of the invention is a method of quantifying an uncleaved pro-mature form of human AMH in a biological sample, the method including: treating a portion of the sample with a detergent under conditions sufficient to dissociate a cleaved-reassociated isoform of human AMH into pro and mature fragments; quantifying an amount of a pro-mature form of human AMH by performing a sandwich ELISA on the treated sample using a first antibody for capture and a second antibody for detection, such that the first antibody specifically binds to an epitope in the pro portion of human AMH, such that the epitope is contained in an amino acid sequence selected from the group consisting of: SEQ ID NO:106, SEQ ID NO:113, SEQ ID NO:150, SEQ ID NO:132, SEQ ID NO:129, SEQ ID NO:162, SEQ ID NO:163, SEQ ID NO:135, SEQ ID NO:152, SEQ ID NO: 153, SEQ ID NO: 149, SEQ ID NO: 138, and SEQ ID NO: 148; and the second antibody specifically binds to an epitope in the mature portion of human AMH, such that the epitope is contained in an amino acid sequence selected from the group consisting of: SEQ ID NO:173, SEQ ID NO:169, SEQ ID NO:170, SEQ ID NO:168, and SEQ ID NO:171; measuring a detection signal generated by an agent conjugated to the second antibody; and calculating the amount of pro-mature form of human AMH in the sample by comparing the detection signal to a calibration curve correlating the amount of pro-mature form of human AMH to the detection signal.

A further aspect of the invention is a method of quantifying an uncleaved pro-mature form of human AMH in a biological sample, the method including: treating a portion of the sample with a detergent under conditions sufficient to dissociate a cleaved-reassociated isoform of human AMH into pro and mature fragments; quantifying an amount of a pro-mature form of human AMH by performing a sandwich ELISA on the treated sample using a first antibody for capture and a second antibody for detection, such that the first antibody specifically binds to an epitope in the mature portion of human AMH, such that the epitope is contained in an amino acid sequence selected from the group consisting of: SEQ ID NO:173, SEQ ID NO:169, SEQ ID NO:170, SEQ ID NO:168, and SEQ ID NO:171; and the second antibody specifically binds to an epitope in the pro portion of human AMH, such that the epitope is contained in an amino acid sequence selected from the group consisting of SEQ ID NO:106, SEQ ID NO:113, SEQ ID NO:150, SEQ ID NO:132, SEQ ID NO:129, SEQ ID NO:162, SEQ ID NO:163, SEQ ID NO:135, SEQ ID NO:152, SEQ ID NO:153, SEQ ID NO:149, SEQ ID NO: 138, and SEQ ID NO:148; measuring a detection signal generated by an agent conjugated to the second antibody; and calculating the amount of pro-mature form of human AMH in the sample by comparing the detection signal to a calibration curve correlating the amount of pro-mature form of human AMH to the detection signal.

Still another aspect of the invention is a method of quantifying a cleaved reassociated pro-mature form of human AMH in a biological sample, the method including: (i) performing the method of quantifying an amount of a pro-mature form of human AMH according to embodiment above using a first portion of the sample that has not been detergent-treated and in which pro-mature forms of human AMH remain associated, whereby an amount of total (i.e., sum of cleaved and uncleaved pro-mature forms) pro-mature form of human AMH is determined; (ii) performing the method of quantifying an uncleaved pro-mature form of human AMH according to embodiment above using a second portion of the sample, whereby an amount of uncleaved pro-mature form of human AMH is determined; and (iii) subtracting the amount of uncleaved pro-mature form of human AMH determined in (ii) from the amount of total pro-mature form of human AMH determined in (i) to yield an amount of cleaved-reassociated pro-mature form of human AMH.

Another aspect of the invention is a method to aid in diagnosing or prognosing a disease or condition selected from the group consisting of: granulosa cell tumors, disorders of sex development, polycystic ovarian syndrome, gonadotoxicity; the method including quantifying AMH concentration in a patient sample using the isolated antibody and/or the fragment thereof according to embodiments above, and/or the method of detection or quantification according to any of the embodiments above. For example, the disorder of sex development is selected from conditions of newborns with atypical genitalia, conditions of adolescents presenting atypical sexual development, cryptorchidism, and atypical AMH production by the Sertoli cells of testes and its effects. For example, the gonadotoxicity is induced by chemotherapy.

Another aspect of the invention is a method of determining ovarian reserve in a female human subject, the method including quantifying AMH in a biological sample from the subject using the isolated antibody and/or the fragment thereof, and/or the method according to embodiments above. In related embodiments the method further includes comparing the quantified AMH to a standard that correlates AMH to a number of oocytes as a measure of ovarian reserve. For example, the sample is obtained prior to ovulation induction in the subject.

A further aspect of the invention is a method to aid in diagnosing ovarian insufficiency, the method including quantifying AMH in a biological sample using the isolated antibody according and/or the fragment thereof, and/or the method according to embodiments above.

Still another aspect of the invention is a method of predicting time to menopause in a female human subject, the method including quantifying AMH in a sample from the subject using the isolated antibody and/or the fragment thereof, and/or the method according to embodiments above.

Embodiments of the invention include use of the isolated antibody and/or the fragment thereof, according to the embodiments above in the manufacture of a diagnostic reagent or medicament. For example, the diagnostic reagent or medicament is for diagnosing, treating, preventing, or ameliorating the symptoms of an AMH-related disease or condition in a mammal. For example, the AMH-related disease or condition is selected from the group consisting of: primary ovarian insufficiency, peri-menopausal transition, polycystic ovarian syndrome, neonatal gender determination, cryptorchidism, testicular function, and precocious puberty.

Another aspect of the invention is an ELISA kit for quantification of human AMH, the kit including a pair of anti-AMH antibodies, the pair consisting of a capture antibody and a detection antibody, and instructions for use; such that the capture antibody and the detection antibody hind to a first and a second epitope, respectively; and such that each of the first and the second epitopes is contained in an amino acid sequence selected from the group consisting of: SEQ ID NO:106, SEQ ID NO:113, SEQ ID NO:150, SEQ ID NO:132, SEQ ID NO:129, SEQ ID NO:162, SEQ ID NO:163, SEQ ID NO:135, SEQ ID NO:152, SEQ ID NO:153, SEQ ID NO:149, SEQ ID NO:138, SEQ ID NO:148, SEQ ID NO:173, SEQ ID NO:169, SEQ ID NO:170, SEQ ID NO:168, and SEQ ID NO:171.

In related embodiments of the invention the capture and detection antibodies of the ELISA kit both bind the same epitope of dimeric human AMH, such that the epitope is contained in an amino acid sequence selected from the group consisting of: SEQ ID NO:132, SEQ ID NO:152, and SEQ ID NO:106. In other related embodiments, the kit further includes a streptavidin enzyme conjugate, and the detection antibody is conjugated to biotin. Related embodiments include a kit that further includes a set of known amounts of AMH for use as calibration standards. In related embodiments, the kit is used for determination of human AMH extracted from a dried blood sample. For example, the dried blood sample is in the form of dried blood adsorbed to a disc of an adsorbent material, e.g., a filter pater disc.

According to other related embodiments of the invention, dilution of a sample for quantification of AMH using the kit yields substantially the same result, accounting for the dilution, when the sample is diluted to an AMH concentration anywhere in the range from about 20,000 pg/ml to about 1 pg/ml. For example, the kit is suitable for quantifying an amount of dimeric human AMH present in a sample at a concentration in the range from about 1 pg/ml to about 10 pg/ml.

Yet another aspect of the invention is a method of inhibiting AMH from binding to an AMH receptor, the method including contacting a cell population having cells that express an AMH receptor with a composition including an amount of at least a first antibody according to any of claims 1-3, effective to substantially block the interaction of AMH with the AMH receptor.

Further, another aspect of the invention is a method of quantifying a cleaved-reassociated pro-mature form of human AMH in a biological sample, the method including: (i) performing the method of the embodiments above for quantifying the pro fragment or region of AMH, or the mature fragment or region of AMH, whereby an amount of total (i.e., sum of cleaved-reassociated and uncleaved pro-mature forms) pro-mature form of human AMH is determined; (ii) performing the method of the embodiments above for quantifying an amount of uncleaved pro-mature form of human AMH; and subtracting the amount of uncleaved pro-mature form of human AMH determined in (ii) from the amount of total pro-mature form of human AMH determined in (i) to yield an amount of cleaved-reassociated pro-mature form of human AMH.

Yet another aspect of the invention is a method of quantifying a fraction of uncleaved pro-mature form of human AMH in a biological sample, the method including: (i) performing the method of the embodiments above for quantifying the pro fragment or region of AMH, or the mature fragment or region of AMH, whereby an amount of total sum of cleaved-reassociated and uncleaved pro-mature forms) pro-mature form of human AMH is determined; performing the method of the embodiments above for quantifying an amount of uncleaved pro-mature form of human AMH; and (iii) determining a ratio of the amount determined in (ii) to the amount determined in (i) as a measure of the fraction of uncleaved pro-mature form of human AMH in the sample.

In one aspect, the invention provides an isolated, and preferably a purified, antibody (or one or more antigen binding fragments thereof, that binds, and preferably specifically binds to a mammalian (and preferably a human) AMH molecule, wherein the antibody or antigen binding fragment thereof includes at least one heavy chain variable region that comprises three complementarity-determining regions (CDRs) and at least one light chain variable region that comprises three CDRs, and wherein the antibody includes: (1) a variable light (VL) CDR1 that comprises, consists essentially of, or alternatively consists of a contiguous amino acid sequence from SEQ ID NO:3 or SEQ ID NO:13, a VL CDR2 that comprises, consists essentially of, or alternatively consists of a contiguous amino acid sequence from SEQ ID NO:4 or SEQ ID NO:14, a VL CDR3 that comprises, consists essentially of, or alternatively consists of a contiguous amino acid sequence from SEQ ID NO:5 or SEQ ID NO:15, a variable heavy (VH) CDR1 that comprises, consists essentially of, or alternatively consists of a contiguous amino acid sequence from SEQ ID NO:8 or SEQ ID NO:18, a VH CDR2 that comprises, consists essentially of, or alternatively consists of a contiguous amino acid sequence from SEQ ID NO:9 or SEQ ID NO:19, and a VH CDR3 that comprises, consists essentially of, or alternatively consists of a contiguous amino acid sequence from SEQ ID NO:10 or SEQ ID NO:20; (2) a light chain variable region having the amino acid sequence of SEQ ID NO:1 or SEQ ID NO:3, or a heavy chain variable region having the amino acid sequence of SEQ ID NO:2 or SEQ ID NO:4; or (3) a monoclonal antibody, designated herein as antibody #9 or antibody #10, the former of which is produced by a murine hybridoma splenic cell line designated 33/A1, and deposited with the ATCC under accession number PTA-13088.

In particular embodiments, the isolated antibody or antigen binding fragment thereof includes (a) a variable domain of heavy chain that includes a contiguous amino acid sequence from SEQ ID NO:7 or SEQ ID NO:17; and (b) a variable domain of light chain that includes a contiguous amino acid sequence from SEQ ID NO:2 or SEQ ID NO:12.

In certain embodiments, the antibodies of the present invention may be characterized as an scFv, a Fv, a Fab′, a Fab, a diabody, a linear antibody, a F(ab′)₂ antigen-binding fragment of an antibody, a chimeric antibody, a recombinant antibody, a dimer, a trimer, or a multimer of one or more AMH-specific antibodies. Preferably, such antibodies include human antibodies, humanized antibodies, part-human antibodies, diabodies, or one or more antigen-binding fragments or CDRs thereof.

In related embodiments, the invention provides a fragment of an AMH-specific monoclonal antibody, wherein the antibody specifically binds to a mammalian dimeric AMH molecule, but does not substantially bind to monomeric or other forms of the AMH molecule, and wherein the fragment includes: (a) a heavy chain having the amino acid sequence of SEQ ID NO:8 for CDR-1, the amino acid sequence of SEQ ID NO:9 for CDR-2 and the amino acid sequence of SEQ ID NO:10 for CDR-3; and (b) a light chain having the amino acid sequence of SEQ ID NO:3 for CDR-1, the amino acid sequence of SEQ ID NO:4 for CDR-2 and the amino acid sequence of SEQ ID NO:5 for CDR-3, wherein the fragment is selected in the group consisting of Fv, Fab, F(ab′)₂, Fab′, dsFv, scFv, sc(Fv)₂, diabody fragments, and any combination thereof.

For use in the manufacture and preparation of one or more immunodetection reagents, the antibodies and antigen binding fragments of the present invention preferably further include at least a first detectable label such as, for example, without limitation, a chemiluminescent agent, a colorimetric agent, an energy transfer agent, an enzyme, a fluorescent agent, a radioisotope, or any combination thereof, that is operably linked to the antibody or the antigen binding fragment.

The invention further provides isolated polynucleotides that include at least a first nucleic acid segment that encodes one or more of the antibodies or antigen binding fragments disclosed herein. Preferably the antibody or the antibody fragment is specific for a dimeric Anti-Mullerian Hormone, and preferably a dimeric human AMH molecule. In particular embodiment, such antibodies or antigen binding fragments thereof are encoded by at least a first nucleic acid sequence in accordance with any one of SEQ ID NO:1 to SEQ ID NO:4.

The invention further provides a recombinant vector that includes an isolated polynucleotide comprising an isolated nucleic acid segment that encodes one or more of the antibodies or a fragment thereof, as disclosed herein. Such vectors include, without limitation, plasmids, cosmids, phagemids, viral vectors, shuttle vectors, and the like.

The invention further provides an isolated host cell (preferably a mammalian host cell such as a human host cell) that is transformed by at least a first nucleic acid segment that encodes one or more of the antibodies or antigen binding fragments disclosed herein, or a combination thereof. Exemplary host cells include human, non-human primate, and murine cells, with the isolated murine hybridoma cell line (available on deposit with the ATCC as accession number PTA-13088) being particularly preferred.

The antibodies and antigen binding fragments of the present invention further include immunoconjugate compositions, in which antibody or the fragment is operably linked, coupled, covalently linked, or conjugated to a first diagnostic or a first therapeutic agent, or a combination thereof.

The invention also provides pharmaceutical and diagnostic compositions, and immunodetection reagents and the like that preferably include one or more of the dimeric AMH-specific antibodies or fragments thereof, or one or more of the AMH dimer-specific immunoconjugates formulated with one or more physiologically-acceptable excipients, buffers, diluents, or immunodetection assay systems.

The immunodetection reagents of the invention are preferably operably linked to at least a first detectable label, and may optionally be bound to a solid support such as a protein binding surface, including those selected from the group consisting of a microtiter plate, a colloidal metal particle, an iron oxide particle, a latex particle, a polymeric bead, and any combination thereof. In certain embodiments, the immunodetection reagents of the present invention may further optionally include a second, distinct detectable label operably attached thereto, or a second, distinct antibody operably linked thereto that has operably attached to it a second, distinct detectable label.

The invention also provides a method of detecting a mammalian dimeric AMH. This method, in an overall and general sense includes at least the step of contacting a composition suspected of containing a mammalian dimeric AMH with at least a first anti-AMH antibody, or antigen-binding fragment thereof, that binds to substantially the same epitope as the monoclonal antibody #9 (ATCC PTA-13088) or antibody #10, under conditions effective to allow the formation of AMH/antibody complexes and detecting the complexes so formed.

The invention further provides a method of inhibiting AMH from binding to an AMH receptor, and AMH substrate, an AMH enzyme, or an AMH-specific cell type, ligand, or any combination thereof. In an overall and general sense, such methods include at least the step of contacting a first cell population that includes one or more cells that are suspected of expressing an AMH receptor, or an AMH substrate, or an AMH enzyme, or AMH-specific cell type, ligand, or combination thereof, with a first composition that includes a biologically-effective amount of at least a first anti-AMH antibody that binds to substantially the same epitope as the monoclonal antibody #9 (ATCC PTA-13088) or antibody #10, or an antigen-binding fragment of the monoclonal antibody #9 (ATCC PTA-13088) or antibody #10.

The invention also provides a method of detecting a dimeric AMH molecule, without significantly detecting a monomeric or other form of an AMH molecule in a sample. Such methods generally involve at least the step of contacting a sample suspected of containing a population of monomeric, dimeric, and or other forms of an AMH molecule with at least a first composition that includes a biologically-effective amount of an anti-AMH dimer-specific antibody that binds to substantially the same epitope as the monoclonal antibody #9 (ATCC PTA-13088) or antibody #10, or an antigen-binding fragment of the monoclonal antibody #9 (ATCC PTA-13088) or antibody #10.

The invention further provides a method of quantifying the amount of mammalian dimeric AMH in a sample. Such methods generally involve at least the step of contacting a sample suspected of containing a population of mammalian dimeric AMH molecules with at least a first anti-AMH antibody, or antigen-binding fragment thereof, that binds to substantially the same epitope as the monoclonal antibody #9 (ATCC PTA-13088) or antibody #10, under conditions effective to allow the formation of AMH dimer/antibody complexes and detecting the complexes so formed.

The invention further provides an antibody that specifically binds to a dimeric form of a mammalian AMH molecule, but does not substantially bind to the monomeric form of a mammalian AMH molecule, and that comprises at least one complementarity-determining region (CDR) defined as: (a) a CDR of the monoclonal antibody #9 (produced by the hybridoma deposited as ATCC PTA-13088) or antibody #10; or (b) a CDR that has the amino acid sequence of SEQ ID NO:2 or SEQ ID NO:7, or a variant or mutagenized form of the amino acid sequence of SEQ ID NO:2 or SEQ ID NO:7, wherein the variant or mutagenized form maintains binding to the mammalian dimeric AMH molecule, and does not substantially bind to the monomeric form of a mammalian AMH molecule. In preferred embodiments, the antibody comprises at least one CDR of the monoclonal antibody #9 produced by the hybridoma deposited as ATCC PTA-13088, and more preferably, comprises at least one CDR from the variable regions of each of the heavy and light chains of monoclonal antibody #9 (ATCC PTA-13088).

In illustrative embodiments, the antibodies of the present invention may be used in diagnosis, in the manufacture of medicaments and/or diagnostics and/or immunodetection reagents, and preferably find utility in the quantitation, detection, diagnosis, prophylaxis, and/or therapy of one or more AMH-related diseases, disorders, abnormal conditions, dysfunctions, or one or more symptoms thereof in a mammal, and preferably in a human.

Such antibodies preferably comprise a variable domain having a human framework region, a human constant domain, or a combination thereof, and preferably have an affinity for dimeric mammalian AMH at least equal to the affinity of the monoclonal antibody #9 (ATCC PTA-13088) or antibody #10 for dimeric AMH, but do not substantially have an affinity for monomeric or other non-dimeric forms of a mammalian AMH molecule under similar physiological or experimental conditions.

In a further embodiment, the present invention provides a composition comprising an antibody pair that binds to the same epitope on an AMH homodimer, wherein one antibody binds to the epitope on one monomer of the AMH homodimer, and the other antibody binds to the identical epitope on the second monomer of the same AMH dimeric unit.

Another aspect of the present invention provides a composition comprising a single antibody pair that binds to the same epitope on Anti-Müllerian Hormone homodimer fragments, variants, analogs, agonist, chemical derivatives, functional derivatives or functional fragments of an AMH polypeptide, wherein one antibody binds to the epitope on one monomer of the AMH homodimer fragment, variant, analog, agonist, chemical derivative, functional derivative or functional fragment of an Anti-Müllerian Hormone polypeptide, and the second antibody binds to the identical epitope on the second monomer of the same AMH dimeric fragment, variant, analog, agonist, chemical derivative, functional derivative or functional fragment of an AMH dimeric polypeptide.

Another aspect provides a method for measuring an amount of Anti-Müllerian Hormone in a biological sample containing Anti-Müllerian Hormone using a single epitope sandwich immunoassay comprising: binding an antibody to an Anti-Müllerian Hormone homodimer, wherein the antibody binds to an epitope on one monomer of the Anti-Müllerian Hormone homodimer; binding a second antibody to the Anti-Müllerian Hormone, wherein the second antibody binds to the identical epitope on the second monomer of the same Anti-Müllerian Hormone dimeric unit, thereby creating an amount of bound second antibody; measuring the amount of bound second antibody; and calculating the amount of Anti-Müllerian Hormone in the sample.

In a further aspect, the invention provides a composition comprising a first and a second antibodies that each bind to the same first epitope on an AMH, wherein the first antibody binds to the first epitope on one monomer of the AMH homodimer, and the second antibody binds to the identical epitope on the second monomer of the same Anti-Müllerian Hormone dimeric unit.

The present invention also provides a composition comprising a first antibody and a second antibody, each of which bind to substantially the same epitopic amino acid sequence on two or more AMH dimeric fragments, variants, analogs, agonist, chemical derivatives, functional derivatives or on two or more functional fragments of an AMH polypeptide in a sample suspected of containing AMH molecules. Such compositions are particularly useful in a “single-epitope sandwich” (SES) immunoassay. In the assay, the first antibody binds substantially to a first epitope on one monomer of AMH homodimer fragments, variants, analogs, agonist, chemical derivatives, functional derivatives or functional fragments of an AMH polypeptide and the second antibody binds substantially to the identical epitope on the second monomer of the same AMH dimeric fragments, variants, analogs, agonist, chemical derivatives, functional derivatives or functional fragments thereof.

In a particular illustrative embodiment, the present invention provides a composition comprising an AMH antibody (designated herein as “antibody #9”) that is producible from a murine spleen hybridoma cell line (designated herein as “33/1A” which has been deposited with the American Type Culture Collection as accession number PTA-13088.

Polynucleotide sequences encoding the propeptide are shown in italics

Hybridoma B, antibody #9, Variable Light Chain
(SEQ ID NO: 1)
ATGGTGTCCACTTCTCAGCTCCTTGGACTTTTGCTTTTCTGGACTTCAGC
CTCCAGATGTGACATTATGATGACTCAGTCTCCAGCCACCCTGTCTGTGA
CTCCAGGAGATAGAGTCTCTCTTTCCTGCAGGGCCAGCCAGAGTATTAGC
GACTACTTACACTGGTATCAACAAAAATCACATGAGTCTCCAAGGCTTCT
CATCAAATATGCTTCCCAATCCATCTCTGGGATCCCCTCCAGGTTCAGTG
GCAGTGGATCAGGGTCAGATTTCACTCTCAGTATCAACAGTGTGGAACCT
GAAGATGTTGGAGTGTATTACTGTCAAAATGGTCACAGCTTTCCGCTCAC
GTTCGGAGCTGGGACCAAGCTGGAGCTGAAACGGGCTGATGCT

The encoded amino acid sequence is shown below:

(SEQ ID NO: 2)
Met Val Ser Thr Ser Gln Leu Leu Gly Leu Leu Leu
Phe Trp Thr Ser Ala Ser Arg Cys Asp Ile Met Met
Thr Gln Ser Pro Ala Thr Leu Ser Val Thr Pro Gly
Asp Arg Val Ser Leu Ser Cys Arg Ala Ser Gln Ser
Ile Ser Asp Tyr Leu His Trp Tyr Gln Gln Lys Ser
His Glu Ser Pro Arg Leu Leu Ile Lys Tyr Ala Ser
Gln Ser Ile Ser Gly Ile Pro Ser Arg Phe Ser Gly
Ser Gly Ser Gly Ser Asp Phe Thr Leu Ser Ile Asn
Ser Val Glu Pro Glu Asp Val Gly Val Tyr Tyr Cys
Gln Asn Gly His Ser Phe Pro Leu Thr Phe Gly Ala
Gly Thr Lys Leu Glu Leu Lys Arg Ala Asp Ala
Hybridoma B, antibody #9, Variable Light Chain
CDR1 amino acid sequence
(SEQ ID NO: 3)
Arg Ala Ser Gln Ser Ile Ser Asp Tyr Leu His
Hybridoma B, antibody #9, Variable Light Chain
CDR2 amino acid sequence
(SEQ ID NO: 4)
Tyr Ala Ser Gln Ser Ile Ser
Hybridoma B, antibody #9, Variable Light Chain
CDR3 amino acid sequence
(SEQ ID NO: 5)
Gln Asn Gly His Ser Phe Pro Leu Thr
Hybridoma B, antibody #9 Variable Heavy Chain
(SEQ ID NO: 6)
ATGGAATGGAGCTGGGTCTCTCTCTTCTTCCTGTCAGTAACTACAGGTG
TCCACTCCCAGGTTCAGCTGCAACAGTCTGACGCTGAGTTGGTGAAACC
TGGAGCTTCAGTGAAGATATCCTGCAAGGTTTCTGGCTACACCTTCACT
GACCATACTTTTCACTGGATGAAGCAGAGGCCTGAACAGGGCCTGGAAT
GGATTGGATATATTTATCCTAGAGATGGTAGTACTGATTACAATGAGAA
GTTCAAGGGCAAGGCCACATTGACTGCAGACAGATCCTCCAGCACAGTC
TACATGCAGCTCAACAGCCTGACATCTGAGGACTCTGCAGTTTATTTCT
GTGCAAGAGATCTCAGGCTTTCTTTTTCTTACTGGGGCCAAGGGACTCT
GGTCACTGTCTCTGCA

The encoded amino acid sequence is shown below:

(SEQ ID NO: 7)
Met Glu Trp Ser Trp Val Ser Leu Phe Phe Leu Ser
Val Thr Thr Gly Val His Ser Gln Val Gln Leu Gln
Gln Ser Asp Ala Glu Leu Val Lys Pro Gly Ala Ser
Val Lys Ile Ser Cys Lys Val Ser Gly Tyr Thr Phe
Thr Asp His Thr Phe His Trp Met Lys Gln Arg Pro
Glu Gln Gly Leu Glu Trp Ile Gly Tyr Ile Tyr Pro
Arg Asp Gly Ser Thr Asp Tyr Asn Glu Lys Phe Lys
Gly Lys Ala Thr Leu Thr Ala Asp Arg Ser Ser Ser
Thr Val Tyr Met Gln Leu Asn Ser Leu Thr Ser Glu
Asp Ser Ala Val Tyr Phe Cys Ala Arg Asp Leu Arg
Leu Ser Phe Ser Tyr Trp Gly Gln Gly Thr Leu Val
Thr Val Ser Ala
Hybridoma B, antibody #9, Variable Heavy
Chain CDR1 amino acid sequence
(SEQ ID NO: 8)
Asp His Thr Phe His
Hybridoma B, antibody #9, Variable Heavy
Chain CDR2 amino acid sequence
(SEQ ID NO: 9)
Tyr Ile Tyr Pro Arg Asp Gly Ser Thr Asp Tyr Asn
Glu Lys Phe Lys Gly
Hybridoma B, antibody #9, Variable Heavy Chain
CDR3 amino acid sequence
(SEQ ID NO: 10)
Asp Leu Arg Leu Ser Phe Ser Tyr

In a related illustrative embodiment, the present invention also provides a composition comprising an AMH antibody (designated herein as “antibody #10”) that is producible from a murine spleen hybridoma cell line (designated herein as “33/2A”).

Polynucleotide sequences encoding the pro-peptide are shown in italics:

Hybridoma A, antibody #10, Variable Light Chain
(SEQ ID NO: 11)
ATGATGTCCTCTGCTCAGTTCCTTGGTCTCCTGTTGCTCTGTTTTCAAGG
TACCAGATGTGATATCCAGATGACACAGACTACATCCTCCCTGTCTGCCT
CTCTGGGAGACAGAGTCACCATCAGTTGCAGGGCAAGTCAGGACATTAGC
AATTATTTAAACTGGTATCAGCAGAAACCAGATGGAACTGTTAAACTCCT
GATTTACTACACATCAAGATTACACTCAGGAGTCCCATCAAGGTTCAGTG
GCAGTGGGTCTGGAACAGATTATTCTCTCACCATTAGCAACCTGGAGCAA
GAAGATATTGCCACTTACTTTTGCCAACAGGGTAATACGCTTCCGTACAC
GTTCGGAGGGGGGACCAAGCTGGAAATAAAACGGGCTGATGCT

The encoded amino acid sequence is shown below:

(SEQ ID NO: 12)
Met Met Ser Ser Ala Gln Phe Leu Gly Leu Leu Leu
Leu Cys Phe Gln Gly Thr Arg Cys Asp Ile Gln Met
Thr Gln Thr Thr Ser Ser Leu Ser Ala Ser Leu Gly
Asp Arg Val Thr Ile Ser Cys Arg Ala Ser Gln Asp
Ile Ser Asn Tyr Leu Asn Trp Tyr Gln Gln Lys Pro
Asp Gly Thr Val Lys Leu Leu Ile Tyr Tyr Thr Ser
Arg Leu His Ser Gly Val Pro Ser Arg Phe Ser Gly
Ser Gly Ser Gly Thr Asp Tyr Ser Leu Thr Ile Ser
Asn Leu Glu Gln Glu Asp Ile Ala Thr Tyr Phe Cys
Gln Gln Gly Asn Thr Leu Pro Tyr Thr Phe Gly Gly
Gly Thr Lys Leu Glu Ile Lys Arg Ala
Hybridoma A, antibody #10, Variable Light Chain
CDR1 amino acid sequence
(SEQ ID NO: 13)
Arg Ala Ser Gln Asp Ile Ser Asn Tyr Leu Asn
Hybridoma A, antibody #10, Variable Light Chain
CDR2 amino acid sequence
(SEQ ID NO: 14)
Tyr Thr Ser Arg Leu His Ser
Hybridoma A, antibody #10, Variable Light Chain
CDR3 amino acid sequence
(SEQ ID NO: 15)
Gln Gln Gly Asn Thr Leu Pro Tyr Thr
Hybridoma A, antibody #10 Variable Heavy Chain
(SEQ ID NO: 16)
ATGGAATGGAGCTGGGTCTCTCTCTTCTTCCTGTCAGTAACTACAGGTGT
CCACTCCCAGGTTCAGCTGCAACAGTCTGACGCTGAGTCGGTGAAACCAG
GAGCTTCAGTGAAGATATCCTGCAAGGTTTCTGGCTACACCTTCACTGAC
CATACTATTCACTGGATGAAGCAGAGGCCTGAACAGGGCCTGGAATGGAT
TGGATATATTTATCCTAGAGATGGTAGTACTAACTACAATGAGAAGTTCA
AGGGCAAGGCCACATTGACTGCAGACAAATCCTCCAGCACAGCCTACATG
CAGCTCAACAGCCTGACATCTGAGGACTCTGCAGTCTATTTCTGTGCAAG
ATCATATTTGGATATGGACTACTGGGGTCAAGGAACCTCAGTCACCGTCT
CCTCA

The encoded amino acid sequence is shown below:

(SEQ ID NO: 17)
Met Glu Trp Ser Trp Val Ser Leu Phe Phe Leu Ser
Val Thr Thr Gly Val His Ser Gln Val Gln Leu Gln
Gln Ser Asp Ala Glu Ser Val Lys Pro Gly Ala Ser
Val Lys Ile Ser Cys Lys Val Ser Gly Tyr Thr Phe
Thr Asp His Thr Ile His Trp Met Lys Gln Arg Pro
Glu Gln Gly Leu Glu Trp Ile Gly Tyr Ile Tyr Pro
Arg Asp Gly Ser Thr Asn Tyr Asn Glu Lys Phe Lys
Gly Lys Ala Thr Leu Thr Ala Asp Lys Ser Ser Ser
Thr Ala Tyr Met Gln Leu Asn Ser Leu Thr Ser Glu
Asp Ser Ala Val Tyr Phe Cys Ala Arg Ser Tyr Leu
Asp Met Asp Tyr Trp Gly Gln Gly Thr Ser Val Thr
Val Ser Ser
Hybridoma A, antibody #10, Variable Heavy Chain
CDR1 amino acid sequence
(SEQ ID NO: 18)
Asp His Thr Ile His
Hybridoma A, antibody #10, Variable Heavy Chain
CDR2 amino acid sequence
(SEQ ID NO: 19)
Tyr Ile Tyr Pro Arg Asp Gly Ser Thr Asn Tyr Asn
Glu Lys Phe Lys Gly
Hybridoma A, antibody #10, Variable Heavy Chain
CDR2 amino acid sequence
(SEQ ID NO: 20)
Ser Tyr Leu Asp Met Asp Tyr

Another embodiment provides a method for measuring an amount of Anti-Müllerian Hormone homodimer fragments, variants, analogs, agonist, chemical derivatives, functional derivatives or functional fragments of an Anti-Müllerian Hormone polypeptide in a biological sample containing Anti-Müllerian Hormone using a single epitope sandwich immunoassay comprising: binding an antibody to an Anti-Müllerian Hormone homodimer fragment, variant, analog, agonist, chemical derivative, functional derivative or functional fragment of an Anti-Müllerian Hormone polypeptide, wherein the antibody binds to an epitope on one monomer of the Anti-Mullerian Hormone homodimer fragment, variant, analog, agonist, chemical derivative, functional derivative or functional fragment of an Anti-Müllerian Hormone polypeptide; binding a second antibody to the Anti-Müllerian Hormone homodimer fragment, variant, analog, agonist, chemical derivative, functional derivative or functional fragment of an Anti-Müllerian Hormone polypeptide, wherein the second antibody binds to the identical epitope on the second monomer of the same Anti-Müllerian Hormone homodimer fragment, variant, analog, agonist, chemical derivative, functional derivative or functional fragment of an Anti-Müllerian Hormone polypeptide, thereby creating an amount of bound second antibody; measuring the amount of bound second antibody; and calculating the amount of Anti-Müllerian Hormone fragments, variants, analogs, agonist, chemical derivatives, functional derivatives or functional fragments of an Anti-Müllerian Hormone polypeptide in the sample.

The present invention provides a composition comprising two antibodies that bind to the same epitope on an Anti-Müllerian Hormone, wherein one antibody binds to the epitope on one monomer of the Anti-Müllerian Hormone homodimer, and the other antibody binding to the identical epitope on the second monomer of the same Anti-Müllerian Hormone dimeric unit. These antibodies are able to measure an amount of Anti-Müllerian Hormone in biological fluid. The antibodies may be monoclonal antibodies. The epitope may be located anywhere on the Anti-Müllerian Hormone molecule, with each antibody binding to identical epitopes found on each monomer of the homodimeric unit.

The present invention provides a composition comprising two antibodies that bind to the same epitope on an Anti-Müllerian Hormone fragment, wherein one antibody binds to the epitope on one monomer of the Anti-Müllerian Hormone homodimer fragment dimer, and the other antibody binding to the identical epitope on the second monomer of the same Anti-Müllerian Hormone fragment homodimer. These antibodies are able to measure an amount of Anti-Müllerian Hormone fragments in biological fluid. The antibodies may be monoclonal antibodies. The epitope must be located on the fragment to be identified, with each antibody binding to the identical epitope found on each monomer of the fragment homodimeric unit.

Another embodiment provides a method for measuring an amount of Anti-Müllerian Hormone in a biological sample containing Anti-Müllerian Hormone using a single epitope sandwich immunoassay comprising: binding an antibody to an Anti-Müllerian Hormone, wherein the antibody binds to an epitope on one monomer of the Anti-Müllerian Hormone homodimer; binding a second antibody to the Anti-Müllerian Hormone, wherein the second antibody binds to the identical epitope on the on the second monomer of the same Anti-Müllerian Hormone homodimer, thereby creating an amount of bound second antibody; measuring the amount of bound second antibody; and calculating the amount of Anti-Müllerian Hormone in the sample.

Another embodiment provides a method for measuring an amount of Anti-Müllerian Hormone fragments, variants, analogs, agonist, chemical derivatives, functional derivatives or functional fragments of an Anti-Müllerian Hormone polypeptide in a biological sample containing Anti-Müllerian Hormone using a single epitope sandwich immunoassay comprising: binding an antibody to an Anti-Müllerian Hormone homodimer fragment, variant, analog, agonist, chemical derivative, functional derivative or functional fragment of an Anti-Müllerian Hormone polypeptide, wherein the antibody binds to an epitope on one monomer of the Anti-Mullerian Hormone homodimer fragment, variant, analog, agonist, chemical derivative, functional derivative or functional fragment of an Anti-Müllerian Hormone polypeptide dimer; binding a second antibody to the Anti-Müllerian Hormone homodimer fragment, variant, analog, agonist, chemical derivative, functional derivative or functional fragment of an Anti-Müllerian Hormone polypeptide, wherein the second antibody binds to the identical epitope on the second monomer of the Anti-Müllerian Hormone homodimer fragment variant, analog, agonist, chemical derivative, functional derivative or functional fragment of an Anti-Müllerian Hormone polypeptide homodimer, thereby creating an amount of bound second antibody; measuring the amount of bound second antibody; and calculating the amount of Anti-Müllerian Hormone homodimer fragments, variants, analogs, agonist, chemical derivatives, functional derivatives or functional fragments of an Anti-Müllerian Hormone polypeptide in the sample.

Another embodiment of the present invention describes a method for measuring isoforms of AMH comprising: an in situ method to separate the full-length AMH into mature and pro fragments, binding an antibody to a fragment, wherein the antibody binds to an epitope on the fragment, binding a second antibody to the fragment, wherein the second antibody binds to a second epitope on the fragment, thereby creating an amount of bound second antibody; measuring the amount of second antibody; and calculating the amount of fragments in the sample.

Another embodiment provides a method of measuring Anti-Müllerian Hormone in a biological sample in order to assess or diagnose a clinical condition. Clinical conditions include, but are not limited to, ovarian reserve, ovarian function, oocyte quality, oocyte quantity, premature ovarian failure, ovarian insufficiency, ovarian granulosa cell tumor, breast cancer, ovarian function for childhood cancer survivors, polycystic ovary syndrome, menopause and intersex disorders. Such method comprises: obtaining a serum sample from a subject; measuring the amount of AMH is said sample by binding an antibody to an Anti-Müllerian Hormone, wherein the antibody binds to an epitope on one monomer of the Anti-Müllerian Hormone homodimer; binding a second antibody to the Anti-Müllerian Hormone, wherein the second antibody binds to the identical epitope on the on the second monomer of the same Anti-Müllerian Hormone homodimer, thereby creating an amount of bound second antibody; measuring the amount of bound second antibody; calculating the amount of Anti-Müllerian Hormone in the sample; and using the AMH calculation to assess or diagnose said clinical condition.

Another embodiment provides a method of measuring Anti-Müllerian Hormone in a biological sample; combining the measurement of Anti-Müllerian Hormone with levels of other biomarkers, including but not limited to, Inhibin B and FSH; and using the combination of biomarkers to assess a clinical condition. Such method comprises: obtaining a serum sample from a subject; measuring the amount of AMH is said sample by binding an antibody to an Anti-Müllerian Hormone, wherein the antibody binds to an epitope on one monomer of the Anti-Müllerian Hormone homodimer; binding a second antibody to the Anti-Müllerian Hormone, wherein the second antibody binds to the identical epitope on the on the second monomer of the same Anti-Müllerian Hormone homodimer, thereby creating an amount of bound second antibody; measuring the amount of bound second antibody; calculating the amount of Anti-Müllerian Hormone in the sample; measuring the levels of a secondary biomarker in the sample, comparing the AMH measurement to the secondary biomarker measurement, and using the comparison to assess or diagnose said clinical condition.

Another embodiment provides a method of measuring Anti-Müllerian Hormone either alone, or in combination with other biomarkers in a biological sample, with an algorithm in order to assess a clinical condition.

The single epitope sandwich (“SES”) immunoassay in the present invention differs from all other commercially-available sandwich AMH assays. The first antibody (the “capture” antibody) and the second antibody (the “detection” antibody) both recognize substantially the same epitope on both monomers of the mature the AMH homodimer (or of one or more processed dimeric fragments of an AMH molecule). Thus, instead of using conventional methods in which two separate antibodies are required to perform the assay, in the present system, a single antibody can be employed to fulfill the role of both the “capture” and the “detection” antibodies. This one-antibody, dual same-site epitope detection-based SES immunoassay is a first among the commercially-available AMH assay systems. Importantly, the accuracy of this single antibody, same-epitope immunoassay is not affected by proteolytic fragmentation of AMH. Thus, this assay is the first AMH assay available that can detect and accurately measure the amount of true dimeric AMH in a sample.

### BRIEF DESCRIPTION OF THE DRAWINGS

For promoting an understanding of the principles of the invention, reference will now be made to the embodiments, or examples, illustrated in the drawings and specific language will be used to describe the same. It will, nevertheless be understood that no limitation of the scope of the invention is thereby intended. Any alterations and further modifications in the described embodiments, and any further applications of the principles of the invention as described herein are contemplated as would normally occur to one of ordinary skill in the art to which the invention relates.

The following drawings form part of the present specification and are included to demonstrate certain aspects of the present invention. The invention may be better understood by reference to the following description taken in conjunction with the accompanying drawings, in which like reference numerals identify like elements.

FIG. 1 shows the amino acid sequence of human AMH (SEQ ID NO:21).

FIG. 2 depicts the instability of the full-length AMH molecule. AMH is prone to proteolysis between the pro and mature subunits; adapted from U.S. Pat. No. 5,359,033.

FIG. 3 shows the cleavage sites of human AMH. The AMH molecule is prone to proteolysis between the pro and mature regions. The pro region has an additional cleavage site; from U.S. Pat. No. 7,897,350.

FIG. 4 illustrates the current AMH assays on the market, which will fail to detect some cleaved fragments of AMH, because the antibody epitopes may be located on two opposite sides of the cleavage site.

FIG. 5A shows the current AMH assay on the market, which are able to detect both dimeric and monomeric forms of AMH.

FIG. 5B depicts how the assay in current invention will not recognize monomeric AMH, because the capture and the detection antibody are directed to the same epitope on each side of the AMH dimer. The current invention will only detect true dimeric AMH.

FIG. 6 demonstrates the fact that different forms of AMH exist in circulation. The experiment used different antibody pairs to detect AMH in the same samples under the same conditions. Each antibody pair measured different amounts of AMH in the same samples. The antibody pairs are believed to be measuring different isoforms of AMH in the same samples, leading to different measurements of AMH.

FIG. 7 shows the antibody screening via detection of cell supernatants on AMH coated plates.

FIG. 8 shows an example of a sandwich assay claimed in the current invention. As AMH concentration increase, the optical density (O.D.) level increases.

FIG. 9 demonstrates the comparative signal to noise ratio (S/N) of antibody pairs with the epitopes in the pro region of the human AMH related protein. The ratio is determined by dividing the total signal divided by the signal at a blank level. The antibody pairs with the highest ratios are better antibody pairs, because the can measure AMH at a higher level and distinguish AMH from the noise. All of these antibody pairs were tested under identical conditions.

FIG. 10 demonstrates the comparative signal to noise ratio (S/N) of antibody pairs with the capture antibody epitope on the Pro region and the detection antibody on the mature region of the human AMH related protein. The ratio is determined by dividing the total signal divided by the signal at a blank level. The antibody pairs with the highest ratios are better antibody pairs, because the can measure AMH at a higher level and distinguish AMH from the noise. All of these antibody pairs were tested under identical conditions.

FIG. 11 demonstrates the comparative signal to noise ratio (S/N) of antibody pairs with the capture antibody epitope on the mature region and the detection antibody on the pro region of the human AMH related protein. The ratio is determined by dividing the total signal divided by the signal at a blank level. The antibody pairs with the highest ratios are better antibody pairs, because the can measure AMH at a higher level and distinguish AMH from the noise. All of these antibody pairs were tested under identical conditions.

FIG. 12 demonstrates the comparative signal to noise ratio (S/N) of antibody pairs with the epitopes in the mature region of the human AMH related protein. The ratio is determined by dividing the total signal divided by the signal at a blank level. The antibody pairs with the highest ratios are better antibody pairs, because the can measure AMH at a higher level and distinguish AMH from the noise. All of these antibody pairs were tested under identical conditions.

FIG. 13 is a table demonstrating the calibration of a claimed AMH sandwich assay. STD1-STD5 are calibrator samples with concentration levels from 0-10 ng. The actual concentration of each sample can be found in the fifth column. The measured concentration can be found in the sixth column. The table shows the accuracy of a claimed sandwich. Additionally, the table shows the ability of the claimed assay to accurately measure AMH at high and low concentrations.

FIG. 14 shows a Passing Bablok Regression Analysis between AMH concentrations measured at samples stored at −20° C. v. −70° C.: (ng/mL, N=10, Slope=1.06, Intercept=0.84) The slope is close to 1, showing almost no variability between AMH samples stored at −70° C. or −20° C.

FIG. 15 shows a Passing Bablok Regression Analysis between AMH concentrations measured at samples stored at −20° C. v. room temperature: (ng/mL, N=10, Slope=1.04, Intercept=0.89) The slope is close to 1, showing almost no variability between AMH samples stored at −20° C. or room temperature.

FIG. 16 shows a Passing Bablok Regression Analysis between AMH concentrations measured at samples stored at −20° C. v. 30° C.: (ng/mL, N=10, Slope=1.08, Intercept=0.95) The slope is close to 1, showing almost no variability between AMH samples stored at −20° C. or 30° C.

FIG. 17 shows the change in AMH concentrations of individual serum samples studied for stability at −20° C., −70° C., room temperature (RT), and 30° C. for 24 hours. Serum samples are labeled as S1-S3, S6 and diluted samples are labeled as S4 (1:2), S4 (1:4), S5 (1:2), S5 (1:4), S6 (1:2), S6 (1:4).

FIG. 18 shows the change in AMH concentration of individual serum samples studied for stability at −20° C., −70° C., room temperature (RT), and 30° C. for 72 hours. The serum samples are labeled as S1 and S2.

FIG. 19 is a western blot performed to determine where antibody #9 binds to AMH. Antibodies #9 cleanly and specifically detected the human AMH pro region in both human reduced (monomeric) and non-reduced (dimeric) forms.

FIG. 20 is a western blot performed to determine where antibody #10 binds to AMH. Antibodies #10 cleanly and specifically detected the human AMH pro region in both human reduced (monomeric) and non-reduced (dimeric) forms.

FIG. 21 is a western blot performed to determine where antibody #17 binds to AMH. Antibody #17 detects both monomeric and dimeric mature regions for both human and rat AMH.

FIG. 22 is a western blot performed to determine where antibody #17 binds to AMH. Antibody #17 detects both monomeric and dimeric mature regions for both human and rat AMH.

FIG. 23 shows the clinical application of the claimed AMH assays. It demonstrates ability of an SES assay to detect AMH in polycystic ovary syndrome (PCOS) positive (R1) and PCOS negative (R2) samples. It also demonstrates the advantage of measuring AMH fragments in a sample versus measuring full-length AMH in the sample. (See 36/33 R1 and R2 versus 36/33-Frag R1 and R2). Fragmenting the sample results in better differentiation between the PCOS negative and PCOS positive samples.

FIG. 24 shows a Passing Bablok Regression Analysis between AMH concentrations before and after fragmentation. (N=18, Slope=2.53, Intercept=0.69) in PCOS positive (R1) and PCOS negative (R2) samples using sandwich immunoassays with and without in situ fragmentation of the pro and mature AMH. After fragmentation, 2.5 times more AMH is detected in otherwise identical sample conditions using an identical sandwich immunoassay.

FIG. 25 shows examples of more clinical applications of the claimed AMH assays. In post-menopausal women, follicle levels are close to zero and the AMH concentration is undetectable. The claimed assays can be used to measure velocity of menopause by comparing the measured AMH level to the average level in a normal population at the same age. These values can be compared in order to predict when and how quickly a woman will develop menopause.

The table also demonstrates that in pediatric males, the AMH concentrations are very high, with values 10-20 times greater than adult males. Samples can be compared to this data to determine if a child has an intersex disorder.

FIGS. 26A-26C show the results of indirect ELISA assays to detect the binding of three different AMH mAbs to AMH peptides to identify the epitopes bound by the mAbs.

FIG. 27 shows a summary of AMH region-specific epitopes and the results of mAb binding to the epitopes.

FIG. 28 shows to localization of selected epitopes within the human AMH amino acid sequence.

FIG. 29 presents a table of AMH monoclonal antibodies (mAbs) and the amino acid sequences containing epitopes bound by the antibodies.

FIG. 30 shows several pairwise combinations of region-specific AMH mAbs suitable for sandwich ELISA assays to detect different forms of AMH.

FIG. 31 shows results of testing clinical samples using a next generation AMH assay.

FIG. 32 shows measurements of AMH in blood of normal human subjects using antibody pairs specific for midpro/midpro and midpro/mature regions. Plotted along the x-axis is the concentration of total AMH determined by sandwich ELISA using the antibody pair 24-37, each antibody binding to an epitope in the midpro region (amino acids 230-451). Plotted along the y-axis is the concentration of uncleaved AMH determined by sandwich ELISA using the antibody pair 24-32. Antibodies 24 and 32 bind to the midpro and the mature region respectively. The diagonal line corresponds to the expected result where all AMH in the samples is the uncleaved form of AMH.

FIG. 33 shows measurements of AMH in blood of human subjects with PCOS. The concentration of total AMH as determined by sandwich ELISA using the antibody pair 24-37 is plotted along the x-axis, each antibody binding to an epitope in the midpro region (amino acids 230-451). Plotted along the y-axis is the concentration of uncleaved AMH determined by sandwich ELISA using the antibody pair 24-32. Antibodies 24 and 32 bind to the midpro and the mature region, respectively. The diagonal line corresponds to the expected result where all AMH in the samples is the uncleaved form of AMH.

FIG. 34A is a linearity plot for an assay used to measure AMH in the blood of PCOS patients. Measurements were performed at two dilutions, 1:30 and 1:60, using the antibody pair 24-37. Both antibodies bind to epitopes in the midpro region. The slope (0.5157) of the linear fit shows near perfect linearity for a two-fold dilution.

FIG. 34B is a linearity plot for an assay used to measure AMH in the blood of PCOS patients. Measurements were performed at two dilutions, 1:30 and 1:120, using the antibody pair 24-37. Both antibodies bind to epitopes in the midpro region. The slope (0.237) of the linear fit shows near perfect linearity for a four-fold dilution.

FIG. 34C shows a linearity plot for an assay to measure AMH in the blood of PCOS patients. Measurements were performed at two dilutions, 1:30 and 1:60, using the antibody pair 24-32. Antibodies 24 and 32 bind to epitopes in the midpro and mature regions respectively. The slope (0.3764) of the linear fit is consistent with a two-fold dilution.

FIG. 35 shows a comparison of an AMH ELISA according to the present invention and a commercially available AMH ELISA (Beckman Coulter, Inc. (Brea Calif.) Gen II).

FIG. 36 is a graph showing the correlation between measurements of AMH concentration obtained from dried blood samples (y-axis) and those obtained from corresponding serum samples (x-axis).

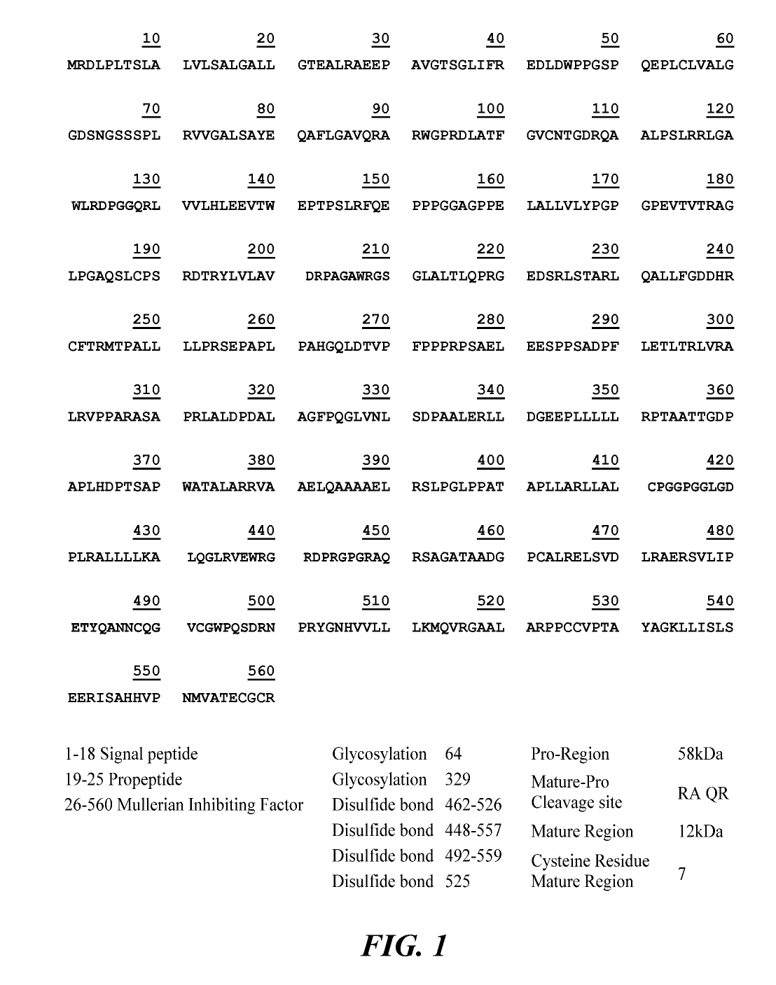

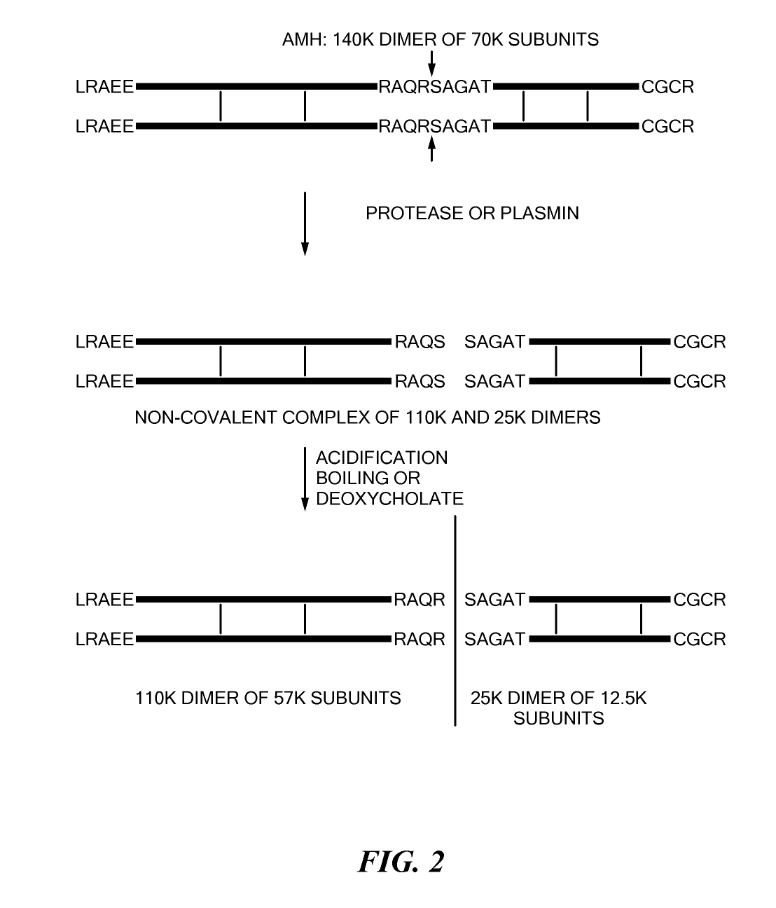

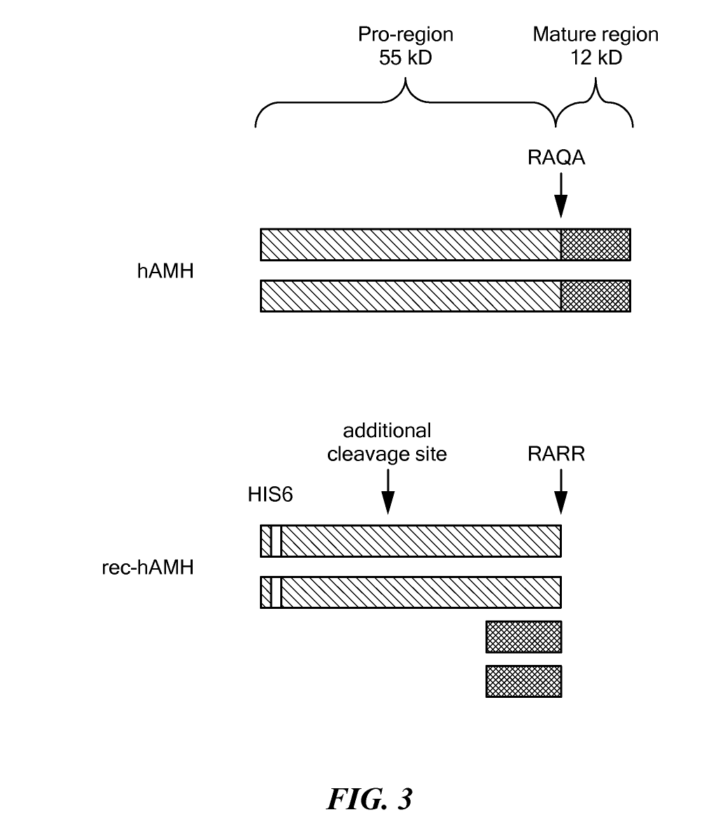

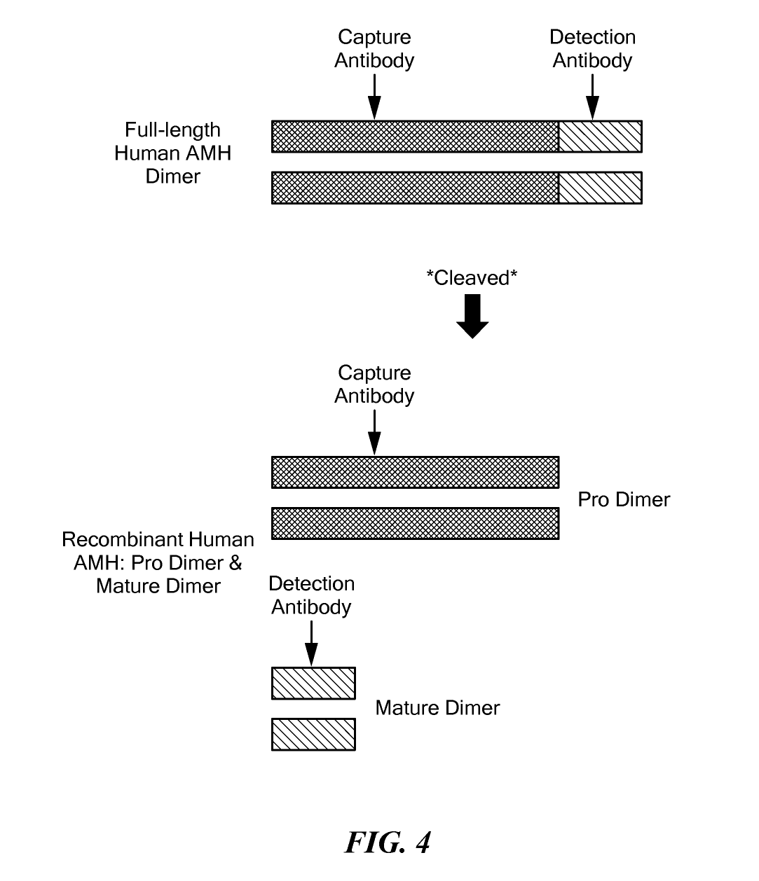

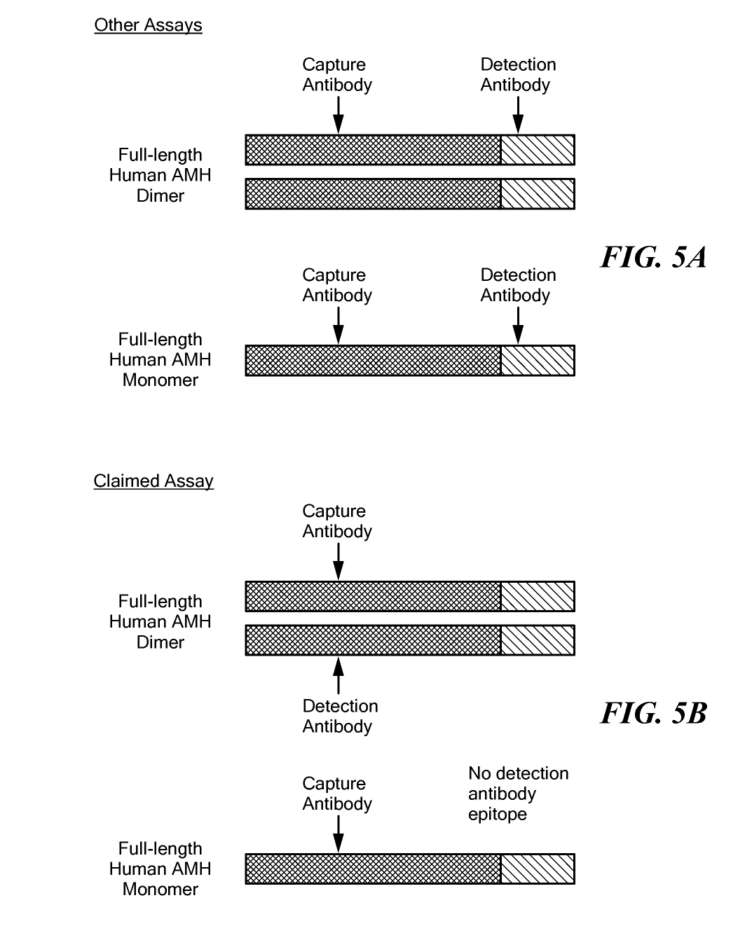

### DETAILED DESCRIPTION OF THE INVENTION

Monoclonal antibodies binding to defined regions of human AMH protein are described. Several monoclonal antibodies recognizing these peptide regions have been generated. These antibodies make it possible to detect and quantify various isoforms of AMH in a biological sample. These isoforms are: dimeric AMH; monomeric AMH; pro fragment of AMH; mature fragment of AMH; cleaved-reassociated AMH; and uncleaved pro-mature AMH. The mid-pro fragment also can be detected and quantified. Various assays to quantify the different isoforms of AMH are described.

Among the peptide regions described here there are a subset of AMH peptide regions containing epitopes, many of them linear epitopes, that remain surface accessible in each monomer of a dimeric AMH molecule and are therefore useful in a dimer-specific AMH assay. Although in general, subunits of a homodimeric and other oligomeric proteins are arranged symmetrically, there are many examples of protein dimers in which this symmetry is broken (Jerry Brown, Protein Science, 2006). In such cases an epitope accessible to an antibody specific to that epitope on one monomer may not be accessible to that antibody on the other monomer of the dimer. Without being limited by any particular theory or mechanism, an AMH dimer may be an asymmetric dimer, and therefore, only a subset of epitopes of an antibody on one monomer may be accessible to the antibody on the other monomer. Peptide regions of AMH are described here that contain epitopes that are accessible on both monomers of the AMH dimer. Such epitopes are useful for detecting and quantifying dimeric AMH using sandwich ELISA in which the same epitope is used for binding to both the capture and the detection antibodies. Described here also are monoclonal antibodies that bind to these epitopes and assays to detect AMH dimer using antibodies binding to these epitopes.

Additional AMH peptide regions are described that contain epitopes which are only accessible in monomeric AMH. Monoclonal antibodies that bind to these epitopes are also described herein. These antibodies are useful for detecting the monomeric forms of AMH.

Antibodies described herein may be paired in different ways to detect and quantify various isoforms of AMH in sandwich ELISA assays. For example, a sandwich ELSA using a capture antibody that binds an epitope in the pro region and a detection antibody that binds an epitope in the mature region, or vice versa, will allow detection of the uncleaved or the cleaved-reassociated form of AMH (dimeric or monomeric). On the other hand, a sandwich ELISA that uses an antibody pair that binds to different epitopes in the pro region, will measure all AMH isoforms that include the pro region (both dimeric and monomeric forms).

Antibodies described herein allow design of assays of quantification of AMH that yield substantially the same result, accounting for dilution, as the sample is diluted over a wide range of AMH concentrations, for example from about 20,000 pg/ml to about 1 pg/ml. Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would be a routine undertaking for those of ordinary skill in the art having the benefit of this disclosure. Other aspects, features, and advantages of the present invention will be apparent from the following description of the currently preferred embodiments of the invention. They are given for the purpose of disclosure, but are not meant to limit the scope of the invention.

AMH-Based Immunoassays

Embodiments of the invention as disclosed herein may be used to perform immunoassays that are often called “sandwich” immunoassays, wherein the analyte may be bound to or sandwiched between two antibodies that bind to opposite sides of the analyte. Examples of such immunoassays include, but are not limited to, enzyme immunoassays or enzyme-linked immunosorbent assays (EIA or ELISA), chemiluminescent assays, immunoradiometric assays (IRMA), fluorescent immunoassays, lateral flow assays, diffusion immunoassays, dried spot assays, immunoprecipitation assays, and assays utilizing magnetic separation techniques. The immunoassays of the invention can be performed, for example, in microtiter plate format as well as in rapid test format with one or more “capture” antibodies bound to the plate or a porous membrane to capture one or more isoforms of AMH and one or more labeled “detection” antibodies added for detection and/or quantification of the captured AMH.

Another embodiment of the present invention provides a method for performing a single epitope sandwich (SES)-based AMH assay utilizing antibodies that bind to a single epitope of anti-Müllerian hormone protein that is resistant to proteolysis. In such assays, the amount of the anti-Müllerian hormone measured in the sample is not affected by proteolysis of the AMH protein in the sample. The “capture” and “detection” antibody epitopes are the same—they will never be on opposite ends of a cleavage site. Thus, an SES immunoassay can accurately measure all forms of AMH protein in samples, regardless of the particular type of assay conditions. In preferred embodiments of the invention, the antibodies used in the SES immunoassay are directed either to the pro or the mature dimeric form of AMH.

Another embodiment of the present invention provides a method for measuring isoforms of AMH by first separating the full-length AMH into mature and pro fragments in situ, and measuring the amount of fragments in the sample, versus the amount of full-length AMH.

Another embodiment provides a method of measuring AMH in a biological sample in order to diagnose a clinical condition. Such clinical conditions may include, but are not limited to: ovarian reserve in an IVF setting, ovarian function, oocyte quality, premature ovarian failure, premature ovarian failure, ovarian insufficiency, ovarian granulosa cell tumors, breast cancer, ovarian function for childhood cancer survivors, polycystic ovary syndrome, menopause, and intersex disorders.

Another embodiment provides a method of measuring AMH in a biological sample; comparing the measurement of Anti-Müllerian Hormone to levels of other biomarkers, including but not limited to, Inhibin B and FSH; and using the combination of biomarkers to assess a clinical condition.

Another embodiment provides a method of measuring AMH in a biological sample with an algorithm in order to assess a clinical condition.

Hybridoma Deposit

AMH mouse spleen hybridoma denoted herein as “33/1A” has been deposited under conditions that assure that access to the cultures will be available during the pendency of this patent application to one determined by the Commissioner of Patents and Trademarks to be entitled thereto under 37 C.F.R. §1.14 and 35 U.S.C. §122. The deposit is available as required by foreign patent laws in countries wherein counterparts of the subject application, or its progeny, are filed. However, it should be understood that the availability of a deposit does not constitute a license to practice the subject invention in derogation of patent rights granted by governmental action. The subject culture deposit will be stored and made available to the public in accord with the provisions of the Budapest Treaty for the Deposit of Microorganisms, i.e., it will be stored with all the care necessary to keep it viable and uncontaminated for a period of at least five years after the most recent request for the finishing of a sample of the deposit, and in any case, for a period of at least 30 (thirty) years after the date of deposit or for the enforceable life of any patent which may issue disclosing the deposited culture. The depositor acknowledges the duty to replace the deposit should the depository be unable to furnish a sample when requested, due to the condition of the deposit. All restrictions on the availability to the public of the subject culture deposit will be irrevocably removed upon the granting of a patent disclosing it. A deposit of AMH mouse spleen hybridoma 33/1A was entered into the permanent collection of the Patent Depository of the American Type Culture Laboratory, located at 10801 University Blvd., Manassas, Va., 20110-2209, USA, on Jul. 24, 2012 on behalf of Ansh Labs, LLC, under the terms of the Budapest Treaty, whereupon it was assigned accession number ATCC PTA-13088 by the repository. The deposit was tested by the repository on Aug. 6, 2012, and on that date, the strains were certified viable.

Antibody Compositions

In the practice of the invention, IgG and/or IgM antibodies are particularly contemplated to be useful, not only because they are the most common antibodies physiologically, but also because they are most readily-generated in a laboratory facility. Although the “light chains” of mammalian antibodies fall into one of two clearly-distinct types: kappa (κ) or lambda (λ), based on the amino acid sequence of their constant domains, there is essentially no preference for the use of one form over the other in the preparation of antibodies in accordance with various aspects of the present invention.

The use of monoclonal antibodies (MAbs), or derivatives thereof, represents a preferred aspect of the present invention. MAbs are recognized to have certain advantages (e.g., reproducibility and facile, large-scale production), making them particularly well-suited for therapeutic and diagnostic applications. While the invention provides monoclonal antibodies of any suitable origin (including, for example, murine, human, non-human primate, rat, hamster, rabbit, and even frog or chicken origin), murine, human, or alternatively, “humanized” antibodies are of particular use in the various immunological, diagnostic, and/or therapeutic methods of the present invention.

As is readily appreciated by those of ordinary skill in the art, the immunological binding reagents encompassed by the term “antibody” extend to all antibodies from all species, and antigen binding fragments thereof, including dimeric, trimeric and multimeric antibodies; bispecific antibodies; chimeric antibodies; human and humanized antibodies; recombinant, engineered and camelized (i.e., camelized) antibodies, and fragments thereof. The term “antibody” is thus used to refer to any antibody-like molecule that has an antigen binding region, including, for example molecules such as antibody fragments (e.g., Fab′, Fab, F(ab′)₂, single domain antibodies (DABs), Fv, scFv (single chain Fv), linear antibodies, diabodies, camelized antibodies and the like). The techniques for preparing and using various antibody-based constructs and fragments are well-known to those of ordinary skill in the art.

Preferred antibodies of the present invention preferably include those that bind to an AMH peptide or polypeptide, and that comprise at least one CDR of an antibody provided herein, preferably the anti-AMH antibody referred to herein as “antibody #9” and obtainable from the AMH mouse spleen hybridoma 33/1A. For example, the present invention provides antibodies that preferably specifically bind to active, dimeric, Anti-Müllerian Hormone polypeptides, and that comprise at least one CDR from the monoclonal antibody produced by the hybridoma deposited with the American Type Culture Collection as PTA-13088; or at least one CDR that has the amino acid sequence of SEQ ID NO:2 or SEQ ID NO:7, or an amino acid sequence that is encoded by the polynucleotide sequence of SEQ ID NO:1 or SEQ ID NO:2, or a variant, a derivative, or a mutagenized form of the amino acid sequence in accordance with SEQ ID NO:2 or SEQ ID NO:7, wherein such a variant, derivative, or mutagenized form maintains binding, and preferably, substantially maintains binding (and preferably, specific binding) to at least a first AMH, or dimeric AMH peptide or polypeptide.

Additionally preferred antibodies of the present invention preferably include those that bind to an AMH peptide or polypeptide, and that comprise at least one CDR of an antibody provided herein, preferably the anti-AMH antibody referred to herein as “antibody #10”. For example, the present invention provides antibodies that preferably specifically bind to active, dimeric, Anti-Müllerian Hormone polypeptides, and that comprise at least one CDR from the monoclonal antibody #10; or at least one CDR that has an amino acid sequence in accordance with or any one of SEQ ID NO:12-17, or that has an amino acid sequence that is encoded by the polynucleotide sequence of SEQ ID NO: 11 or SEQ ID NO:16, or a variant, a derivative, or a mutagenized form of the amino acid sequence in accordance with any one of the amino acid sequences of SEQ ID NO:12 or SEQ ID NO:17, or an amino acid sequence that is encoded by the polynucleotide sequence of any one of SEQ ID NO: 11 or SEQ ID NO:16, wherein such a variant, derivative, or mutagenized form maintains binding, and preferably, substantially maintains binding (and preferably, specific binding) to at least a first Müllerian hormone, or dimeric Müllerian hormone polypeptide.

In certain embodiments, so-called “second-generation” antibodies are provided that have enhanced or superior properties in comparison to an original anti-AMH antibody, such as the antibody produced from the AMH mouse spleen hybridoma 33/1A and referred to herein as antibody #9 (and deposited with the ATCC under accession number PTA-13088). Particularly included among such second-generation antibodies include those antibodies that comprise at least one complementarity determining region (CDR) that includes one or more variants, derivatives, or mutagenized forms of one or more of the amino acid sequences as set forth in any one of SEQ ID NO:2-17, or an amino acid sequence that is encoded by the polynucleotide sequence of any one of SEQ ID NO:1, SEQ ID NO:6, SEQ ID NO: 11 or SEQ ID NO:16, provided that such a variant, derivative, or mutagenized form substantially maintains at least substantial binding affinity to one or more Müllerian hormone-specific peptides or polypeptides.

Anti-Müllerian hormone antibodies according to the present invention thus include those antibodies that comprise at least a first variable region that includes an amino acid sequence region of at least about 75%, more preferably, at least about 80%, more preferably, at least about 85%, more preferably, at least about 90% and most preferably, at least about 95% or so amino acid sequence identity to an amino acid sequence in accordance with or any one of SEQ ID NO:2-17, or an amino acid sequence that is encoded by the polynucleotide sequence of any one of SEQ ID NO:1, SEQ ID NO:6, SEQ ID NO: 11 or SEQ ID NO:16; wherein the anti-Müllerian hormone antibody at least substantially maintains the biological properties, and or the binding characteristics and/or binding specificity of at least one of the anti-AMH antibodies as set forth in the present invention, and particularly exemplified by the AMH antibodies #9 and #10 described herein, including, for example, anti-AMH antibody #9, produced by mouse spleen hybridoma line 33/1A (deposited as PTA-13088 with the ATCC).

Identity or homology with respect to these and other anti-Müllerian hormone antibody sequences of the present invention is defined herein as the percentage of amino acid residues in a candidate sequence that are identical to an amino acid sequence in accordance with or any one of SEQ ID NO:2-17, or is identical to an amino acid sequence that is encoded by the polynucleotide sequence of any one of SEQ ID NO:1, SEQ ID NO:6, SEQ ID NO: 11 or SEQ ID NO:16, or to the sequence of another anti-Müllerian hormone antibody of the invention, after aligning the sequences and introducing gaps, if necessary, to achieve the maximum percent sequence identity. Particularly important, is the maintenance of substantially the same, or more preferably, even improved, biological properties of the anti-Müllerian hormone antibody used for the sequence comparison. Such comparisons may be readily conducted using conventional immunological assays, e.g., one or more of the antibody-based assays described in detail herein.

In further embodiments, the antibodies employed will either be “humanized,” part-human or fully-human antibodies. “Humanized” antibodies are generally chimeric monoclonal antibodies derived from a non-human species (e.g., a mouse or a rat), bearing one or more human constant and/or variable region domains (i.e., “part-human” chimeric antibodies, for example). Various humanized monoclonal antibodies suitable for use in the present invention include, without limitation, chimeric antibodies wherein at least a first antigen binding region, or complementarity determining region (CDR), of a non-human monoclonal antibody is operatively attached to, or “grafted” onto, a human antibody constant region or “framework”.

“Humanized” monoclonal antibodies suitable for use in the practice of the invention may include monoclonal antibodies from non-human species wherein one or more selected amino acids have been conservatively substituted for one or more amino acids that are more commonly represented in native, human antibodies. Such “humanization” of antibodies is a widely-used technique in the immunological arts, and can be readily achieved, for example, through the use of recombinant molecular biology techniques, including, for example, site-specific mutagenesis, and the like, the protocols and methods for which are well-known to those of ordinary skill in the art.

Entirely human (as opposed to “humanized”) antibodies may also be prepared and used in accordance with various aspects of the present invention. Such human antibodies may be obtained from healthy subjects simply by obtaining a population of mixed peripheral blood lymphocytes from a human subject, including antigen-presenting and antibody-producing cells, and stimulating the cell population in vitro by admixing with an immunogenically effective amount of a sample containing at least a first anti-Müllerian hormone. The human anti-Müllerian hormone antibody-producing cells, once obtained, may then be employed in one or more conventional hybridoma and/or recombinant antibody production methods using methods that are well-known to those of ordinary skill in the immunological arts.

Further techniques for human monoclonal antibody production include immunizing a transgenic animal, preferably a transgenic mouse, which comprises a human antibody library with an immunogenically effective amount of an anti-Müllerian hormone peptide, polypeptide, antibody, or fragment thereof-containing sample. This also generates human anti-Müllerian hormone antibody-producing cells for further manipulation in hybridoma and/or recombinant antibody production, with the advantage that spleen cells, rather than peripheral blood cells, can be readily obtained from the transgenic animal or mouse.

Antibodies in accordance with the invention may be readily prepared by selecting an antibody that substantially cross-reacts or competes with the AMH monoclonal antibody 33/1A (obtainable from the mouse spleen hybridoma deposited with the ATCC on Jul. 24, 2012 as accession number PTA-13088). Suitable preparative processes and methods generally include the steps of (a) preparing candidate antibody-producing cells; and (b) selecting from the candidate antibody-producing cells an antibody that substantially cross-reacts or competes with the AMH monoclonal antibody produced by the hybridoma line 33/1A (ATCC PTA-13088).

Suitable antibody-producing cells may also be obtained, and antibodies subsequently isolated and/or purified, by stimulating peripheral blood lymphocytes with one or more dimeric Müllerian hormone compositions in vitro. Other methods comprise administering to an animal an immunizing composition comprising at least a first immunogenic dimeric Müllerian hormone polypeptide composition, and selecting from the immunized animal an antibody that substantially cross-reacts or competes with the anti-AMH monoclonal antibody produced by the murine hybridoma line 33/1A (ATCC PTA-13088). These methods generally comprise: (a) immunizing an animal by administering to the animal at least one dose, and optionally more than one dose, of a composition comprising an immunogenically-effective amount of an immunogenic Müllerian hormone dimeric polypeptide composition; and (b) obtaining a suitable antibody-producing cell from the immunized animal, such as an antibody-producing cell that produces an antibody that substantially cross-reacts or competes with the AMH monoclonal antibody 33/1A as produced by the hybridoma line deposited with the ATCC as PTA-13088.

In the practice of the invention, anti-AMH antibodies in accordance with the present invention may be optionally labeled for use in a variety of diagnostic, immunologic, and/or localization assays. Suitable detectable labels include, without limitation, chromogenic labels, fluorogenic labels, or enzyme-substrate labels, including rhodamine, streptavidin, fluorescein and its derivative, or one or more X-ray detectable compound, such as bismuth (III), gold (III), lanthanum (III) or lead (II); a radioactive ion, such as copper⁶⁷, gallium⁶⁷, gallium⁶⁸, indium¹¹¹, indium¹¹³, iodine¹²³, iodine¹²⁵, iodine¹³¹, mercury¹⁹⁷, mercury²⁰³, rhenium¹⁸⁶, rhenium¹⁸⁸, rubidium⁹⁷, rubidium¹⁰³, technetium^{99m} or yttrium⁹⁰; or a nuclear magnetic spin-resonance isotope, such as cobalt (II), copper (II), chromium (III), dysprosium (III), erbium (III), gadolinium (III), holmium (III), iron (II), iron (III), manganese (II), neodymium (III), nickel (II), samarium (III), terbium (III), vanadium (II) or ytterbium (III), or any combination thereof.

For antibody- and peptide-based conjugates, the term “conjugate” is generally used to define the operative association of the antibody or peptide with another effective agent and is not intended to refer solely to any type of operative association, and is particularly not limited to chemical “conjugation.” Recombinant fusion proteins are particularly contemplated, and so long as the antibody or peptide is able to bind to the target dimeric Müllerian hormone polypeptide composition, and the attached agent functions sufficiently as intended (particularly when delivered to a selected target site, for example), any conventional mode of attachment known to those of ordinary skill in the art may be employed to create anti-AMH-based immunoconjugates.

The invention further provides compositions comprising at least a first purified anti-AMH antibody, antigen-binding fragment, or immunoconjugate thereof, and optionally, one that binds essentially to the same epitope as the AMH monoclonal antibody 33/1A as produced by the hybridoma line deposited with the ATCC as PTA-13088. Such compositions preferably comprise a biologically-effective, diagnostically-effective, and/or therapeutically-effective amount of any such agent, such as an amount effective to bind one or more target anti-Müllerian hormone-derived antigens, epitopes, or such like.

Aspects of the invention further include compositions, pharmaceutical compositions, combinations, mixtures, medicaments and/or medicinal cocktails of agents, comprising at least a first purified anti-AMH antibody, a first anti-AMH-derived antigen-binding fragment, or a first purified anti-AMH immunoconjugate, including, for example, those antibodies and antigen binding fragments that bind essentially to the same epitope as the monoclonal antibody 33/1A, in combination with a biologically-, diagnostically- or therapeutically-effective amount of at least a second component, compound, composition, or biological agent.

Further embodiments of the invention concern kits comprising, in at least a first composition or suitable container, at least a first purified anti-AMH antibody, or an anti-AMH antigen-binding fragment, or an anti-AMH immunoconjugate, or any combination thereof, in combination with a biologically-, diagnostically- and/or therapeutically-effective amount of at least a second biological agent, component or system. As used herein, the “second biological agent, component or system” need not be limited to conventional therapeutic or diagnostic agents. For example, second biological agents, components or systems may include one or more components for modification of the anti-AMH antibody, and/or one or more components for attaching one or more additional agents, such as a detectable label, ligand, or targeting moiety, to the anti-Müllerian hormone antibody.

The second diagnostic agent, component or system may also include one or more diagnostic agents, components or systems directly or indirectly detectable by one or more in vitro, in vivo, or in situ diagnostic test or procedure. “Directly detectable reporter agents” include, without limitation, radiolabels, reporter agents detectable by immunofluorescence, luciferase, and the like. “Indirectly detectable reporter agents” function in conjunction with further exogenous agent(s), such as detectable enzymes that yield a colored product on contact with a chromogenic substrate. These include, without limitation, “secondary antibodies” that are attached to one or more directly- or indirectly-detectable agent(s), such as, without limitation, a radiolabel or enzyme, and “secondary and tertiary antibody detection systems,” in which the tertiary antibody is attached to the detectable agent.

Preferred diagnostic kits in accordance with the present invention including, without limitation, those comprising one or more diagnostic agents, components or systems that may be detected, or quantified by one or more in vivo or in situ diagnostic and/or imaging methodologies. The kits of the present invention may therefore comprise combined biologically-diagnostically- and/or therapeutically-effective amounts of at least the two specified agents within a single container, or alternatively, within distinct containers. The kits may also comprise instructions for using the biological agents and anti-AMH antibodies included therein. Imaging components may also be included in combination, but separately with the therapeutic kits.

Preparation of Anti-AMH Antibody Compositions

The anti-AMH antibody compositions of the present invention find particular utility in a variety of diagnostic and immunodetection-based assays. The production of antibodies specific for the AMH dimeric polypeptides described herein and/or polypeptides encoded by the polynucleotides of the invention, including e.g., those described herein (as well as conservative variants thereof) represent an important aspect of the present invention. AMH antibodies specific for dimeric forms of the molecule are useful, e.g., in both diagnostic and therapeutic/prophylactic purposes, e.g., related to the activity, distribution, and expression of target AMH polypeptides, and in the differentiation and/or quantitation of monomeric vs. dimeric forms of the molecule in one or more selected cells, tissues, or fluids of an individual, including, for example, human subjects.

Antibodies specific for the dimeric AMH polypeptides of the invention can be generated by methods well known to those of ordinary skill in the immunological arts. Such antibodies can include, but are not limited to, polyclonal, monoclonal, chimeric, humanized, single chain, Fab fragments, and fragments produced by an Fab expression library. Numerous methods for producing polyclonal and monoclonal antibodies are known to those of ordinary skill in the art, and can be adapted to produce antibodies specific for the polypeptides of the invention, and/or encoded by the polynucleotide sequences of the invention (see, e.g., Coligan Current Protocols in Immunology Wiley/Greene, NY, Paul (ed.) (1991); (1998) Fundamental Immunology Fourth Edition, Lippincott-Raven, Lippincott Williams & Wilkins; Harlow and Lane (1989) Antibodies: A Laboratory Manual, Cold Spring Harbor Press, NY, USA; Stites et al. (Eds.) Basic and Clinical Immunology (4th ed.) Lange Medical Publications, Los Altos, Calif., USA and references cited therein; Goding, Monoclonal Antibodies: Principles and Practice (2d ed.) Academic Press, New York, N.Y., USA; 1986; and Kohler and Milstein (1975). For certain applications, preparation of “humanized” antibodies may be desirable. Methods for the preparation of chimeric (humanized) antibodies are known to those of ordinary skill in the art, and are exemplified e.g., in U.S. Pat. Nos. 4,634,664; 4,634,666; and 5,482,856, each of which is specifically incorporated herein in its entirety by express reference thereto.

In particular embodiments, antibodies, either monoclonal or polyclonal, that specifically bind to dimeric forms of AMH polypeptides, but preferably do not specifically bind to monomeric or other forms of the AMH molecules represent important aspects of the invention. Techniques for preparing and characterizing antibodies are well known in the art, particularly with the guidance provided herein. The methods for generating monoclonal antibodies (MAbs) generally begin along the same lines as those for preparing polyclonal antibodies. Briefly, a polyclonal antibody is prepared by immunizing an animal with an immunogenic composition in accordance with the present invention and collecting antisera from that immunized animal. A wide range of animal species can be used for the production of antisera. Typically the animal used for production of anti-antisera is a rabbit, a mouse, a rat, a hamster, a guinea pig or a goat. Because of the relatively large blood volume of rabbits, a rabbit is a preferred choice for production of polyclonal antibodies.

As is well known to those of ordinary skill in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Methods for conjugating a polypeptide of the invention to a carrier protein are well known to those of ordinary skill in the art, and include, e.g., glutaraldehyde, m-maleimidobencoyl-N-hydroxysuccinimide ester, carbodiimide and bis-biazotized benzidine, or any combination thereof.

The amount of immunogenic composition used in the production of polyclonal antibodies varies upon the nature of the immunogen as well as the animal used for immunization. Any of the routes disclosed herein can be used to administer the AMH epitopic sequence, AMH-peptide, or AMH immunogenic composition to produce one or more anti-AMH antibodies having the desirable properties described herein (including, for example, the specificity for binding to dimeric, but not monomeric or other forms of the AMH protein). The production of AMH-specific polyclonal antibodies may be monitored by sampling blood of the immunized animal at various points following immunization. One or more subsequent, or “booster”, injections may also be administered to the production animal as necessary to achieve the desired antibody production. The process of boosting and titering of antibodies is known to those of ordinary skill in the art, and may be repeated as necessary until a suitable titer of the anti-AMH antibodies is achieved. When a desired level of immunogenicity is obtained, the immunized animal can be bled and the serum isolated and stored, and/or the animal can be used to generate MAbs.

MAbs may be readily prepared through use of well-known techniques, such as those exemplified in U.S. Pat. No. 4,196,265, specifically incorporated herein in its entirety by express reference thereto. Typically, this technique involves immunizing a suitable animal with a selected immunogen composition, e.g., a purified or partially-purified AMH protein, polypeptide or peptide. The AMH immunizing composition is preferably administered in a manner effective to stimulate AMH antibody-producing cells. Rodents such as mice and rats are preferred animals; however, the use of rabbit, sheep, frog, and other host organisms and/or cell lines is also possible. The use of rats may provide certain advantages, but mice are often the most preferred sources and most routinely used, as they often produce a higher percentage of stable fusions.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The AMH antibody-producing B lymphocytes from the immunized animal can then be fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media that support the growth of only the desired fused cells (hybridomas).

Any one of a number of myeloma cells may be used, as are known to those of ordinary skill in the art. For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions. One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line, which is widely employed in the art, and known to those of ordinary skill in the antibody field. Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described in the art, and those using polyethylene glycol (PEG), such as 37% (vol./vol.) PEG.

Fusion procedures usually produce viable hybrids at low frequencies, about 1×10⁻⁶ to 1×10⁻⁸. This does not pose a problem, however, as the viable, fused hybrids are typically differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine, and combinations thereof. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The selected hybridomas are then serially diluted and cloned into individual antibody-producing cell lines, which clones can then be propagated indefinitely to provide MAbs. The cell lines may be exploited for MAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. MAbs produced by either means may be further purified, if desired, using filtration, centrifugation and various chromatographic methods such as HPLC or affinity chromatography.

In exemplary embodiments, murine spleen cell-based hybridomas have been developed and used to produce the anti-AMH antibodies of the present invention. One such hybridoma, 33/A1 is described in detail elsewhere herein, and was deposited into the permanent patent repository of the American Type Culture Collection. Details on the particular hybridoma, and the resulting antibodies produced therefrom are described in the Examples which follow.

AMH-Specific Polynucleotide Compositions

Any polynucleotide that encodes one or more of the AMH epitopic peptides or polypeptides as described herein, or that encodes one or more of the CDRs of an anti-AMH antibody or antibody binding fragment thereof, or a polynucleotide that is substantially complementary to such a polynucleotide is also encompassed by the present invention. Such polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials. The polynucleotides of the invention may encode one or more AMH-derived epitopic peptides or antigens, or may encode an entire AMH immunogenic peptide or polypeptide that comprises a plurality of individual epitopes and/or smaller peptide antigens, or may encode a variant of one or more such peptides or polypeptides as described herein, or may encode one or more AMH-specific antibodies of the present invention, or alternatively, encode one or more of the CDRs comprising one of more of the disclosed AMH-specific antibody compositions. Polynucleotide variants may contain one or more substitutions, additions, deletions and/or insertions such that the immunogenicity of the encoded AMH peptide or the immunospecificity of the encoded anti-AMH antibody is not diminished, relative to the native protein. The effect on the immunogenicity of the encoded AMH peptide, or the effect on the immunospecificity of the encoded anti-AMH antibody may generally be assessed as described herein. Preferred peptide variants contain amino acid substitutions, deletions, insertions and/or additions at no more than about 20%, more preferably at no more than about 15%, and more preferably still, at no more than about 10% or 5% or less of the amino acid positions relative to the corresponding native unmodified amino acid sequence.

Likewise, polynucleotides encoding such peptide variants should preferably contain nucleotide substitutions, deletions, insertions and/or additions at no more than about 20%, more preferably at no more than about 15%, and more preferably still, at no more than about 10% or 5% or less of the nucleotide positions relative to the corresponding polynucleotide sequence that encodes the native unmodified amino acid sequence. Certain polynucleotide variants, of course, may be substantially homologous to, or substantially identical to the corresponding region of the nucleotide sequence encoding an unmodified peptide. Such polynucleotide variants are capable of hybridizing to a naturally occurring DNA sequence encoding one or more antigenic peptides as disclosed herein (or a complementary sequence) under moderately stringent, to highly stringent, to very highly stringent conditions.

Suitable moderately stringent conditions include, e.g., pre-washing in a solution containing about 5×SSC, 0.5% SDS, 1.0 mM EDTA (pH 8.0); hybridizing at a temperature of from about 50° C. to about 60° C. in 5×SSC overnight; followed by washing twice at about 60 to 65° C. for 20 min. with each of 2×, 0.5× and 0.2×SSC containing 0.1% SDS). Suitable highly stringent conditions include, e.g., pre-washing in a solution containing about 5×SSC, 0.5% SDS, 1.0 mM EDTA (pH 8.0); hybridizing at a temperature of from about 60° C. to about 70° C. in 5×SSC overnight; followed by washing twice at about 65 to 70° C. for 20 min. with each of 2×, 0.5× and 0.2×SSC containing 0.1% SDS). Representative examples of very highly stringent hybridization conditions may include, for example, pre-washing in a solution containing about 5×SSC, 0.5% SDS, 1.0 mM EDTA (pH 8.0); hybridizing at a temperature of from about 70° C. to about 75° C. in 5×SSC overnight; followed by washing twice at about 70° C. to about 75° C. for 20 min with each of 2×, 0.5× and 0.2×SSC containing 0.1% SDS). Such hybridizing DNA sequences are also within the scope of this invention.

It will be appreciated by those of ordinary skill in the art that, as a result of the degeneracy of the genetic code, there are many nucleotide sequences that encode a given primary amino acid sequence. Some of these polynucleotides bear minimal homology to the nucleotide sequence of any native gene. Nonetheless, polynucleotides that vary due to differences in codon usage are specifically contemplated by the present invention.

Immunogenic peptide-encoding polynucleotides, as well as antibody- or antigen binding fragment-encoding polynucleotides in accordance with the present invention may be synthesized by any method known in the art, including chemical synthesis (e.g., solid phase phosphoramidite chemical synthesis). Modifications in a polynucleotide sequence may also be introduced using standard mutagenesis techniques, such as oligonucleotide-directed site-specific mutagenesis. Alternatively, RNA molecules may be generated by in vitro or in vivo transcription of DNA sequences encoding an immunogenic composition as disclosed herein, provided that the DNA is incorporated into a vector with a suitable RNA polymerase promoter (such as T7 or SP6). Certain portions may be used to prepare an encoded peptide, as described herein. In addition, or alternatively, a portion may be administered to a patient such that the encoded peptide is generated in vivo (e.g., by transfecting antigen-presenting cells such as dendritic cells with a cDNA construct encoding one or more AMH-specific or AMH-antibody-derived immunogenic peptides, and administering the transfected cells to the patient).

Polynucleotides that encode an AMH polypeptide or an AMH-specific antibody may generally be used for production of such peptides, either in vitro or in vivo. Nucleotide sequences as described herein may be joined to a variety of other nucleotide sequences using established recombinant DNA techniques. For example, a polynucleotide may be cloned into any of a variety of cloning vectors, including one or more of plasmids, phagemids, lambda phage derivatives and cosmids. Vectors of particular interest include expression vectors, replication vectors, probe generation vectors, and sequencing vectors. In general, a vector will contain an origin of replication functional in at least one organism, convenient restriction endonuclease sites and one or more selectable markers. Other elements will depend upon the desired use, and will be apparent to those of ordinary skill in the art. Within certain embodiments, polynucleotides may be formulated so as to permit entry into a cell of a mammal, and expression therein. Those of ordinary skill in the art will appreciate that there are many ways to achieve expression of a selected AMH polynucleotide in a target cell, and any suitable method known to such artisans may be routinely employed in the practice of the invention. For example, an AMH-specific or AMH-antibody-specific polynucleotide may be incorporated into a viral vector such as, but not limited to, influenzavirus adenovirus, baculovirus, parvovirus, herpes virus, adeno-associated virus, retrovirus, flavivirus, vaccinia or poxvirus (e.g., avian poxvirus). Techniques for incorporating DNA into such vectors are well known to those of ordinary skill in the art. A viral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those of ordinary skill in the art.

Pharmaceutical Formulation of AMH Detection Reagents & Compositions

In certain embodiments, the present invention concerns formulation of one or more diagnostic, therapeutic, or immunodetection agents in one or more pharmaceutically-acceptable compositions for administration to a cell or an animal, either alone, or in combination with one or more other modalities. The formulation of pharmaceutically-acceptable excipients and carrier solutions is well known to those of ordinary skill in the art, as is the development of suitable regimens for using the particular compositions described herein in a variety of immunodetection-based methodologies. Sterile compositions may be prepared by incorporating the disclosed AMH compositions in the required amount in the appropriate solvent with several of the other ingredients enumerated above, as required, followed by conventional methods such as filter sterilization and the like.

The compositions disclosed herein are not in any way limited to use only in humans, or even to primates, or mammals. In preferred embodiments, however, the compositions of the present invention may be formulated for use in a variety of immunological methods applicable to mammalian cells, including in particular, human cells, for one or more diagnostic and/or immunodetection methodologies. The disclosed anti-AMH antibody compositions may also be formulated for use in a variety of non-human assay systems, including, for example, veterinary applications, including, for example, the analysis of biological samples from selected livestock, exotic or domesticated animals, companion animals (including pets and such like), non-human primates, as well as zoological or otherwise captive specimens, and such like.

### AMH-Based Kits and Commercial Formulations

Kits including one or more of the disclosed immunological compositions or pharmaceutical formulations including such; and instructions for using the kit in a diagnostic, therapeutic, and/or other immunological-based embodiment(s) also represent preferred aspects of the present disclosure. Such kits may include one or more of the disclosed antibody compositions, either alone, or in combination with one or more additional diagnostic compounds, pharmaceuticals, immunodetection reagents, and such like. The kits according to the invention may be packaged for commercial distribution, and may further optionally include one or more delivery devices for the composition(s) to an animal (e.g., syringes, injectables, or such like). Such kits may be diagnostic kits for detecting one or more AMH compositions in a sample, and may include instructions for using the kit in a diagnostic regimens, immunodetection protocols, and such like.

The container(s) for such kits may typically include at least one vial, test tube, flask, bottle, syringe or other container, into which the anti-AMH composition(s) disclosed herein may be placed, and, preferably, suitably aliquoted for convenient use and/or commercial sale. Where a second immunodetection composition is also desired, the kit may also contain the second immunodetection composition in a second distinct container, or a single container with a breakable or non-breakable barrier to isolate the two components. Alternatively, a plurality of distinct immunodetection composition(s) and/or distinct diagnostic compound(s) may be prepared in a single formulation, and may be packaged in a single container, vial, flask, syringe, catheter, cannula, bottle, test tube, ampoule, ELISA, microtiter plate, multi-well assay system, or other suitable container(s). The kit may also include a larger container, such as a case, that includes the containers noted above, along with other equipment, instructions, additional reagents, and the like.

Another important aspect of the present invention concerns methods for using the disclosed immunodetection reagents and anti-AMH antibody compositions (as well as formulations including them) in the preparation of medicaments for diagnosis, prevention, treatment, or amelioration of a disease, or the one or more symptoms thereof, in an animal, such as a vertebrate mammal Use of the disclosed immunodetection reagents and antibody compositions is also contemplated in various laboratory tests, diagnostic regimens, and/or immunodetection-based assay systems.

Sequence Comparison, Identity, and Homology

The terms “identical” or percent “identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of ordinary skill) or by visual inspection.

The phrase “substantially identical,” in the context of two nucleic acids or polypeptides (e.g., antigenic polypeptide sequences, or polynucleotide sequences that encode them) refers to two or more sequences or subsequences that have at least about 90%, preferably 91%, most preferably 92%, 93%, 94%, 95%, 96%, 97%, 98%, 98.5%, 99%, 99.1%, 99.2%, 99.3%, 99.4%, 99.5%, 99.6%, 99.7%, 99.8%, 99.9% or more nucleotide or amino acid residue identity, when compared and aligned for maximum correspondence, as measured using a sequence comparison algorithm or by visual inspection. Such “substantially identical” sequences are typically considered “homologous,” without reference to actual ancestry. With respect to peptides and polypeptides, preferably, “substantial identity” exists over a region of the amino acid sequences that is at least about 10, at least about 20, at least about 30, at least about 40, at least about 50, amino acid residues in length, or more, even up to and including the full length (or nearly the full length) of the two peptide or polypeptide sequences being compared. With respect to polynucleotides encoding such antigenic peptides and polypeptides, preferably, “substantial identity” exists over a region of the nucleic acid sequences that is at least about 30, at least about 60, at least about 90, at least about 120, at least about 150, nucleic acids in length, or more, even up to and including over the full length (or nearly the full length) of the two polynucleotide sequences being compared.

For sequence comparison and homology determination, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm can then be used to calculate the percent sequence identity for the test sequence(s) relative to the reference sequence, based on the designated program parameters.

Optimal alignment of sequences for comparison can be conducted, e.g., by the local homology algorithm (see e.g., Smith and Waterman, 1981), by the homology alignment algorithm (see e.g., Needleman and Wunsch, 1970), by the search similarity comparison method (see e.g., Pearson and Lipman, 1988), by computerized implementations of algorithms such as GAP, BESTFIT, FASTA, and TFASTA in the Wisconsin Genetics Software Package, Genetics Computer Group, Madison, Wis., USA, or by visual inspection.

One example of an algorithm that is suitable for determining percent sequence identity and sequence similarity is the BLAST algorithm (Altschul et al., 1990) and BLOSUM62 scoring matrix (see, e.g., Henikoff and Henikoff, 1989). Software for performing BLAST analyses is publicly available through the National Center for Biotechnology Information (www.ncbi.nlm.nih.gov/). In addition to calculating percent sequence identity, the BLAST algorithm also performs a statistical analysis of the similarity between two sequences (see, e.g., Karlin and Altschul, 1993). Another example of a useful sequence alignment algorithm is the PILEUP program, which creates a multiple sequence alignment from a group of related sequences using progressive, pairwise alignments. It can also plot a tree showing the clustering relationships used to create the alignment. PILEUP uses a simplification of the progressive alignment comparison method (see e.g., Feng and Doolittle, 1987). The method used is similar to the method described by Higgins and Sharp (1989).

### DEFINITIONS

As used herein the term “pro” refers to the AMH fragment or region consisting of amino acids 26 to 451 of human AMH.

The term “mature” refers to the AMH fragment or region consisting of amino acids 451-560 of human AMH.

The term “pro-mature” refers to full length AMH consisting of amino acids 26-560 of human AMH, and includes both uncleaved AMH (i.e., full length AMH existing as a single polypeptide chain) and cleaved-reassociated AMH in which the AMH polypeptide chain is cleaved but the pro and the mature fragments exist as a non-covalently associated complex.

The term “mid-pro” refers to the AMH fragment or region consisting of amino acids 230-451 of human AMH.

The term “N-ter-pro” refers to the AMH fragment or region consisting of amino acids 26-229 of human AMH.

The terms “about” and “approximately” as used herein, are interchangeable, and should generally be understood to refer to a range of numbers around a given number, as well as to all numbers in a recited range of numbers (e.g., “about 5 to 15” means “about 5 to about 15” unless otherwise stated). Moreover, all numerical ranges herein should be understood to include each whole integer within the range.

The term “e.g.,” as used herein, is used merely by way of example, without limitation intended, and should not be construed as referring only those items explicitly enumerated in the specification.

The terms “antibody” and “immunoglobulin.” as used herein, refer in a broad and general sense to any immunological binding agent, including, for example, polyclonal antibodies, monoclonal antibodies, as well as derivatives thereof. Antibodies may be assigned to one of five major classes: IgA, IgD, IgE, IgG, and IgM, depending on the type of constant domain within their heavy chains. Some of these major classes are further divided into subclasses or “isotypes,” including, for example, IgG1, IgG2, IgG3, IgG4, and such like. The heavy-chain constant domains that correspond to these different classes of immunoglobulins are termed α, δ, δ., γ and μ, respectively. Both the subunit structures as well as the three-dimensional configuration of the different classes of immunoglobulins are well-known to those of ordinary skill in the immunological arts.

As used herein the term “specifically binds” refers to an interaction between an antibody and its epitope having an affinity constant in the range of 10⁵ to 10¹⁰ LM⁻¹

As used herein, an “antigenic polypeptide” or an “immunogenic polypeptide” is a polypeptide which, when introduced into a vertebrate, reacts with the vertebrate's immune system molecules, i.e., is antigenic, and/or induces an immune response in the vertebrate, i.e., is immunogenic. Examples of antigenic and immunogenic polypeptides of the present invention include, but are not limited to, e.g., AMH, and in particular, dimeric mammalian forms of AMH, as well as epitopes or fragments or variants thereof.

As used herein, the term “carrier” is intended to include any solvent(s), dispersion medium, coating(s), diluent(s), buffer(s), isotonic agent(s), solution(s), suspension(s), colloid(s), inert(s) or such like, or a combination thereof, that is pharmaceutically acceptable for administration to the relevant animal. The use of one or more delivery vehicles for chemical compounds in general, and chemotherapeutics in particular, is well known to those of ordinary skill in the pharmaceutical arts. Except insofar as any conventional media or agent is incompatible with the active ingredient, its use in the diagnostic, prophylactic, and therapeutic compositions is contemplated. One or more supplementary active ingredient(s) may also be incorporated into, or administered in association with, one or more of the disclosed chemotherapeutic compositions.

As used herein, the term “epitope” refers to that portion of a given immunogenic substance that is the target of, i.e., is bound by, an antibody or cell-surface receptor of a host immune system that has mounted an immune response to the given immunogenic substance as determined by any method known in the art. Further, an epitope may be defined as a portion of an immunogenic substance that elicits an antibody response or induces a T-cell response in an animal, as determined by any method available in the art (see, for example, Geysen et al., 1984). An epitope can be a portion of any immunogenic substance, such as a protein, polynucleotide, polysaccharide, an organic or inorganic chemical, or any combination thereof. The term “epitope” may also be used interchangeably with “antigenic determinant” or “antigenic determinant site.”

As used herein, the term “expression” refers to the biological production of a product encoded by a coding sequence. In most cases, a polynucleotide (i.e., DNA) sequence, including the coding sequence, is transcribed to form a messenger-RNA (mRNA). The messenger-RNA is then translated to form a polypeptide product that has a relevant biological activity. The process of expression may involve further processing steps to the RNA product of transcription, such as splicing to remove introns, and/or post-translational processing of a polypeptide product.

The term “naturally occurring” as used herein as applied to an object refers to the fact that an object can be found in nature. For example, a polypeptide or polynucleotide sequence that is present in an organism that can be isolated from a source in nature and which has not been intentionally modified by the hand of man in a laboratory is naturally-occurring. As used herein, laboratory strains of rodents that may have been selectively bred according to classical genetics are considered naturally-occurring animals.

As used herein, a “heterologous” is defined in relation to a predetermined referenced gene sequence. For example, with respect to a structural gene sequence, a heterologous promoter is defined as a promoter that does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

As used herein, the term “homology” refers to a degree of complementarity between two polynucleotide or polypeptide sequences. The word “identity” may substitute for the word “homology” when a first nucleic acid or amino acid sequence has the exact same primary sequence as a second nucleic acid or amino acid sequence. Sequence homology and sequence identity can be determined by analyzing two or more sequences using algorithms and computer programs known in the art. Such methods may be used to assess whether a given sequence is identical or homologous to another selected sequence.

As used herein, “homologous” means, when referring to polypeptides or polynucleotides, sequences that have the same essential structure, despite arising from different origins. For example, AMH protein is highly conserved across mammalian species, and so is considered a homologous protein within that group. Typically, homologous proteins are derived from closely related genetic sequences, or genes. By contrast, an “analogous” polypeptide is one that shares the same function with a polypeptide from a different species or organism, but has a significantly different form to accomplish that function. Analogous proteins typically derive from genes that are not closely related.

The term “host cell” means a cell that contains one or more heterologous polypeptide(s) or and/or polynucleotide(s). Host cells can be prokaryotic cells such as E. coli, or eukaryotic cells such as yeast, insect, amphibian, avian or mammalian cells, including, for example, but not limited to, human cells. Exemplary host cells include, e.g., but are not limited to, African green monkey kidney cells (e.g., Vero cells) baby hamster kidney (BHK) cells, Chinese hamster ovary (CHO) cells, primary chick kidney (PCK) cells, Madin-Darby canine kidney (MDCK) cells, Madin-Darby bovine kidney (MDBK) cells, 293 cells, COS cells, and the like. Culture and propagation of such cells are within the purview of the person of ordinary skill in the art, particularly based on the guidance herein.

The terms “identical” or percent “identity,” in the context of two or more peptide sequences, refer to two or more sequences or subsequences that are the same or have a specified percentage of amino acid residues that are the same, when compared and aligned for maximum correspondence over a comparison window, as measured using a sequence comparison algorithm or by manual alignment and visual inspection.

As used herein, the terms “immunize” or “immunization” or similar terms refer to conferring the ability to mount a substantial immune response against a target antigen or epitope as it is expressed on a microbe or as the isolated epitope or antigen. These terms do not necessarily require complete immunity, but rather that an immune response be produced that is substantially greater than baseline. For example, a mammal is considered to be immunized against a target antigen, if the cellular and/or humoral immune response, preferably a substantial response, to the target antigen occurs following administration of AMH-specific epitopes, peptides, or polypeptides in accordance with the methods described herein.

As used herein, the term “immunological response” to a composition or vaccine denotes the development of a cellular and/or antibody-mediated immune response in the host animal. Generally, an immunological response includes (but is not restricted to) one or more of the following effects: (a) the production of antibodies; (b) the production of B cells; (c) the production of helper T cells; and/or (d) the production of cytotoxic T cells, that are specifically directed to a given antigen or hapten.

As used herein, the term “operably linked” refers to a linkage of two or more polynucleotides or two or more nucleic acid sequences in a functional relationship. A nucleic acid is “operably linked” when it is placed into a functional relationship with another nucleic acid sequence. For instance, a promoter or enhancer is operably linked to a coding sequence if it affects the transcription of the coding sequence. “Operably linked” means that the nucleic acid sequences being linked are typically contiguous, or substantially contiguous, and, where necessary to join two protein coding regions, contiguous and in reading frame. Since enhancers generally function when separated from the promoter by several kilobases and intronic sequences may be of variable lengths; however, some polynucleotide elements may be operably linked but not contiguous.

As used herein, the term “immunogenic” as used herein also refers to an amino acid sequence, or a portion of an amino acid sequence within a protein, polypeptide, or peptide that elicits an immunological response in a host animal.

As used herein, the term “immunogenic protein,” “immunogenic peptide,” or “immunogenic polypeptide” refers to proteins, peptides, and polypeptides that are immunologically active in the sense that once administered to the host, it is able to evoke an immune response of the humoral and/or cellular type directed against the protein. Preferably, the protein fragment is such that it has substantially the same immunological activity as the total protein. Thus, a protein fragment according to the invention comprises or consists essentially of or consists of at least one epitope or antigenic determinant. The term epitope relates to a protein site able to induce an immune reaction of the humoral type (B cells) and/or cellular type (T cells).

The phrases “isolated” or “biologically pure” refer to material that is substantially, or essentially, free from components that normally accompany the material as it is found in its native state. Thus, isolated AMH peptides, peptide epitopes, or AMH-specific antibodies in accordance with the invention preferably do not contain materials normally associated with the AMH peptides or epitopes in their in situ environment.

“Link” or “join” refers to any method known in the art for functionally connecting peptides, including, without limitation, recombinant fusion, covalent bonding, disulfide bonding, ionic bonding, hydrogen bonding, electrostatic bonding, and such like.

As used herein, the term “patient” (also interchangeably referred to as “host” or “subject”) refers to any host that can receive one or more of the pharmaceutical compositions disclosed herein. Preferably, the subject is a vertebrate animal, which is intended to denote any animal species (and preferably, a mammalian species such as a human being). In certain embodiments, a “patient” refers to any animal host including without limitation any mammalian host. Preferably, the term refers to any mammalian host, the latter including but not limited to, human and non-human primates, bovines, canines, caprins, cavines, corvines, epines, equines, felines, hircines, lapines, leporines, lupines, murines, ovines, porcines, ranines, racines, vulpines, and the like, including livestock, zoological specimens, exotics, as well as companion animals, pets, and any animal under the care of a veterinary practitioner. In particular embodiments, the mammalian patient is preferably human.

The phrase “pharmaceutically-acceptable” refers to molecular entities and compositions that preferably do not produce an allergic or similar untoward reaction when administered to a mammal, and in particular, when administered to a human. As used herein, “pharmaceutically acceptable salt” refers to a salt that preferably retains the desired biological activity of the parent compound and does not impart any undesired toxicological effects. Examples of such salts include, without limitation, acid addition salts formed with inorganic acids (e.g., hydrochloric acid, hydrobromic acid, sulfuric acid, phosphoric acid, nitric acid, and the like); and salts formed with organic acids including, without limitation, acetic acid, oxalic acid, tartaric acid, succinic acid, maleic acid, fumaric acid, gluconic acid, citric acid, malic acid, ascorbic acid, benzoic acid, tannic acid, pamoic (embonic) acid, alginic acid, naphthoic acid, polyglutamic acid, naphthalenesulfonic acids, naphthalenedisulfonic acids, polygalacturonic acid; salts with polyvalent metal cations such as zinc, calcium, bismuth, barium, magnesium, aluminum, copper, cobalt, nickel, cadmium, and the like; salts formed with an organic cation formed from N,N′-dibenzylethylenediamine or ethylenediamine; and combinations thereof.

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Throughout the disclosure, common one-letter and three-letter amino acid abbreviations have been employed following the conventional nomenclature in the art: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues described herein are preferred to be in the “l” isomeric form. However, residues in the “d” isomeric form may be substituted for any l-amino acid residue provided the desired properties of the polypeptide are retained. All amino-acid residue sequences represented herein conform to the conventional left-to-right amino-terminus to carboxy-terminus orientation.

As used herein, the term “contiguous amino acid sequence” refers to an amino acid sequence that is a portion of a larger amino acid sequence. The larger amino acid sequence can be any amino acid sequence disclosed herein. The contiguous amino acid sequence can contain a certain number of contiguous (i.e., consecutive) amino acids of the larger sequence. For example, the contiguous amino acid sequence can contain 3 or more, 5 or more, 10 or more, 15 or more, 20 or more, 25 or more, 30 or more, 35 or more, 40 or more, 45 or more, 50 or more, 60 or more, 70 or more, 80 or more, 90 or more, or 100 or more contiguous amino acids of the sequence.

“Protein” is used herein interchangeably with “peptide” and “polypeptide,” and includes both peptides and polypeptides produced synthetically, recombinantly, or in vitro and peptides and polypeptides expressed in vivo after nucleic acid sequences are administered into a host animal or human subject. The term “polypeptide” is preferably intended to refer to any amino acid chain length, including those of short peptides from about 2 to about 20 amino acid residues in length, oligopeptides from about 10 to about 100 amino acid residues in length, and longer polypeptides including from about 100 amino acid residues or more in length. Furthermore, the term is also intended to include enzymes, i.e., functional biomolecules including at least one amino acid polymer. AMH-specific polypeptides and proteins of the present invention also include polypeptides and proteins that are or have been post translationally modified, and include any sugar or other derivative(s) or conjugate(s) added to the backbone amino acid chain. All such AMH polypeptides, for example, and particularly those that are detectable by one or more of the disclosed anti-AMH dimer-specific antibody compositions are expressly contemplated to fall within the scope of the present disclosure.

The term “sequence,” when referring to amino acids, relates to all or a portion of the linear N-terminal to C-terminal order of amino acids within a given amino acid chain, e.g., polypeptide or protein; “subsequence” means any consecutive stretch of amino acids within a sequence, e.g., at least 3 consecutive amino acids within a given protein or polypeptide sequence. With reference to nucleotide chains, “sequence” and “subsequence” have similar meanings relating to the 5′ to 3′ order of nucleotides.

As used herein, the term “substantially homologous” encompasses sequences that are similar to the identified sequences such that antibodies raised against peptides having the identified sequences will react with peptides having the substantially homologous sequences. In some variations, the amount of detectable antibodies induced by the homologous sequence is identical to the amount of detectable antibodies induced by the identified sequence. In other variations, the amounts of detectable antibodies induced are substantially similar, thereby providing immunogenic properties. For example, “substantially homologous” can refer to at least about 75%, preferably at least about 80%, and more preferably at least about 85% or at least about 90% identity, and even more preferably at least about 95%, more preferably at least about 97% identical, more preferably at least about 98% identical, more preferably at least about 99% identical, and even more preferably still, at least substantially or entirely 100% identical (i.e., “invariant”).

The term “pharmaceutically acceptable salt” as used herein refers to a compound of the present disclosure derived from pharmaceutically acceptable bases, inorganic or organic acids. Examples of suitable acids include, but are not limited to, hydrochloric, hydrobromic, sulfuric, nitric, perchloric, fumaric, maleic, phosphoric, glycolic, lactic, salicyclic, succinic, toluene-p-sulfonic, tartaric, acetic, citric, methanesulfonic, formic, benzoic, malonic, naphthalene-2-sulfonic, trifluoroacetic and benzenesulfonic acids. Salts derived from appropriate bases include, but are not limited to, alkali such as sodium and ammonia. The production of one or more compositions comprising AMH polynucleotides, AMH polypeptides, or AMH antibodies in combination with one or more pharmaceutically-acceptable salt formulations, for example, is particularly contemplated to fall within the scope of the present application.

The term “recombinant” indicates that the material (e.g., a polynucleotide or a polypeptide) has been artificially or synthetically (non-naturally) altered by human intervention. The alteration can be performed on the material within or removed from, its natural environment or state. For example, a “recombinant nucleic acid” is one that is made by recombining nucleic acids, e.g., during cloning, DNA shuffling or other procedures, or by chemical or other mutagenesis; a “recombinant polypeptide” or “recombinant protein” is a polypeptide or protein which is produced by expression of a recombinant nucleic acid. The production of one or more recombinant AMH polynucleotides or recombinant AMH polypeptides, for example, is particularly contemplated to fall within the scope of the present application.

As used herein, the term “variant,” when used in the context of a polynucleotide or polypeptide, refers to a polynucleotide or polypeptide that differs from the wild-type polynucleotide or polypeptide by way of one or more nucleotide or amino acid mutation(s), substitution(s), insertion(s), deletion(s), or any combination thereof. Depending on the context, the term “mutant” is also used to denote a variation from the wild-type polynucleotide or polypeptide sequence. The mutant may arise naturally (i.e., the mutation therein occurring in nature), be random, spontaneous, or specifically engineered by the hand of man in a given polynucleotide or polypeptide sequence. Protein or peptide variants often typically include the exchange of one or more native amino acid(s) for (an) other amino acid(s) at one or more amino acid residues within the protein or peptide.

As used herein, the term “biological sample” refers to any fluid, tissue, cell, or other material obtained from a subject of either gender. In a preferred embodiment, the biological sample is a blood or serum sample obtained from a human female. In a preferred embodiment, the sample is whole blood provided as a dried spot on an adsorbent support, such as a paper disc. As used herein, the terms “serum” and “plasma” are used interchangeably. The biological sample can be obtained at any desired time, such as prior to, at the same time as, or following an ovulation induction procedure, or in conjunction with an effort to determine, diagnose, prognose, or treat any physiological or pathological condition.

As used herein, the term “substantially free” or “essentially free” in connection with the amount of a component preferably refers to a composition that contains less than about 10 weight percent, preferably less than about 5 weight percent, and more preferably less than about 1 weight percent of a compound. In preferred embodiments, these terms refer to less than about 0.5 weight percent, less than about 0.1 weight percent, or less than about 0.01 weight percent.

As used herein, “consisting essentially of” allows for the inclusion of materials or steps that do not materially affect the basic and novel characteristics of the claim. Any recitation herein of the term “comprising”, particularly in a description of components of a composition or in a description of elements of a device, can be exchanged with “consisting essentially of” or “consisting of”.

### 5. EXAMPLES

The following examples are included to demonstrate illustrative embodiments of the invention. It should be appreciated by those of ordinary skill in the art that the techniques disclosed in these examples represent techniques discovered to function well in the practice of the invention, and thus can be considered to constitute preferred modes for its practice. However, those of ordinary skill in the art should, in light of the present disclosure appreciate that many changes can be made in the specific embodiments which are disclosed and still obtain a like or similar result without departing from the spirit and scope of the invention.

### Example 1

### Antibody Production

Male AMH knockout mice on a C57bl/6 background were immunized with recombinant human AMH. An initial injection of 40 μg of recombinant human AMH was given to each mouse subcutaneously in Freund's Complete Adjuvant (MP Biomedical 0855828) and one booster injection of 40 μg in Freund's Incomplete Adjuvant (MP Biomedical 0855829) was given after one month. Tail bleeds were collected from the mice and titrations of the samples were screened on AMH coated plates with 10 μg/well. The highest-responding mouse was determined by color intensity (O.D. from 0-3, light yellow-dark yellow), and it was selected for final boosting. Final boosting was given one month after the booster injection date. For four consecutive days, the mouse received approximately 162 μg of recombinant human AMH, administered by intraperitoneal injection. On the fifth day, the mouse was sacrificed and the spleen was removed aseptically. Spleen cells were centrifuged, aliquoted and stored in liquid nitrogen.

### Example 2

### Antibody Screening

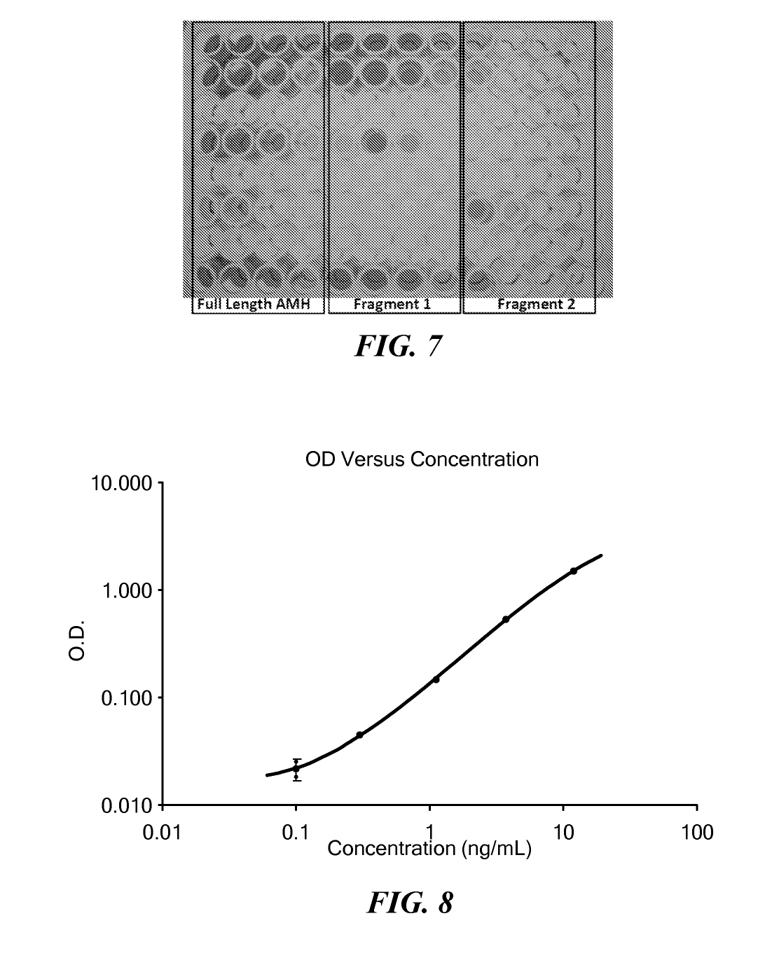

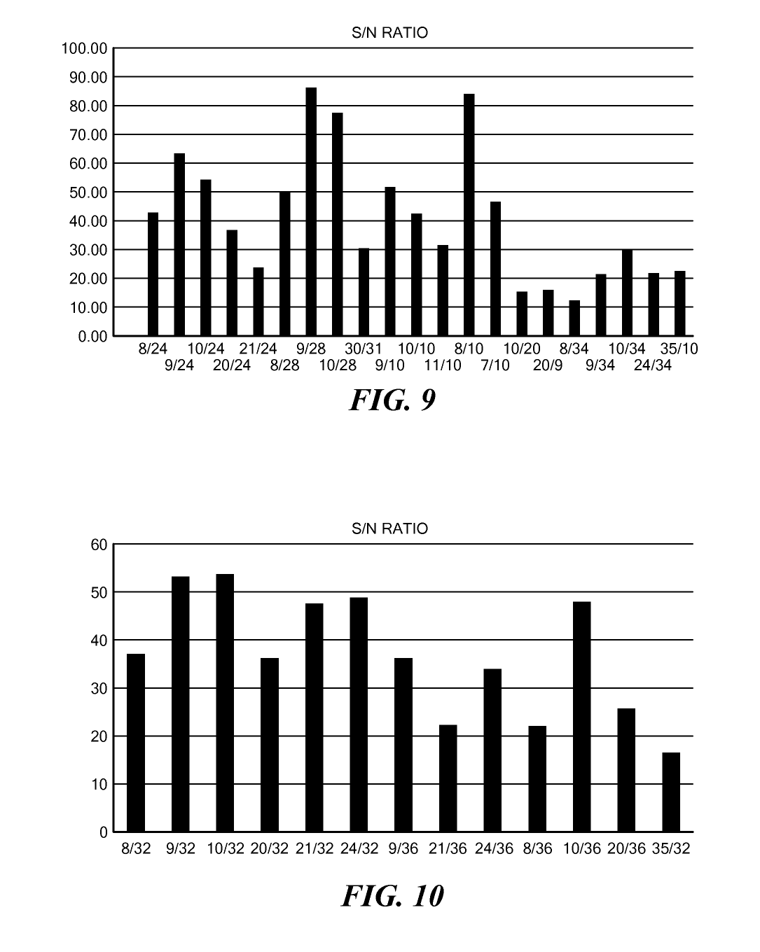

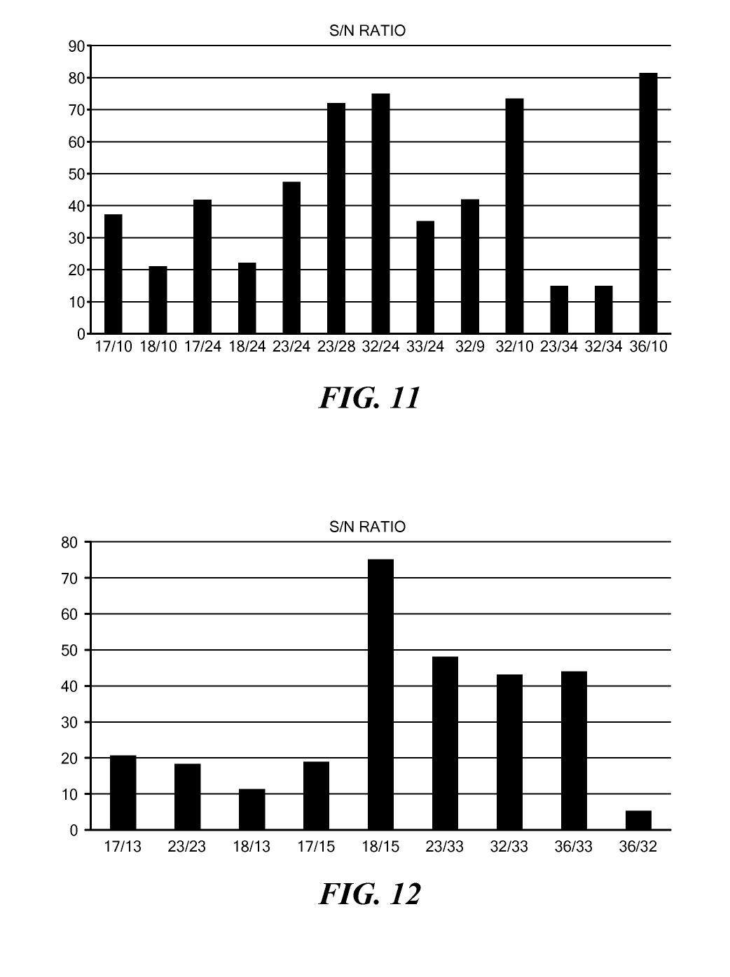

A fusion with SP2/0 cells was performed with one aliquot of the spleen cells. Fusions were initially screened on the immunogen, recombinant human AMH. Secondary screens were performed on the pro and mature fragments of AMH, to obtain more information on epitope location (FIG. 7). Positive clones were re-cloned, scaled up, and the secreted antibody purified on the GE AktaPrime Plus. After purification, the antibodies were paired and optimized based on the signal to noise ratio on full-length AMH, and the pro and mature fragments (See FIG. 9-FIG. 12).

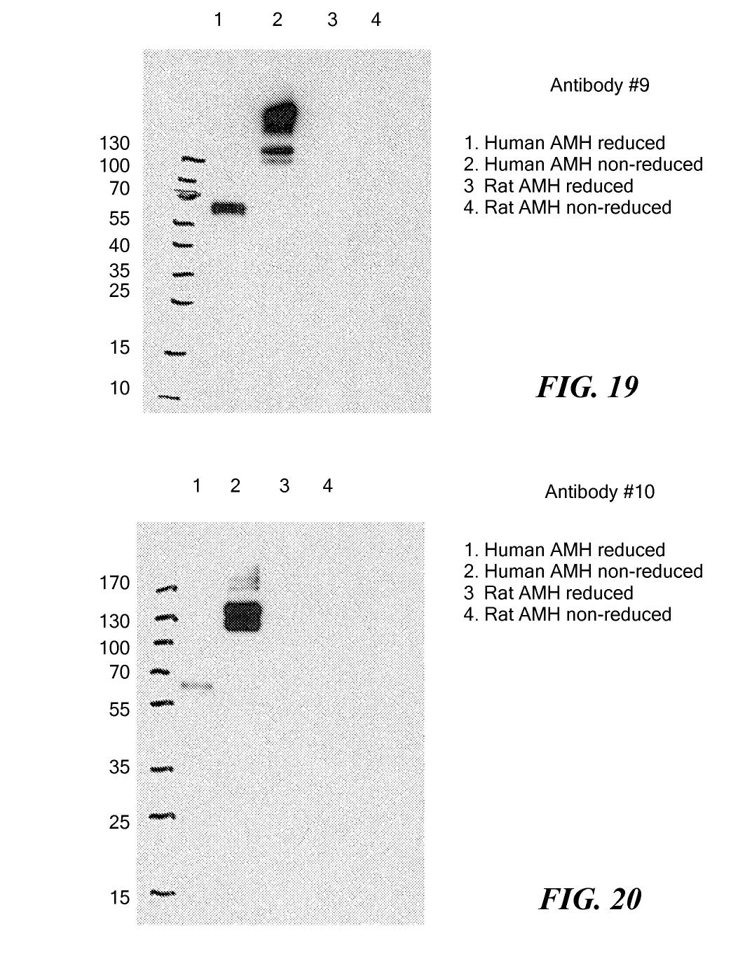

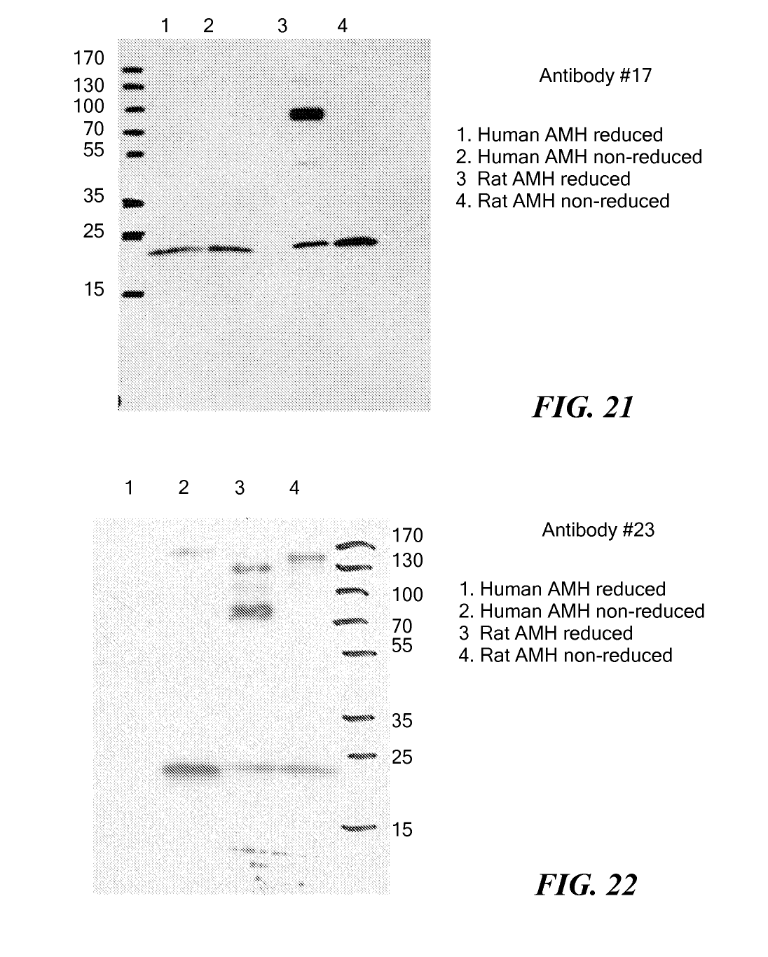

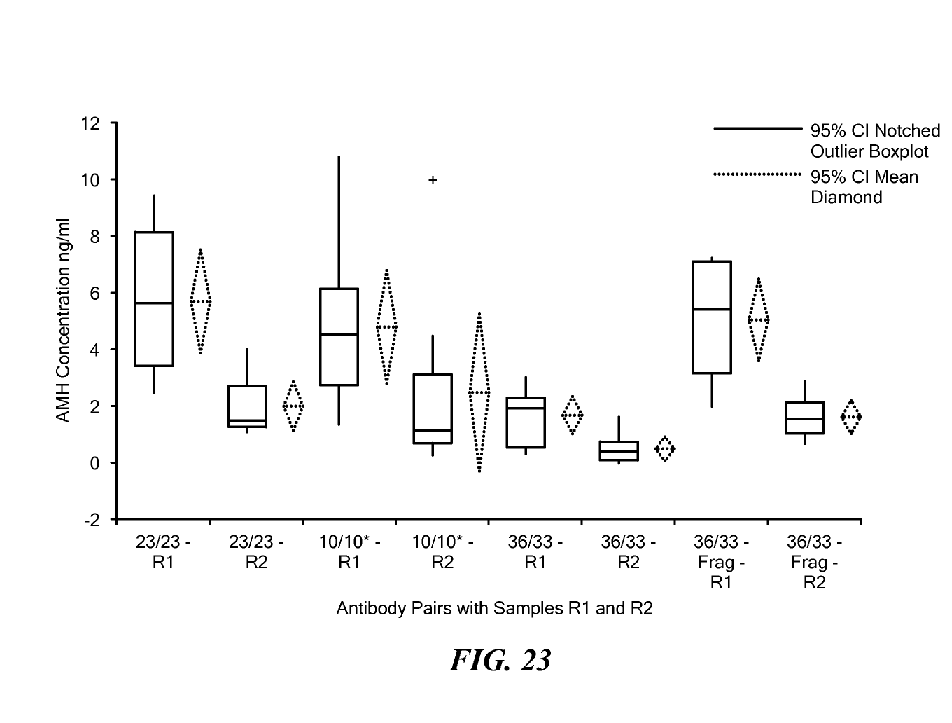

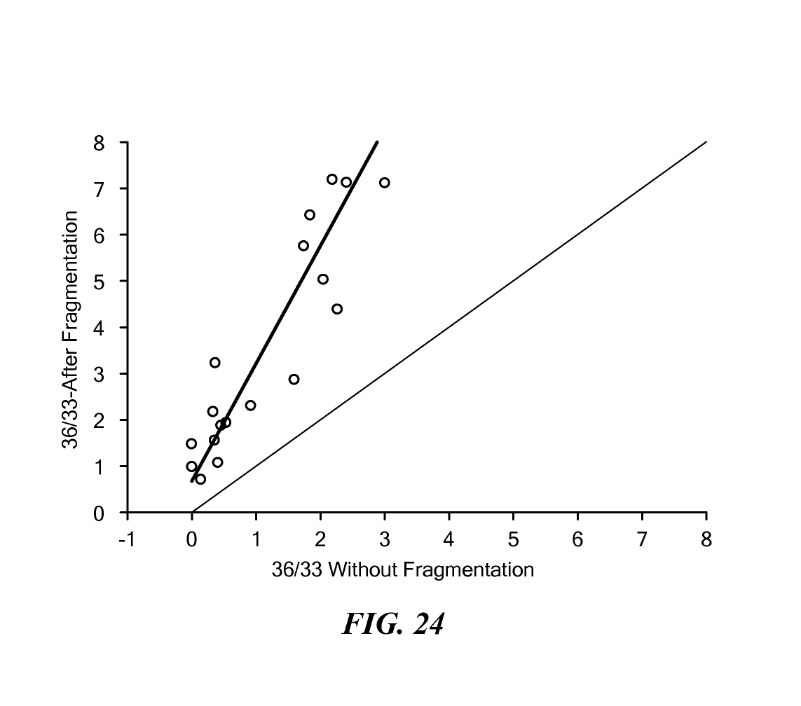

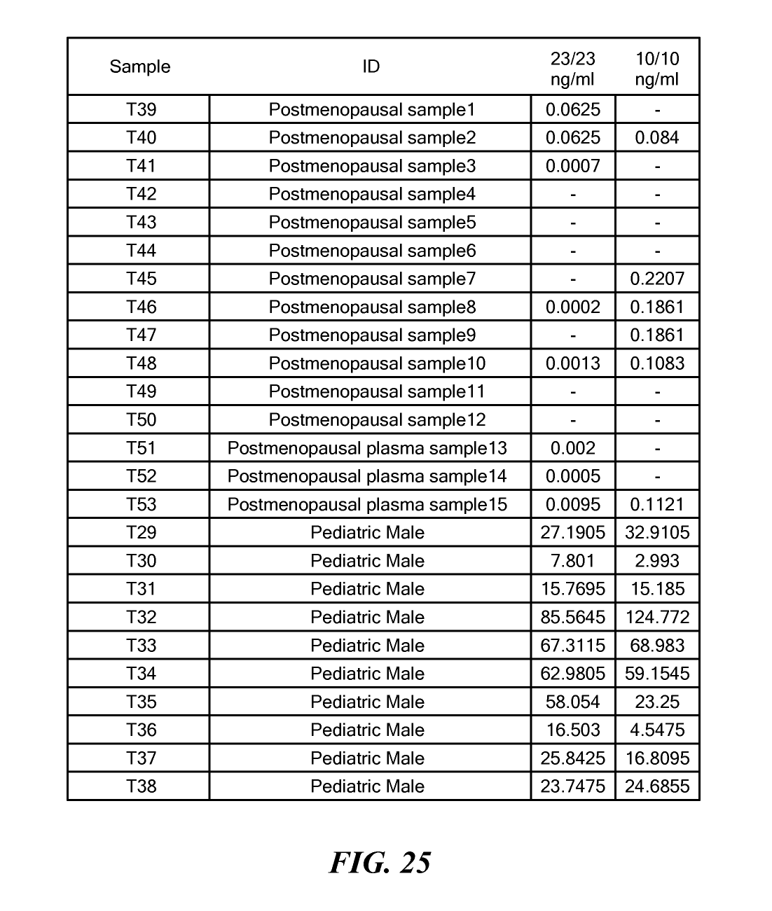

Western blots were performed to determine which antibodies bound to the pro and mature region of AMH. Antibodies #9 and #10 cleanly and specifically detected the human AMH pro region in both human reduced (monomeric) and non-reduced (dimeric) forms (see FIG. 19 and FIG. 20). Antibodies #17 and #23 detect both monomeric and dimeric mature regions for both human and rat AMH (See FIG. 21 and FIG. 22).

### Example 3

### Immunoassay

Coating of Microtiter Plates:

The monoclonal antibody used for coating was purified by Protein G (GE) affinity chromatography. Microtiter plates from Greiner Bio-One (Maybachstr. D-72636 Frickenhausen, Germany, cat: 705071, Greiner Bio-One North America. MONROE, N.C., USA) were coated with 100 μL/well of 10 μg/mL antibody in borate buffer overnight at room temperature. Excess antibody was removed by washing the plates once with 300 μL/well of neutral buffer. The plates were blocked with 200 μL/well of protein-based buffer and sucrose for 16-24 hours at room temperature (85% humidity). The blocking solution was aspirated, and the plates were dried in an environmental chamber at 34° C. for 4-5 hours. The plates were then packed in foil pouches with desiccant, labeled and stored at 2-8° C.

Biotinylation of Anti-AMH Monoclonal Antibody:

Purified monoclonal antibody was dialyzed in 0.1 M sodium borate buffer and biotinylated with NHS-Biotin. After two hours, the reaction mixture was dialyzed against 1 L of neutral buffer five times at 2-8° C. to remove excess biotin.

Calibration Curve:

Calibrators were made in animal sera using human AMH. Six point calibration (with the blank subtracted) were developed. The log of AMH concentration is plotted on the X-axis, the log of matched optical density on the Y-axis, and the curve is fit using cubic regression (FIG. 8).

### Example 4

### Assay Procedure

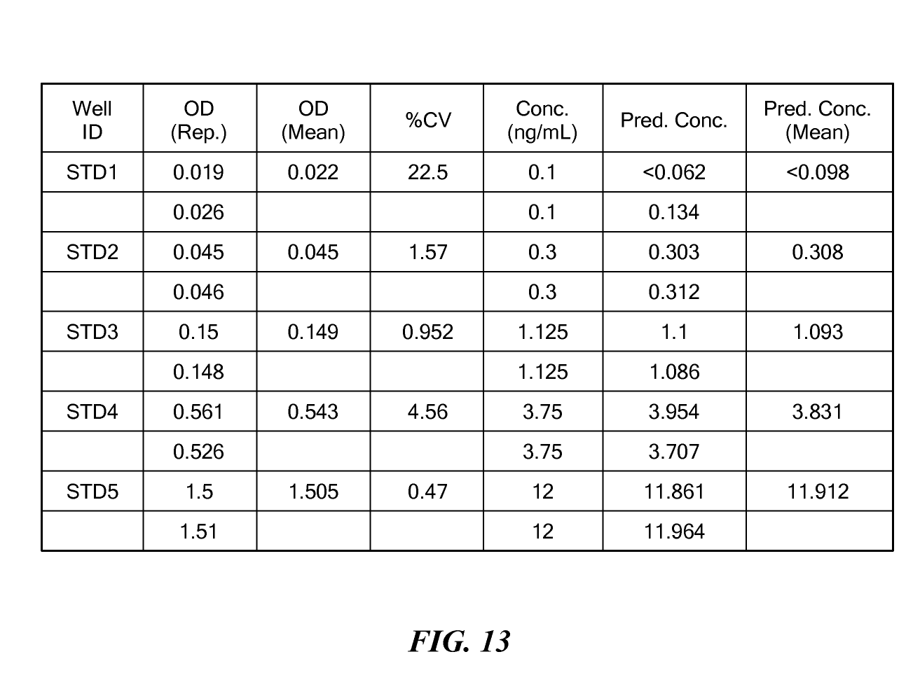

An AMH ELISA was tested as an enzymatically amplified two-site immunoassay. In the first step, 25 μL of calibrators (six vials, containing concentrations of approximately 0, 0.1, 0.3, 1.12, 3.75 and 12 ng/mL of human AMH in animal serum, controls and unknown samples, and 100 μL of assay buffer (protein-based buffer) were added to microtitration wells coated with the anti-AMH antibody. The wells were incubated while shaking on an orbital microplate shaker (RPM?) (Lab-Line Instrument Inc. USA) for 2 hours at room temperature (˜25° C.). Then, the plate was washed five times with Well Wash Versa microplate washer using phosphate buffer saline containing a non-ionic detergent (wash solution). In the second step, 100 μL of the AMH antibody-biotin conjugate (biotinylated anti-AMH antibody in protein-based buffer) was added to each well and then incubated on an orbital shaker for 1 hour at room temperature. Wells were then washed five times as described above. In the third step, 100 μL of streptavidin-labeled horseradish peroxidase enzyme conjugates in protein-based buffer was added to each well. Wells were incubated on an orbital shaker for an additional 30 minutes at room temperature, and then washed five times as described above. After the final wash step, 100 μL of tetramethylbenzidine (TMB) substrate solution was added to each well, and the samples were incubated on an orbital shaker for 10-12 min. The color formation was stopped by the addition of 100 μL stop solution (0.2 M H₂SO₄) to each well. The extent of enzymatic turnover of TMB was determined by dual wavelength absorbance measurement on a microplate ELISA reader (Gen 5; BioTek Instruments USA) at 450 nm (primary test filter) and 630 nm (primary reference filter). The absorbance measured was directly proportional to the concentration of AMH in the samples. Calibrators were used to plot a log-log cubic regression calibration curve of absorbance versus AMH concentration. The AMH concentrations in the samples were then interpolated from the calibration curve in FIG. 8. The data are shown in FIG. 13.

### Example 5

### Stability

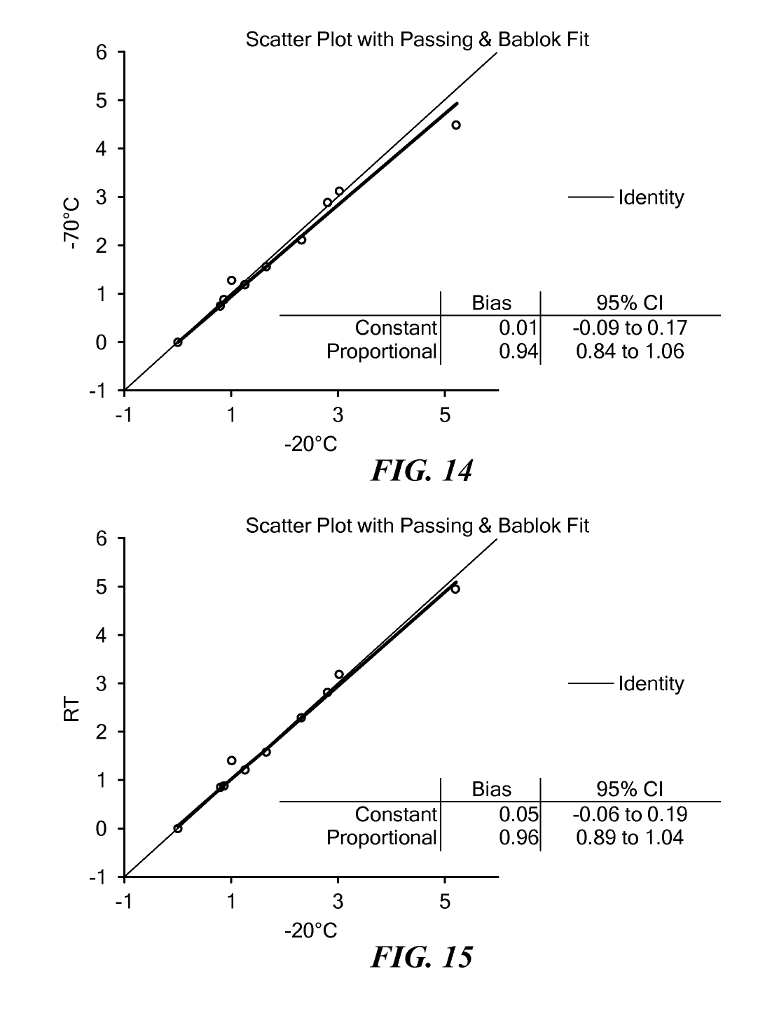

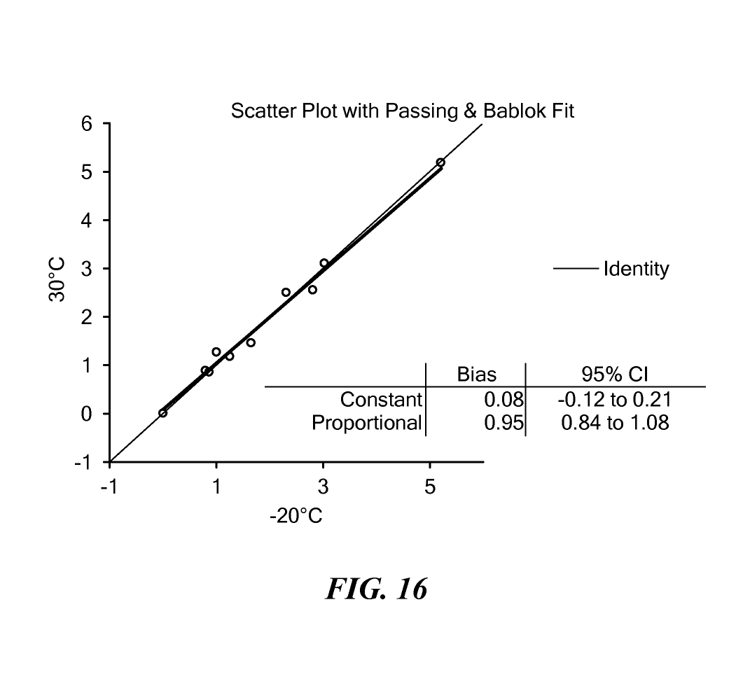

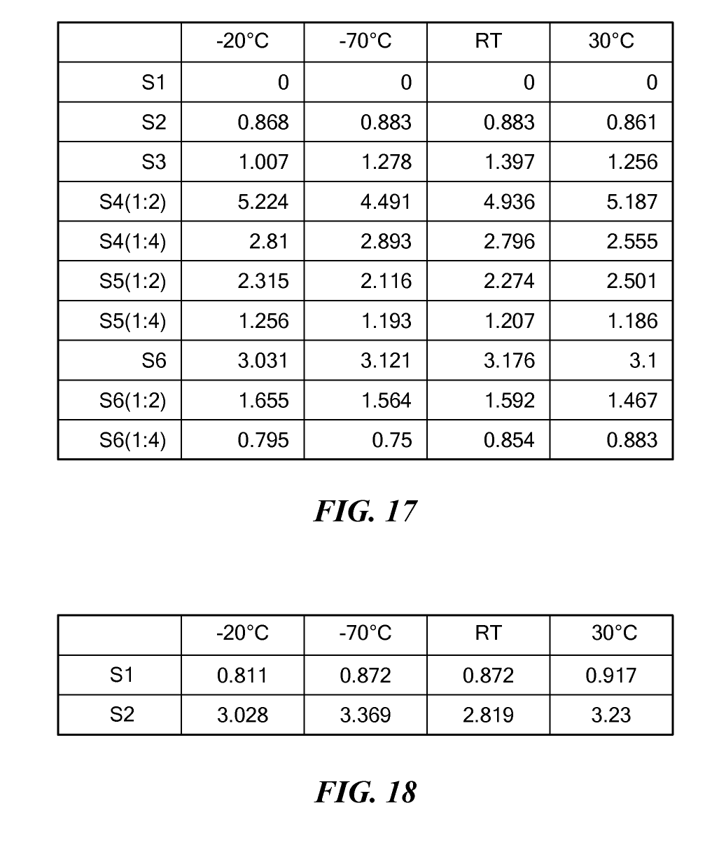

Six serum samples were aliquoted and incubated at −20° C., −70° C., room temperature (˜25° C.) and 30° C. for 24 hours (Day 1) and 3 days. The samples were tested in AMH SES assays for AMH recovery and reproducibility. On Day 1, 51, S2, and S3 were tested as “neat” samples. Samples S4 and S5 were diluted 1:2, 1:4 in a calibrator matrix. Sample S6 was tested “neat” and with a 1:2 or 1:4 dilution in a calibrator matrix. The recovery on the samples at different temperature conditions was compared (see FIG. 14, FIG. 15, FIG. 16 and FIG. 17).

On day three, 51, S2, S3 and S6 were tested neat in AMH ELISA SES assays. The recovery on the samples at different temperature conditions can be seen in FIG. 18. The results demonstrate that the measurement of AMH using the claimed assays is not impacted by sample storage conditions from −70° C. to 30° C. The data also suggests that the epitope of the antibodies in the claimed invention are not impacted by the proteolysis that has been an issue in the prior art.

As mentioned above, AMH is a very unstable molecule that can easily be cleaved by endogenous proteases in human blood. However, in the present invention in which monoclonal antibodies need the single relatively stable central epitope for sandwich formation, AMH displays significantly higher stability. See, for example, FIG. 14, FIG. 15, FIG. 16, and FIG. 17; the results of stability studies of AMH patients are presented. It is clear that neither temperature nor duration of incubation (24 hours-72 hours) affects the ability of the claimed assays to perform with specificity and sensitivity.

### Example 6

### Determining Different Forms of AMH in the Circulation

The assay in Example 4 was performed on several samples with several different antibody pairs. Antibody #23 was paired with itself in order to perform an “single epitope sandwich” SES assay. Antibody #23 was found to be directed toward the mature dimer of AMH using the protocol in Example 2. Thus, this pair created a mature-mature AMH assay. Antibody #9 was paired with itself in order to perform an SES assay. Antibody #9 was found to be directed toward the pro dimer of AMH using the protocol in Example 2. This pair created a pro-pro AMH assay. Antibody #17 was paired with antibody #13. Both antibody #17 and #13 were found to be directed toward the mature region of AMH using the protocol in Example 2. This pair created a mature-mature AMH assay. Antibody #24 was paired with antibody #32. Using the protocol in Example 2, antibody #24 was found to be directed toward the pro region of AMH, while antibody #32 was found to be directed toward the mature region of AMH. This pair created a pro-mature AMH assay. Finally, antibody #17 was paired with antibody #24, creating a mature-pro AMH assay.

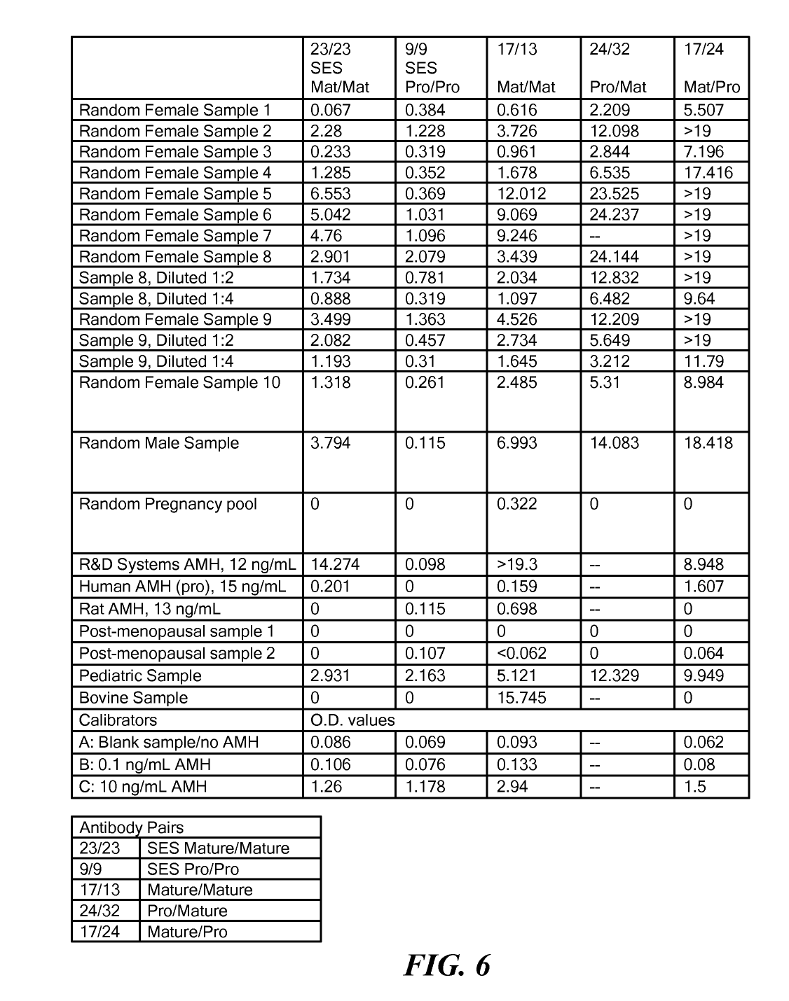

Each of the antibody pairs was used in assays to measure AMH in several different types of samples. Each assay was performed as described in Example 4 on identical human biological samples. In order to ensure their accuracy, all assays were calibrated using three controls—a) blank, b) 0.1 ng/mL AMH, and c) 10 ng/mL AMH. Assays were performed on identical samples, with the results being demonstrated in FIG. 6.

Several assays measured different amounts of AMH in the same sample. For example, all of the antibody pairs measured different amounts of AMH in sample number 5.

The SES mature-mature antibody pair 23/23 measured AMH at 6.553 ng/mL, while the mature-mature antibody pair 17/13 measured AMH at 12.012 ng/mL, and the pro-mature antibody pair 24/32 measured AMH at 23.525 ng/mL (See FIG. 6). The different assays are considered to be measuring different forms of AMH in circulation. The 23/23 SES mature-mature pair provided a measure of full-length and mature dimers of AMH. The 17/13 mature-mature pair measured full-length dimers and monomers, and mature dimers and monomers of AMH. Since it measures more forms of AMH in a sample than the SES 23/23 assay, it measures a greater value of AMH in the sample. The 24/32 pro-mature pair measured full-length dimers and monomers, and other full-length and monomeric fragments of AMH not recognizable by the 17/13 pair. Thus, there are isoforms that are recognizable by a pro-mature sandwich assay, but not a mature-mature sandwich assay.

### Example 7

### Epitope Mapping

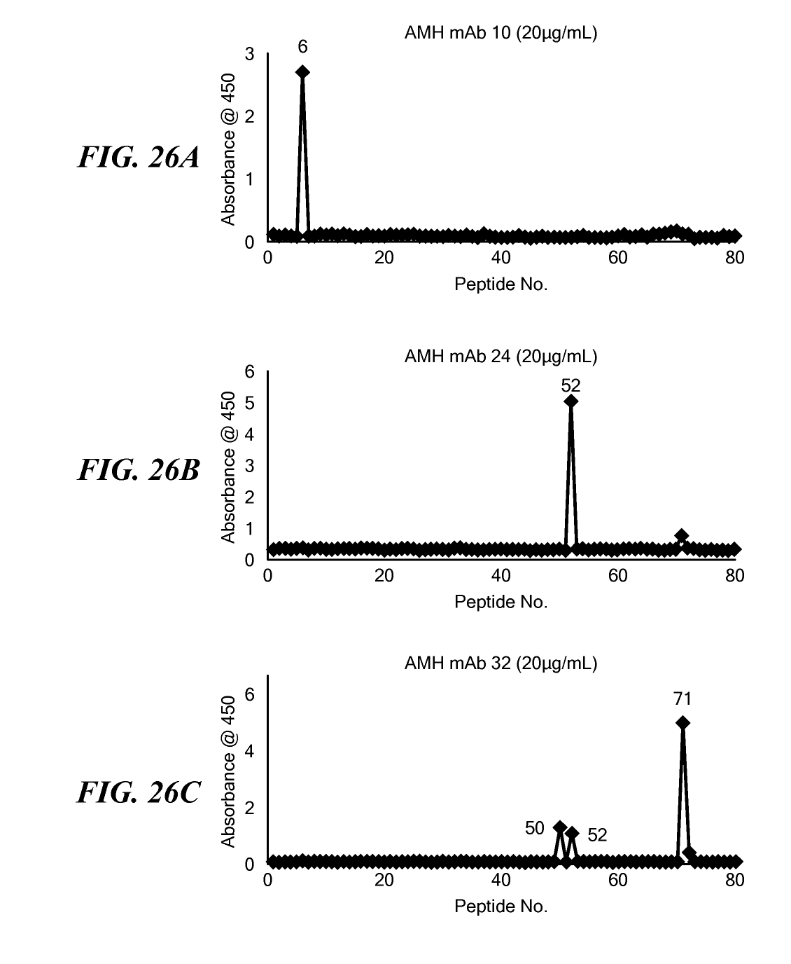

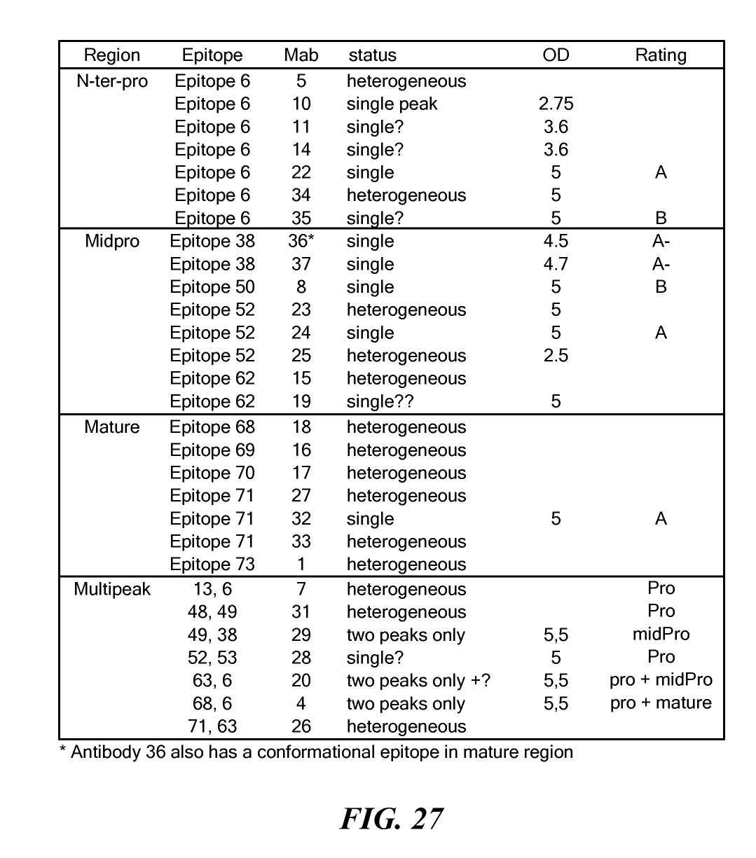

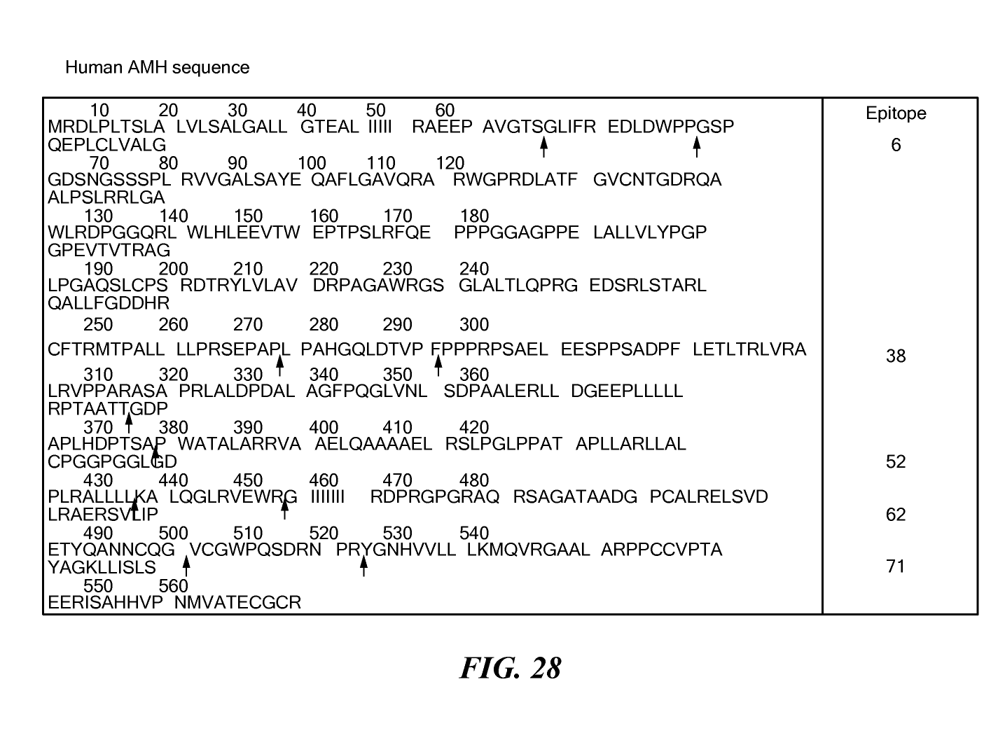

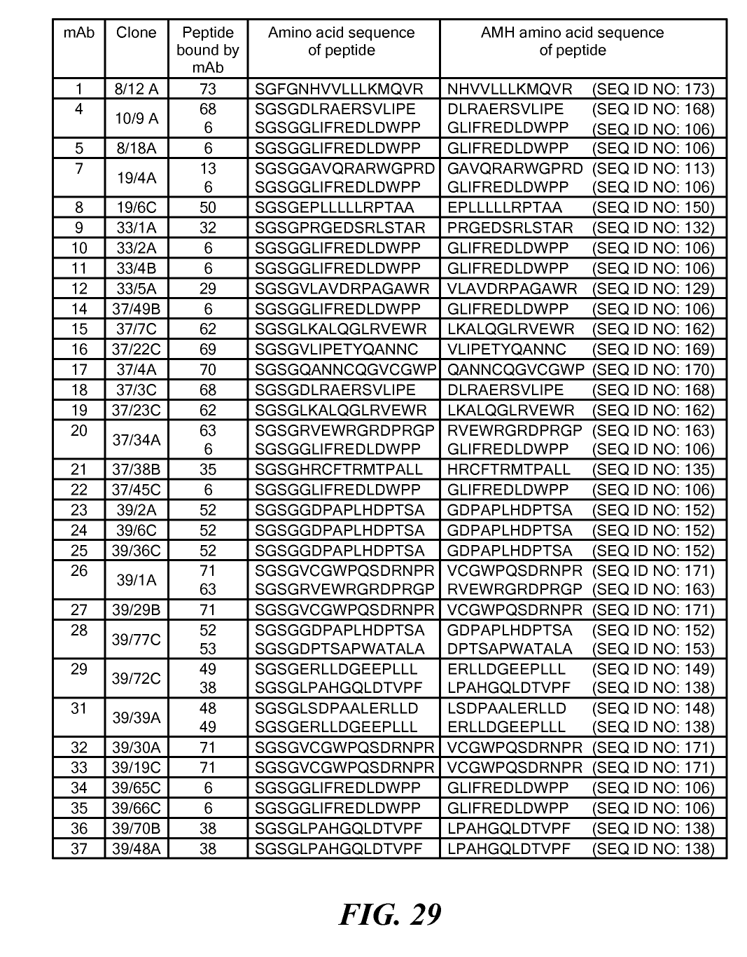

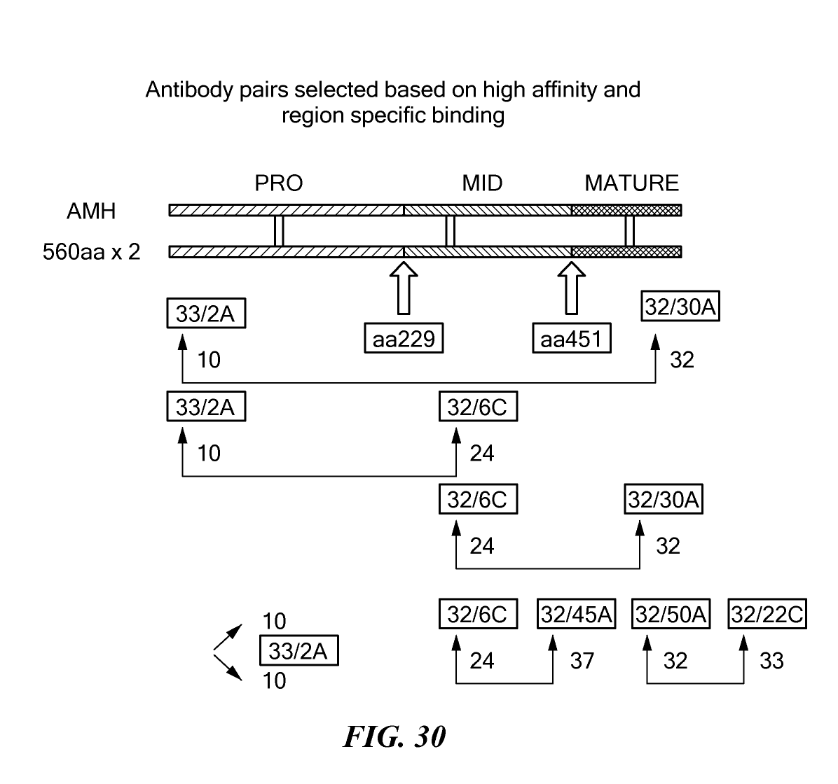

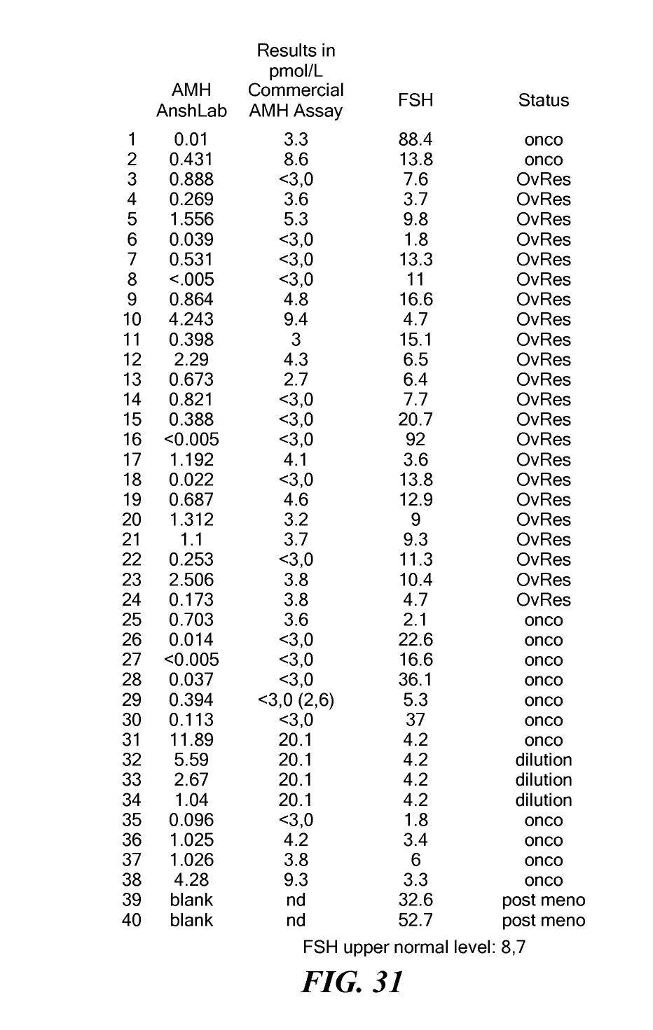

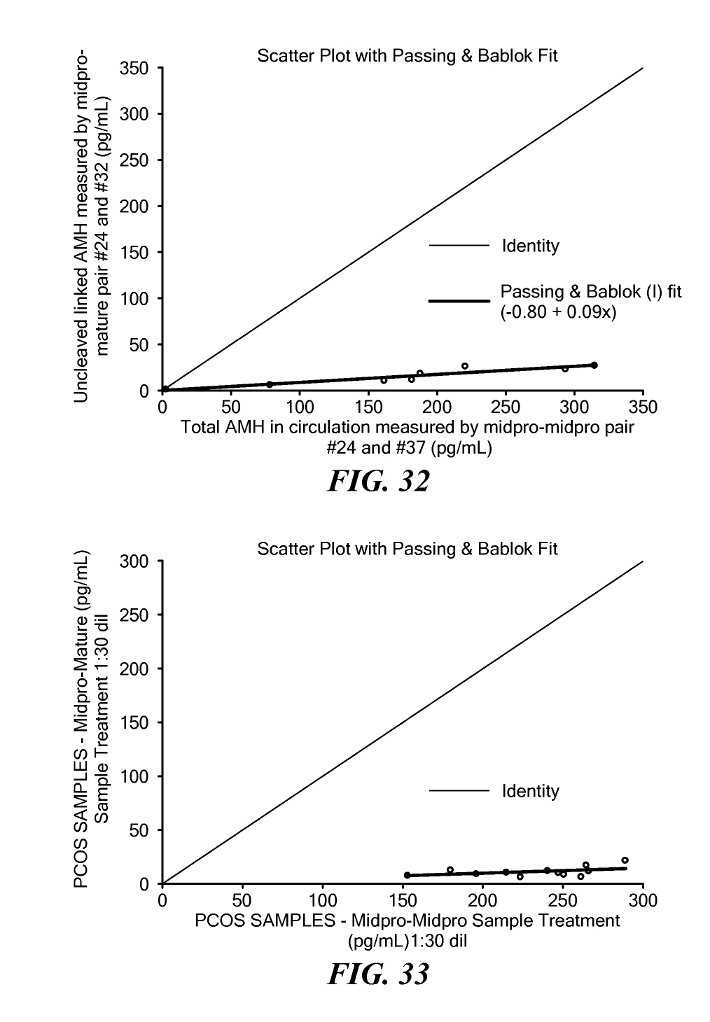

Epitope mapping was performed for a series of monoclonal antibodies generated against human AMH. The mAbs were tested using 80 synthetic peptides (see Table 1) covering the entire full-length sequence (amino acids 1-560) of human AMH. Each peptide contains a segment of 12 contiguous amino acids of the full length human AMH amino acid sequence. Binding of the antibodies to each peptide was determined using an indirect ELISA assay with the peptides bound to individual wells of a microtiter plate. The mAbs were assessed for homogeneity (i.e., whether a single peak was observed with a reasonable noise level), and the absorbance (optical density; OD) signal produced in the ELISA assay. The mAbs were sorted into “pro”, “mid”, and “mature” locations based on the known cleavage sites of AMH. FIGS. 26A-26C show representative results for antibodies binding to the pro (FIG. 26A), mid (FIG. 26B) and mature (FIG. 26C) regions of AMH. FIG. 27 summarizes results for a selection of epitopes in each of the pro, mid, and mature regions. FIG. 28 shows the position of selected epitopes within the AMH amino acid sequence. FIGS. 29A and 29B identify the epitopes bound by the indicated mAbs. FIG. 30 shows examples of pairs of mAbs that bind different regions of the AMH molecule, indicating how they can be combined to construct different sandwich ELISA assays to detect different forms of AMH. FIG. 31 shows a summary of clinical samples tested using a highly sensitive pair of antibodies (mAbs 24 and 32) in a sandwich ELISA to detect low picogram levels of AMH in human female patients. The results demonstrate that certain clinical conditions involving very low follicle number can be diagnosed using this “next generation AMH” assay which cannot be diagnosed using other AMH assays. The next generation AMH assay is linear down to at least 5 pg/mL, at least 3 pg/mL, or at least 1 pg/mL of AMH in human plasma.

TABLE 2
Human AMH Peptide Sequences Used for Epitope
Mapping.

| Peptide | | |
|---|---|---|
| no. | Peptide sequence | SEQ ID |
| 1 | SGSGMRDLPLTSLALV | SEQ ID NO: 21 |
| 2 | SGSGSLALVLSALGAL | SEQ ID NO: 22 |
| 3 | SGSGALGALLGTEALR | SEQ ID NO: 23 |
| 4 | SGSGTEALRAEEPAVG | SEQ ID NO: 24 |
| 5 | SGSGEPAVGTSGLIFR | SEQ ID NO: 25 |
| 6 | SGSGGLIFREDLDWPP | SEQ ID NO: 26 |
| 7 | SGSGLDWPPGIPQEPL | SEQ ID NO: 27 |
| 8 | SGSGPQEPLCLVALGG | SEQ ID NO: 28 |
| 9 | SGSGVALGGDSNGSSS | SEQ ID NO: 29 |
| 10 | SGSGNGSSSPLRVVGA | SEQ ID NO: 30 |
| 11 | SGSGRVVGALSAYEQA | SEQ ID NO: 31 |
| 12 | SGSGAYEQAFLGAVQR | SEQ ID NO: 32 |
| 13 | SGSGGAVQRARWGPRD | SEQ ID NO: 33 |
| 14 | SGSGWGPRDLATFGVC | SEQ ID NO: 34 |
| 15 | SGSGTFGVCNTGDRQA | SEQ ID NO: 35 |
| 16 | SGSGGDRQAALPSLRR | SEQ ID NO: 36 |
| 17 | SGSGPSLRRLGAWLRD | SEQ ID NO: 37 |
| 18 | SGSGAWLRDPGGQRLV | SEQ ID NO: 38 |
| 19 | SGSGGQRLVVLHLEEV | SEQ ID NO: 39 |
| 20 | SGSGHLEEVTWEPTPS | SEQ ID NO: 40 |
| 21 | SGSGEPTPSLRFQEPP | SEQ ID NO: 41 |
| 22 | SGSGFQEPPPGGAGPP | SEQ ID NO: 42 |
| 23 | SGSGGAGPPELALLVL | SEQ ID NO: 43 |
| 24 | SGSGALLVLYPGPGPE | SEQ ID NO: 44 |
| 25 | SGSGGPGPEVTVTRAG | SEQ ID NO: 45 |
| 26 | SGSGVTRAGLPGAQSL | SEQ ID NO: 46 |
| 27 | SGSGGAQSLCPSRDTR | SEQ ID NO: 47 |
| 28 | SGSGSRDTRYLVLAVD | SEQ ID NO: 48 |
| 29 | SGSGVLAVDRPAGAWR | SEQ ID NO: 49 |
| 30 | SGSGAGAWRGSGLALT | SEQ ID NO: 50 |
| 31 | SGSGGLALTLQPRGED | SEQ ID NO: 51 |
| 32 | SGSGPRGEDSRLSTAR | SEQ ID NO: 52 |
| 33 | SGSGLSTARLQALLFG | SEQ ID NO: 53 |
| 34 | SGSGALLFGDDHRCFT | SEQ ID NO: 54 |
| 35 | SGSGHRCFTRMTPALL | SEQ ID NO: 55 |
| 36 | SGSGTPALLLLPRSEP | SEQ ID NO: 56 |
| 37 | SGSGPRSEPAPLPAHG | SEQ ID NO: 57 |
| 38 | SGSGLPAHGQLDTVPF | SEQ ID NO: 58 |
| 39 | SGSGDTVPFPPPRPSA | SEQ ID NO: 59 |
| 40 | SGSGPRPSAELEESPP | SEQ ID NO: 60 |
| 41 | SGSGEESPPSADPFLE | SEQ ID NO: 61 |
| 42 | SGSGDPFLETLTRLVR | SEQ ID NO: 62 |
| 43 | SGSGTRLVRALRVPPA | SEQ ID NO: 63 |
| 44 | SGSGRVPPARASAPRL | SEQ ID NO: 64 |
| 45 | SGSGSAPRLALDPDAL | SEQ ID NO: 65 |
| 46 | SGSGDPDALAGFPQGL | SEQ ID NO: 66 |
| 47 | SGSGFPQGLVNLSDPA | SEQ ID NO: 67 |
| 48 | SGSGLSDPAALERLLD | SEQ ID NO: 68 |
| 49 | SGSGERLLDGEEPLLL | SEQ ID NO: 69 |
| 50 | SGSGEPLLLLLRPTAA | SEQ ID NO: 70 |
| 51 | SGSGRPTAATTGDPAP | SEQ ID NO: 71 |
| 52 | SGSGGDPAPLHDPTSA | SEQ ID NO: 72 |
| 53 | SGSGDPTSAPWATALA | SEQ ID NO: 73 |
| 54 | SGSGATALARRVAAEL | SEQ ID NO: 74 |
| 55 | SGSGVAAELQAAAAEL | SEQ ID NO: 75 |
| 56 | SGSGAAAELRSLPGLP | SEQ ID NO: 76 |
| 57 | SGSGLPGLPPATAPLL | SEQ ID NO: 77 |
| 58 | SGSGTAPLLARLLALC | SEQ ID NO: 78 |
| 59 | SGSGLLALCPGGPGGL | SEQ ID NO: 79 |
| 60 | SGSGGPGGLGDPLRAL | SEQ ID NO: 80 |
| 61 | SGSGPLRALLLLKALQ | SEQ ID NO: 81 |
| 62 | SGSGLKALQGLRVEWR | SEQ ID NO: 82 |
| 63 | SGSGRVEWRGRDPRGP | SEQ ID NO: 83 |
| 64 | SGSGDPRGPGRAQRSA | SEQ ID NO: 84 |
| 65 | SGSGAQRSAGATAADG | SEQ ID NO: 85 |
| 66 | SGSGTAADGPCALREL | SEQ ID NO: 86 |
| 67 | SGSGALRELSVDLRAE | SEQ ID NO: 87 |
| 68 | SGSGDLRAERSVLIPE | SEQ ID NO: 88 |
| 69 | SGSGVLIPETYQANNC | SEQ ID NO: 89 |
| 70 | SGSGQANNCQGVCGWP | SEQ ID NO: 90 |
| 71 | SGSGVCGWPQSDRNPR | SEQ ID NO: 91 |
| 72 | SGSGDRNPRYGNHVVL | SEQ ID NO: 92 |
| 73 | SGSGNHVVLLLKMQVR | SEQ ID NO: 93 |
| 74 | SGSGKMQVRGAALARP | SEQ ID NO: 94 |
| 75 | SGSGALARPPCCVPTA | SEQ ID NO: 95 |
| 76 | SGSGCVPTAYAGKLLI | SEQ ID NO: 96 |
| 77 | SGSGGKLLISLSEERI | SEQ ID NO: 97 |
| 78 | SGSGSEERISAHHVPN | SEQ ID NO: 98 |
| 79 | SGSGHHVPNMVATECG | SEQ ID NO: 99 |
| 80 | SGSGVPNMVATECGCR | SEQ ID NO: 100 |

TABLE 3
Amino Acid Sequences of AMH Peptides Containing
Epitopes of Human AMH.
Peptide

| no. | Peptide sequence | SEQ ID |
|---|---|---|
| 1 | MRDLPLTSLALV | SEQ ID NO: 101 |
| 2 | SLALVLSALGAL | SEQ ID NO: 102 |
| 3 | ALGALLGTEALR | SEQ ID NO: 103 |
| 4 | TEALRAEEPAVG | SEQ ID NO: 104 |
| 5 | EPAVGTSGLIFR | SEQ ID NO: 105 |
| 6 | GLIFREDLDWPP | SEQ ID NO: 106 |
| 7 | LDWPPGIPQEPL | SEQ ID NO: 107 |
| 8 | PQEPLCLVALGG | SEQ ID NO: 108 |
| 9 | VALGGDSNGSSS | SEQ ID NO: 109 |
| 10 | NGSSSPLRVVGA | SEQ ID NO: 110 |
| 11 | RVVGALSAYEQA | SEQ ID NO: 111 |
| 12 | AYEQAFLGAVQR | SEQ ID NO: 112 |
| 13 | GAVQRARWGPRD | SEQ ID NO: 113 |
| 14 | WGPRDLATFGVC | SEQ ID NO: 114 |
| 15 | TFGVCNTGDRQA | SEQ ID NO: 115 |
| 16 | GDRQAALPSLRR | SEQ ID NO: 116 |
| 17 | PSLRRLGAWLRD | SEQ ID NO: 117 |
| 18 | AWLRDPGGQRLV | SEQ ID NO: 118 |
| 19 | GQRLVVLHLEEV | SEQ ID NO: 119 |
| 20 | HLEEVTWEPTPS | SEQ ID NO: 120 |
| 21 | EPTPSLRFQEPP | SEQ ID NO: 121 |
| 22 | FQEPPPGGAGPP | SEQ ID NO: 122 |
| 23 | GAGPPELALLVL | SEQ ID NO: 123 |
| 24 | ALLVLYPGPGPE | SEQ ID NO: 124 |
| 25 | GPGPEVTVTRAG | SEQ ID NO: 125 |
| 26 | VTRAGLPGAQSL | SEQ ID NO: 126 |
| 27 | GAQSLCPSRDTR | SEQ ID NO: 127 |
| 28 | SRDTRYLVLAVD | SEQ ID NO: 128 |
| 29 | VLAVDRPAGAWR | SEQ ID NO: 129 |
| 30 | AGAWRGSGLALT | SEQ ID NO: 130 |
| 31 | GLALTLQPRGED | SEQ ID NO: 131 |
| 32 | PRGEDSRLSTAR | SEQ ID NO: 132 |
| 33 | LSTARLQALLFG | SEQ ID NO: 133 |
| 34 | ALLFGDDHRCFT | SEQ ID NO: 134 |
| 35 | HRCFTRMTPALL | SEQ ID NO: 135 |
| 36 | TPALLLLPRSEP | SEQ ID NO: 136 |
| 37 | PRSEPAPLPAHG | SEQ ID NO: 137 |
| 38 | LPAHGQLDTVPF | SEQ ID NO: 138 |
| 39 | DTVPFPPPRPSA | SEQ ID NO: 139 |
| 40 | PRPSAELEESPP | SEQ ID NO: 140 |
| 41 | EESPPSADPFLE | SEQ ID NO: 141 |
| 42 | DPFLETLTRLVR | SEQ ID NO: 142 |
| 43 | TRLVRALRVPPA | SEQ ID NO: 143 |
| 44 | RVPPARASAPRL | SEQ ID NO: 144 |
| 45 | SAPRLALDPDAL | SEQ ID NO: 145 |
| 46 | DPDALAGFPQGL | SEQ ID NO: 146 |
| 47 | FPQGLVNLSDPA | SEQ ID NO: 147 |
| 48 | LSDPAALERLLD | SEQ ID NO: 148 |
| 49 | ERLLDGEEPLLL | SEQ ID NO: 149 |
| 50 | EPLLLLLRPTAA | SEQ ID NO:150 |
| 51 | RPTAATTGDPAP | SEQ ID NO: 151 |
| 52 | GDPAPLHDPTSA | SEQ ID NO: 152 |
| 53 | DPTSAPWATALA | SEQ ID NO: 153 |
| 54 | ATALARRVAAEL | SEQ ID NO: 154 |
| 55 | VAAELQAAAAEL | SEQ ID NO: 155 |
| 56 | AAAELRSLPGLP | SEQ ID NO: 156 |
| 57 | LPGLPPATAPLL | SEQ ID NO: 157 |
| 58 | TAPLLARLLALC | SEQ ID NO: 158 |
| 59 | LLALCPGGPGGL | SEQ ID NO: 159 |
| 60 | GPGGLGDPLRAL | SEQ ID NO: 160 |
| 61 | PLRALLLLKALQ | SEQ ID NO: 161 |
| 62 | LKALQGLRVEWR | SEQ ID NO: 162 |
| 63 | RVEWRGRDPRGP | SEQ ID NO: 163 |
| 64 | DPRGPGRAQRSA | SEQ ID NO: 164 |
| 65 | AQRSAGATAADG | SEQ ID NO: 165 |
| 66 | TAADGPCALREL | SEQ ID NO: 166 |
| 67 | ALRELSVDLRAE | SEQ ID NO: 167 |
| 68 | DLRAERSVLIPE | SEQ ID NO: 168 |
| 69 | VLIPETYQANNC | SEQ ID NO: 169 |
| 70 | QANNCQGVCGWP | SEQ ID NO: 170 |
| 71 | VCGWPQSDRNPR | SEQ ID NO: 171 |
| 72 | DRNPRYGNHVVL | SEQ ID NO: 172 |
| 73 | NHVVLLLKMQVR | SEQ ID NO: 173 |
| 74 | KMQVRGAALARP | SEQ ID NO: 174 |
| 75 | ALARPPCCVPTA | SEQ ID NO: 175 |
| 76 | CVPTAYAGKLLI | SEQ ID NO: 176 |
| 77 | GKLLISLSEERI | SEQ ID NO: 177 |
| 78 | SEERISAHHVPN | SEQ ID NO: 178 |
| 79 | HHVPNMVATECG | SEQ ID NO: 179 |
| 80 | VPNMVATECGCR | SEQ ID NO: 180 |

### Example 8

### ELISA for Detection of Ultra-Low Concentrations of AMH

Based on measurements of AMH performed as described in Examples 3, 4 and 6 above, an ELISA kit for detection of ultra-low concentrations of AMH, the picoAMH (Anti-Müllerian hormone-enzyme linked immunuosorbent assay (ELISA) is described below. The kit provides materials for the quantitative measurement of ultra-low concentrations of AMH in human serum and other biological fluids. The picoAMH ELISA is a quantitative three-step sandwich type immunoassay designed to measure human pro-mature AMH complex. In the first step calibrators, controls and unknown samples are added to AMH antibody (capture antibody) coated microtiter wells and incubated. The AMH antibody used in the kit is specific to an epitope in the pro region of the molecule. After the first incubation and washing, the wells are incubated with biotinylated AMH antibody solution. The biotinylated AMH antibody may be specific to an epitope either in the pro or the mature region of AMH. After the second incubation and washing, the wells are incubated with streptavidin horseradish peroxidase conjugate (SHRP) solution. After the third incubation and washing step, the wells are incubated with substrate solution (TMB) followed by an acidic stopping solution. In principle, the AMH antibody-biotin conjugate binds to the solid phase antibody-antigen complex which in turn binds to the streptavidin-enzyme conjugate. The antibody antigen-biotin conjugate-SHRP complex bound to the well is detected by enzyme-substrate reaction. The degree of enzymatic turnover of the substrate is determined by dual wavelength absorbance measurement at 450 nm as primary test filter and 630 nm as reference filter. The absorbance measured is directly proportional to the concentration of AMH in the samples and calibrators.

The following materials are included in the kit.

1. A sample diluent containing 0 pg/mL AMH in protein based buffer and a non-mercury preservative, which may be stored unopened at 2-8° C.

2. A set of picoAMH calibrators, each calibrator contained in a separate vial. Each calibrator consists of a fixed amount of recombinant AMH protein lyophilized in the presence of a protein based buffer such that upon reconstitution with 1 ml of deionized water, the concentration of AMH in the vials range approximately between 10-1000 pg/mL. The vials may be store unopened at 2 to 8° C. Upon reconstitution of the AMH protein aliquots are made and frozen immediately for later use. Repeated freeze thaw are avoided. The recombinant AMH concentrations in calibrators are standardized to purified recombinant mature AMH preparation that is characterized by mass spectroscopy and optical density at 280 nm.

3. A calibrator control for each of high and low concentrations of AMH, contained in separate vials. Each vial contains AMH protein in protein based buffer and the preservative Pro-Clean 400 (Phoenix Ariz.)). The calibrator controls may be store unopened at 2 to 8° C. The contents of the vials are reconstituted in 1 mL deionized water, aliquot prepared and frozen immediately for later use. Repeated freeze thaws are avoided.

4. One stripholder, containing 12 strips and 96 microtitration wells with an anti-AMH antibody immobilized to the inside wall of each well.

5. Assay Buffer, which is a protein-based (BSA)-buffer with a nonmercury preservative.

6. Biotinylated anti-AMH antibody in protein based buffer with a non-mercury preservative.

7. Streptavidin-HRP (horseradish peroxidase) conjugate in a protein-based buffer and a non-mercury preservative.

8. TMB (tetramethyl benzidine)-100 TMB chromogen solution.

9. Stopping solution (0.2 M sulfuric acid).

10. Wash concentrate (buffered saline with a nonionic detergent; diluted 25-fold with deionized water prior to use).

Preferred sample types are serum or lithium heparin plasma. Samples may be stored at 4° C. if assayed within 24 hours; otherwise samples must be stored at −20° C. or −80° C. to avoid loss of bioactivity. Repeat freeze thaw of samples is avoided. Samples for analysis are not thawed for more than three times. A calibration curve is generated for each assay.

Reagents are prepared as follows. The picoAMH calibrators including the calibrators for high and low concentrations of AMH are each reconstituted with 1 mL deionized water. For sensitivity below 10 pg/mL the reconstituted calibrator for the lowest concentration of AMH may be further diluted with the sample diluent. The assay is carried out according to the instructions below.

1. Label the microtitration strips to be used.

2. Pipette 100 μL of the reconstituted calibrator, controls and unknowns to the appropriate wells.

3. Add 50 μL of the Assay Buffer to each well using a repeater pipette.

4. Incubate the plate, shaking at a fast speed (600-800 rpm) on an orbital microplate shaker, for 3 hrs. at room temperature.

5. Aspirate and wash each strip 5 times with wash solution using an automatic microplate washer.

6. Add 100 μL of the Biotinylated anti-AMH antibody to each well using a repeater pipette.

7. Incubate the plate, shaking at a fast speed (600-800 rpm) on an orbital microplate shaker, for 1 hr. at room temperature.

8. Aspirate and wash each strip 5 times with the wash solution using an automatic microplate washer.

9. Add 100 μL of the Streptavidin-HRP conjugate to each well using a repeater pipette.

10. Incubate the plate, shaking at a fast speed (600-800 rpm) on an orbital microplate shaker, for 30 minutes at room temperature.

11. Aspirate and wash each strip 5 times with the wash solution using an automatic microplate washer.

12. Add 100 μL of the TMB chromogen solution to each well using a precision pipette. Avoid exposure to direct sunlight.

13. Incubate the wells, shaking at 600-800 rpm on an orbital microplate shaker, for 8-12 min at room temperature. Visually monitor the color development to optimize the incubation time.

14. Add 100 μL of the stopping solution to each well using a precision pipette. Read the absorbance of the solution in the wells within 20 minutes, using a microplate reader set to 450 nm.

Results are calculated according to instructions below.

1. Calculate the mean optical density (OD) for each calibrator, control, or unknown.

2. Plot the log of the mean OD readings for each of the calibrators along the y-axis versus log of the AMH concentrations in pg/mL along the x-axis, using a cubic regression curve-fit.

3. Determine the AMH concentrations of the Controls and unknowns from the calibration curve by matching their mean OD readings with the corresponding AMH concentrations.

4. Any sample reading higher than the highest Calibrator should be appropriately diluted with the 0 pg/mL (sample diluent) and reassayed.

5. Any sample reading lower than the analytical sensitivity should be reported as such.

6. Multiply the value by a dilution factor, if required.

The reagents contained in the kit are optimized to measure AMH levels in human serum and lithium heparin plasma. Generally, for assays employing antibodies, the possibility exists for interference by heterophile antibodies in the samples (Kricka L. Interferences in immunoassays—still a threat, Clin Chem 2000; 46: 1037-1038.

The results are reported in ng/mL and can be converted to pmol/L using the conversion factor, 1 ng/ml=7.14 pmol/L.

Linearity of the kit was tested as follows:

Multiple dilutions of the three serum samples containing various AMH levels were diluted with Calibrator A/sample diluent. The % recovery on individual samples is represented in Table 4 below.

TABLE 4
Detection of AMH as a Function of Dilution.

| | | Expected | Observed | |
|---|---|---|---|---|
| | | Concentration | concentration | |
| Sample | Dilution factor | (pg · ml) | (pg/ml) | % Recovery |
| 1 | Neat | 346.88 | NA | NA |
| | 1:2 | 173.43 | 155.72 | 90% |
| | 1:4 | 86.72 | 79.79 | 92% |
| | 1:8 | 43.36 | 44.93 | 104% |
| | 1:16 | 21.68 | 24.33 | 112% |
| 2 | Neat | 1056.06 | Neat | NA |
| | 1:2 | 528.03 | 596.48 | 113% |
| | 1:4 | 264.01 | 270.36 | 102% |
| | 1:8 | 132.01 | 137.79 | 104% |
| | 1:16 | 66.0 | 75.55 | 114% |

Analytical Sensitivity: The analytical sensitivity in the assay as calculated by the interpolation of mean plus two standard deviation of 16 replicates of calibrator A (0 pg/mL) and calibrator B (7.9 pg/mL) is 0.63 pg/mL.

Analytical Specificity: The monoclonal antibody pair used in the assay is specific for human AMH and does not detect rat, mouse, porcine, equine, bovine, canine and ovine AMH. The antibody pair chosen does not cross reactivity to Inhibin A, Inhibin B, Activin A, Activin B, Activin AB, mature AMH and Pro AMH.

Interference: Potential interfering agents (hemoglobin, triglycerides and bilirubin) added at least at two times their physiological concentration to control sample, resulted in AMH concentration determinations to within ±10% of the control (no interfering agent added).

### Example 9

### Assay for Measuring Uncleaved and Cleaved-Reassociated Forms of AMH in the Circulation

After removal of the signal peptide (amino acids 1-18), and another seven amino acids following the signal peptide, the bulk of AMH undergoes processing to produce two fragments, one having amino acids 26-451, and the other having amino acids 452-560. The latter is the mature fragment of AMH. The fragment having amino acids 26-451 (i.e., pro AMH) may also undergo another processing step (Pepinsky et al. 1988) to produce two fragments, one having amino acids 26-229, referred to here as the N-ter-pro region, and the other having amino acids 230-451, referred to here as the midpro region. See Table 5.

TABLE 5
Human AMH Regions Resulting from Proteolytic Processing.

| AMH region | Amino acids |
|---|---|
| Pro | 26-451 |
| Mature | 452-560 |
| N-ter-pro | 26-229 |
| Midpro | 230-451 |
| Pro-mature | 26-560 |

Uncleaved and cleaved-reassociated forms of human AMH were measured using a sandwich ELISA carried out in the presence of the detergent SDS. Buffer containing the detergent was added to the sample prior to adding the sample to the capture antibody-coated microtiter wells. As an alternative, the detergent-containing buffer may be added directly to the wells prior to adding the sample. Final detergent concentration was 1%. Treatment with the detergent separated the midpro fragment (where there is a cleavage between amino acids 229 and 230 of AMH), or the pro fragment of AMH, from the mature fragment of AMH. Uncleaved AMH (the covalently linked form of AMH) was not affected by the addition the detergent.

Two types of ELISA were used to measure uncleaved (i.e., full length AMH having the pro and mid regions covalently attached) and cleaved AMH (i.e., full length AMH cleaved between the pro and mature regions, typically reassociated to form a non-covalently associated pro-mature dimer, also referred to here as “cleaved-reassociated” AMH). One sandwich ELISA used an antibody pair in which one antibody binds to an epitope in the midpro region (or an antibody that binds in the pro region in cases where there is no cleavage between amino acids 229 and 230 of AMH), and the other antibody binds to an epitope in the mature region; this yielded a measure of uncleaved AMH. Another sandwich ELISA used an antibody pair in which both antibodies bind to an epitope in the midpro region (or bind in the pro region in cases where there is no cleavage between amino acids 229 and 230 of AMH), or in which each antibody of the pair binds to epitopes in the mature region; this yielded a measure of total AMH (sum of uncleaved and cleaved-reassociated forms). Subtracting the amount of uncleaved AMH from the amount of total AMH produced a measure of the amount of the cleaved-reassociated from of AMH.

Some examples of antibody pairs that detect epitopes are shown in FIG. 30. These pairs yield a measure of either total AMH or uncleaved AMH, as follows (unless otherwise specified herein, the first named antibody of a pair is the capture antibody and the second is the detection antibody):
(1) Antibody #24 (midpro region specific) for capture and antibody #32 (mature region specific) for detection. This pair measures uncleaved AMH.
(2) Antibody #24 (midpro region specific) for capture and antibody #37 (midpro region specific) for detection. This pair measures total AMH.
(3) Antibody #36 (mature region specific) for capture and antibody #33 (mature region specific) for detection. This pair measures total AMH.
(4) Antibody #9 (pro region specific) for capture and antibody #10 (pro region specific) for detection. This pair measures total AMH.

A sandwich ELISA was performed using the antibody pair, #24 (midpro) and #32 (mature) in samples taken from eight human subjects (male and female). Another sandwich ELISA was performed using the antibody pair #24 (midpro) and #37 (midpro). The data were used to calculate the percent of uncleaved and cleaved-reassociated forms of AMH in the samples. The results are shown in Table 6. The range of AMH concentration measured was from 2.3040 pg/ml to 314.7030 pg/ml. Various statistical parameters of the measurement are also described in Table 6.

TABLE 6
Measurement of AMH Forms in Circulation in Normal Males/Females.

| | | | | Midpro- | Midro- | Uncleaved | Cleaved- |
|---|---|---|---|---|---|---|---|
| | | | | Mature | Midpro | AMH in | reassociated |
| Sample | Sample | | | 24/32 | 24/37 | circulation | AMH in |
| No | ID | Gender | Age | (pg/mL) | (pg/mL) | (%) | circulation (%) |
| 1 | 635 | M | 31 | 27.50 | 314.70 | 8.74 | 91.26 |
| 2 | 639 | F | 21 | 18.99 | 187.75 | 10.11 | 89.88 |
| 3 | 648 | M | 55 | 12.12 | 181.48 | 6.67 | 93.32 |
| 4 | 658 | M | 27 | 23.49 | 293.47 | 8.00 | 92.00 |
| 5 | 659 | F | 40 | 6.50 | 77.98 | 8.34 | 91.65 |
| 6 | 671 | M | 46 | 11.17 | 161.41 | 6.92 | 93.08 |
| 7 | 702 | F | 35 | 26.57 | 220.48 | 12.05 | 87.95 |
| 8 | 759 | F | 68 | 1.814 | 2.30 | 0 | 100.00 |

Statistical Parameters

| | | |
|---|---|---|
| Number of samples | 8 | |
| Range of AMH | 2.304-314.7030 | |
| concentration (pg/ml) | | |
| Number of replicates for | 1 | |
| each antibody pair | | |
| | Bias | 95% confidence interval |
| Constant | −0.08 | −17.7 to 4.61 |
| Proportional | 0.09 | 0.06 to 0.19 |
| r² statistic | 0.98 | |
| 95% confidence interval | 0.87 to 1.00 (normal | |
| | approximation) | |
| t statistic | 11.02 | |
| DF | 6 | |
| 2-tailed p | <0.0001 | |
| | (t approximation) | |

Another sandwich ELISA was performed using the same antibody pairs as above (24-32 and 24-37) to measure the amounts of uncleaved and cleaved-reassociated forms of AMH in patients with PCOS (polycystic ovarian syndrome). The results are shown in Table 7. The data indicate that the forms of AMH are not linearly correlated in PCOS.

TABLE 7
Measurement of AMH in Patients with PCOS.

| | | | Midpro- | Midpro- | Midpro- | Midpro- | Uncleaved | Cleaved- |
|---|---|---|---|---|---|---|---|---|
| | Midpro- | Midpro- | Mature | Midpro | Midpro | Midpro | AMH | reassociated |
| | Mature | Mature | (pg/mL) | 24/37 | 24/37 | 24/37 | in | AMH in |
| Sample | (pg/mL) | (pg/mL) | 1:120 | (pg/mL) | (pg/mL) | (pg/mL) | circulation | circulation |
| No. | 1:30 dil | 1:60 dil | dil | 1:30 dil | 1:60 dil | 1:120 dil | (%) | (%) |
| 1 | 7.032 | 3.559 | <1.814 | 261.469 | 131.653 | 66.043 | 2.69 | 97.31 |
| 2 | 9.245 | 5.654 | 2.839 | 196.033 | 104.727 | 55.042 | 4.72 | 95.28 |
| 3 | 12.392 | 6.021 | <1.971 | 240.309 | 132.654 | 64.228 | 5.16 | 94.84 |
| 4 | 17.773 | 9.708 | 4.922 | 264.498 | 145.096 | 70.405 | 6.72 | 93.28 |
| 5 | 8.045 | 4.831 | 3.109 | 153.319 | 85.727 | 45.389 | 5.25 | 94.75 |
| 6 | 10.632 | 4.102 | 2.216 | 247.327 | 120.93 | 57.457 | 4.30 | 95.70 |
| 7 | 11.095 | 6.48 | 3.018 | 214.699 | 116.213 | 58.182 | 5.17 | 94.83 |
| 8 | 9.153 | 5.379 | 2.839 | 250.714 | 133.28 | 70.89 | 3.65 | 96.35 |
| 9 | 12.67 | 6.297 | 2.928 | 180.072 | 85.239 | 41.288 | 7.04 | 92.96 |
| 10 | 11.836 | 5.654 | 4.375 | 265.992 | 136.666 | 78.054 | 4.45 | 95.55 |
| 11 | 22.239 | 8.692 | 4.923 | 289.072 | 151.038 | 68.588 | 7.69 | 92.31 |
| 12 | 6.572 | 3.288 | 2.04 | 223.445 | 111.756 | 58.303 | 2.94 | 97.06 |

Statistical Parameters

| | | |
|---|---|---|
| Number of samples | 12 | |
| Range of AMH concentration | 153.319 to 289.0720 | |
| (pg/ml; x-axis) | | |
| Number of replicates for | 1 | |
| each antibody pair | | |
| | Bias | 95% confidence interval |
| Constant | −0.06 | −22.74 to 15.72 |
| Proportional | 0.05 | −0.02 to 0.15 |
| r² statistic | 0.36 | |
| 95% confidence interval | 0.27 to 0.78 (normal approximation) | |
| t statistic | 1.23 | |
| DF | 10 | |
| 2-tailed p | 0.2453 | |
| | (t approximation) | |

### Example 10

### Accuracy and Linearity of an Assay for Measuring AMH Isoforms

Accuracy and linearity of the assays for measuring AMH isoforms (Example 9) was determined using a set of negative controls, and by measuring AMH concentrations at different dilutions. The negative controls used were: midpro fragment, mature fragment, and cleaved-reassociated AMH (containing all of the N-ter pro, midpro, and the mature regions).

As expected, with the use of the 24-32 antibody pair (capture in midpro and detection in mature), each of the midpro fragment, the mature fragment, and the cleaved-reassociated AMH controls failed to generate a significant signal over background. The lack of a positive signal using these controls and the 24-32 antibody pair shows that the assay is effective in separating the cleaved-reassociated form of AMH into the N-ter-pro and midpro, and mature fragments of AMH.

Similarly, as expected, with the use of the 24-37 antibody pair (both capture and detection in the midpro region), the midpro fragment and the cleaved-reassociated controls generated a positive signal, and the mature fragment control failed to generate a signal over background.

Additionally, use of the antibody pair 36-33 (both capture and detection in the mature region) produced no positive signal with the midpro AMH fragment control, and produced positive signals with both mature AMH fragment control and the cleaved-reassociated form of AMH.

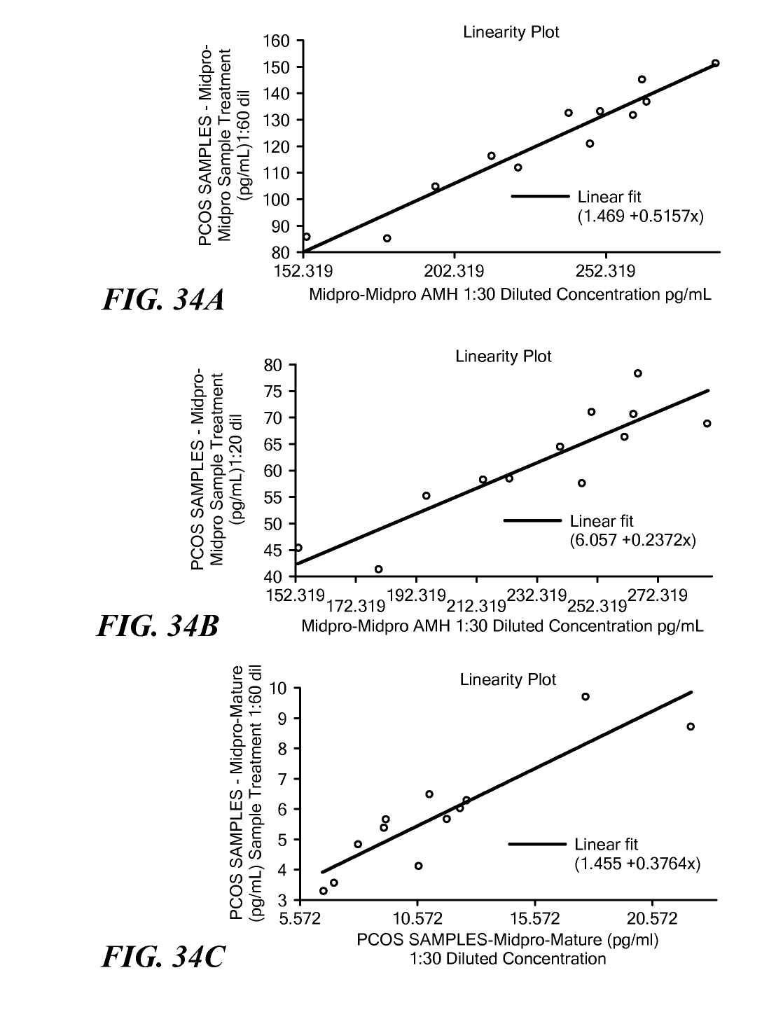

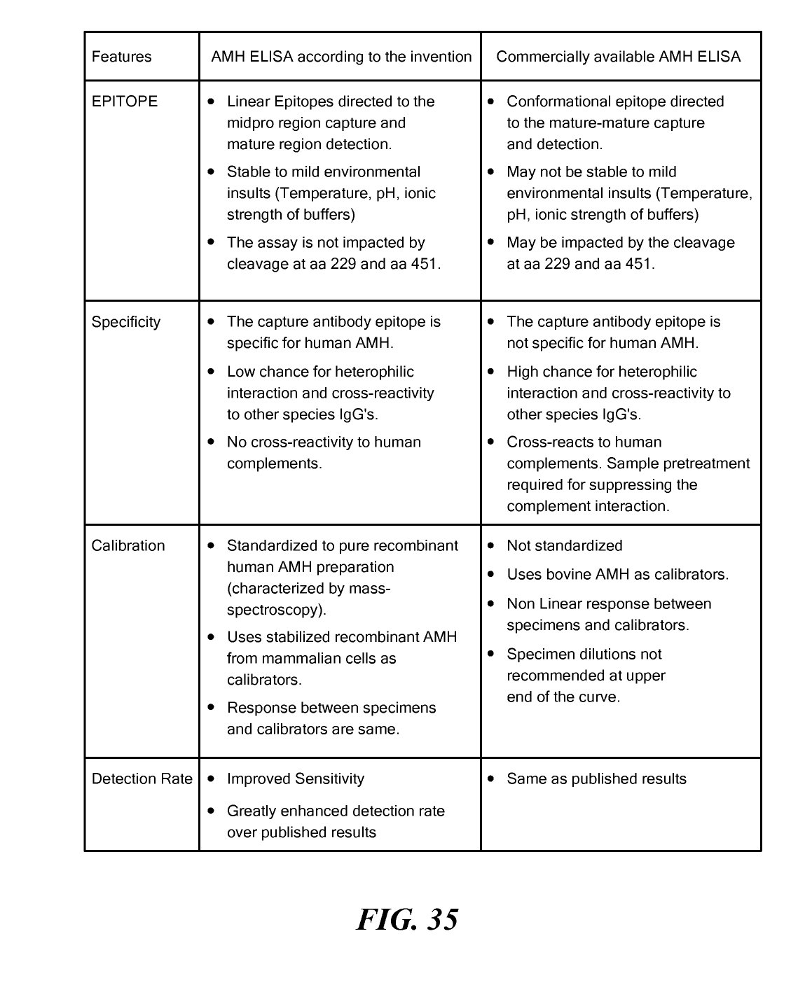

The linearity of the assay was determined, and the results are demonstrated by the graphs shown in FIGS. 34A-C. FIG. 34A shows a linearity plot of an assay performed at two dilutions, 1:30 and 1:60, using the antibody pair 24-37. Both antibodies bind to epitopes in the midpro region. The samples were taken from PCOS patients. The slope (0.5157) of the linear fit shows near perfect linearity for a two-fold dilution. FIG. 34B shows a linearity plot of an assay performed with PCOS patient samples at two dilutions, 1:30 and 1:120, also using the 24-37 antibody pair. The slope (0.2372) of the linear fit shows near perfect linearity for a four-fold dilution. FIG. 34C shows a linearity plot of an assay performed with PCOS patient samples at two dilutions, 1:30 and 1:60, using the 24-32 antibody pair. This assay is a midpro-mature assay, since the antibodies 24 and 37 bind in the midpro and mature regions, respectively. The slope (0.38 approximately) of the linear fit is consistent with a two-fold dilution.

### Example 11

### Determination of AMH in Dried Blood Samples

AMH was extracted from dried blood samples (DBS) that were spotted on filter paper discs, and its concentration determined using the assay described in Examples 8 and 9. Generally, a sample of blood was spotted on a filter paper disc and allowed to dry. AMH protein was extracted by shaking the disc containing the dried blood spot in a detergent containing buffer. Results of AMH concentration obtained from four dried samples, and corresponding serum controls (free of blood cells) are shown in Table 8.

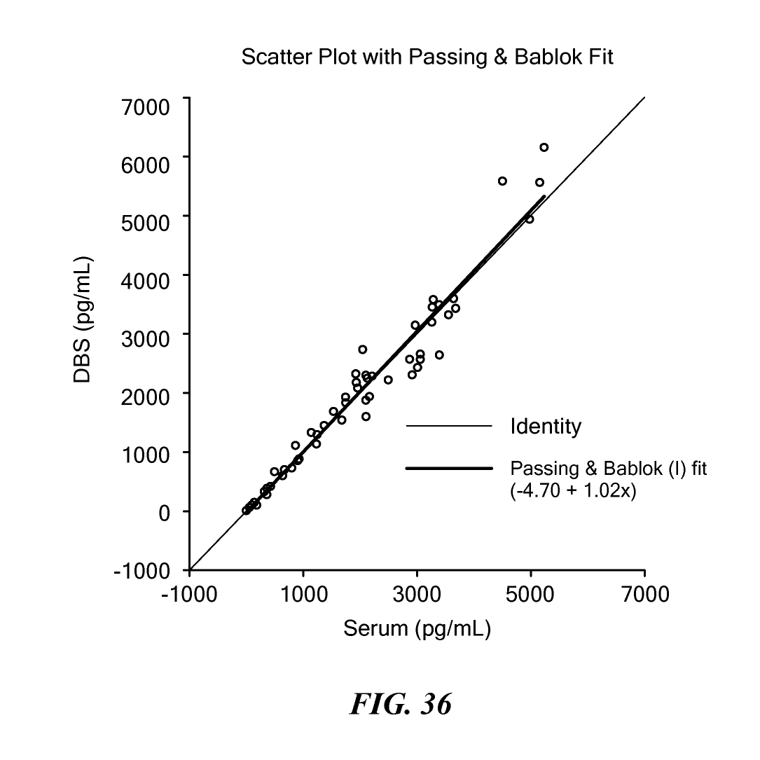

Additional results of AMH concentration determination from DBS, and the correlation between the measurements of AMH concentration obtained from DBS and those obtained from corresponding serum samples is shown in FIG. 36. A total of 65 samples were used in this test. The statistical parameters observed for this test are as follows: r² statistic 0.99; 95% confidence interval 0.98 to 0.99; t statistic 47.79; DF 63; and 2-tailed p<0.0001, (t approximation, corrected for ties).

The results indicate that the assay is effective to determine low levels of AMH in a dried blood sample. No interference from the presence of dried red blood cells, or other blood components present in the spots was observed even at the high sensitivity of detection (low picogram level) at which the assay was performed. Thus, the results also show that the assay could be carried out effectively without processing steps to separate blood cells from plasma, i.e., by directly processing whole blood, including whole dried blood. Details of the steps followed for preparing a dried blood sample and extraction of protein for AMH concentration determination are as follows:

DBS Specimen Collection Procedure: Blood was collected using capillary blood sampling, and applied on filter paper according the standard procedures. The first drop of blood was discarded. The next large drop of blood was applied to the surface of the first filter paper circle and allowed to fill and completely saturate the circle. Filling the circle by applying blood to both the front and the back of the paper was avoided. As a general practice multiple circles, e.g. three, were filled with blood. The blood impregnated filter papers were dried at room temperature for 2 to 4 hours while keeping the circles in a horizontal position. It was observed that the accuracy of assay for determining AMH concentration was not affected by storing the filter paper with dried blood spots in a low permeability re-sealable pouch at 2-8° C. for up to 1 week, or frozen at −20° C. or lower for up to three months. Use of desiccant and vacuum pacing to protect from moisture was also found to be helpful.

DBS Extraction Procedure: Extraction of AMH from dried blood spots was performed on the same day prior to testing. Use of circles not completely filled and impregnated with blood, or having irregularly shaped spots, or not impregnated throughout, or having multiple spots, or not properly dried was avoided. Culture tubes for extraction of test dried blood samples were appropriately labeled. Filter paper discs (7.9 mm), impregnated with dried blood specimen were punched out and transferred into the labeled tubes using clean tweezers. Commercially available automated punchers may also be used for transfer. Next, 450 μL of detergent containing extraction buffer was added to each tube and vortexed. The tubes were placed in a tight fitting tube rack and incubated with shaking at a slow speed (400-450 rpm) at room temperature for 60 minutes. The liquid was transferred to another appropriately labeled tube. The blood extract thus obtained was used for AMH concentration determination.

Microtitration strips (containing the capture antibody) were appropriately labeled. Reconstituted calibrators (AMH with known concentration; 100 μL), and controls were pipetted into the appropriate wells of the microtitration strips. AMH/MIS Assay Buffer (50 μL) was added to the calibrators and the controls. Extracted DBS sample (150 μL) was added to the appropriate wells. The plate was incubated at room temperature with shaking (600-800 rpm) on an orbital microplate shaker for three hours. Each well was washed five times with wash solution using an automatic microplate washer. Next, 100 μL of the Antibody-Biotin Conjugate Ready to use (RTU; appropriate detection antibody) was added to each well. The plate was incubated at room temperature with shaking (600-800 rpm) for 1 hour. Each well was washed five times with the wash solution using an automatic microplate washer. Subsequently 100 μL of the Streptavidin-Enzyme Conjugate-RTU was added to each well. The plate was incubated at room temperature with shaking (600-800 rpm) for 30 minutes. Each well was washed with wash solution using an automatic microplate washer. Next 100 μL of the TMB chromogen solution was added to each well using a precision pipette, followed by incubation and shaking at 600-800 for 8-12 min at room temperature. Color development was monitored visually to optimize the incubation time. Then 100 μL of stopping solution was added to each well using a precision pipette and absorbance of the solution in the wells was measured within 20 minutes, using a microplate reader set to 450 nm.

TABLE 8
Measurement of AMH in Dried Blood Samples.

| Sample | DBS | Matched Serum | DBS | Matched Serum |
|---|---|---|---|---|
| ID | (pg/mL) | (pg/mL) | (pg/mm disc) | (pg/uL) |
| 1 | 494.26 | 1226.9 | 32.95 | 40.90 |
| 2 | 299.18 | 1097.8 | 19.95 | 36.59 |
| 3 | 191.11 | 812.83 | 12.74 | 27.09 |
| 4 | 29.89 | 172.79 | 1.99 | 5.76 |

| | | |
|---|---|---|
| AVERAGE | 16.91 | 27.59 |
| Ratio | 0.61 | 1.63 |

### REFERENCES

The following references, to the extent that they provide exemplary procedural or other details supplementary to those set forth herein, are specifically incorporated herein in their entirety by express reference thereto:
U.S. Pat. No. 5,359,033 (filed Jan. 25, 1989);
U.S. Pat. No. 7,897,350 (filed May 24, 2006);
Bath, L. E. et al., “Depletion of ovarian reserve in young women after treatment for cancer in childhood: detection by anti-Müllerian hormone, inhibin B and ovarian ultrasound,” Human Reprod., 18(11):2368-74 (November 2003);
Broer, S. L. et al., “Anti-Müllerian hormone predicts menopause: A long-term follow-up study in normoovulatory women,” J. Clin. Endocrinol. Metab., 96(8):2532-39 (August 2011);
Brown J. “Breaking symmetry in protein dimers: Designs and functions” Protein Science (2006), vol. 15: 1-13;
Catteau-Jonard, S. et al., “Changes in serum anti-Müllerian hormone level during low-dose recombinant follicular stimulating hormone therapy for anovulation in polycystic ovary syndrome,” J. Clin Endocirnol. Metab., 92(11):4138-4143 (November 2007);
Hagen, C. P. et al., “Serum levels of Anti-Müllerian Hormone as a marker of ovarian function in 926 healthy females from birth to adulthood and in 172 Turner syndrome patients,” J. Clin. Endocrinol. Metab., 95(11):5003-10 (November 2010);
Harlow, Sioban D. et al., “Executive summary of the stages of reproductive aging workshop +10: addressing the unfinished agenda of staging reproductive aging,” J. Clin. Endocrinol. Metab., 97(4):1159-1168 (April 2012);
Hudson, Peter L. et al., “An immunoassay to detect human Müllerian inhibiting substance in males and females during normal development,” J. Clin. Endocrinol. Metab., 70(1):16-22 (January 1990);
Kricka L. Interferences in immunoassays—still a threat. Clin Chem 2000; 46: 1037-1038;
Kumar, A. et al., “Development of a second generation anti-Müllerian hormone (AMH) ELISA,” J. Immunol. Methods, 362, (Issues 1-2):51-59 (October 2010);
Lee, M. et al., “Müllerian inhibiting substance: a gonadal hormone with multiple functions,” Endocrine Rev., 14(2):152-164 (April 1993);
Lee, M. et al., “Müllerian inhibiting substance in humans: normal levels from infancy to adulthood,” J. Clin. Endocrinol. Metab., 81(2):571-76 (February 1996);
Long, W. Q. et al., “Detection of minimal levels of serum anti-Müllerian hormone during follow-up of patients with ovarian granulosa cell tumor by means of a highly sensitive enzyme-linked immunosorbent assay,” J. Clin. Endocrinol. Metab., 85(2):540-44 (February 2000);
Nelson, S. M. et al., “The journey from the old to the new AMH assay: how to avoid getting lost in the values,” Reprod. BioMed. Online, 23(Issue 4):411-420 (October 2011);
Pepinsky, R. B., Sinclair, L. K., Chow, E. P., Mattaliano, R. J., Manganaro, T. F., Donahoe, P. K. and Cate, R. L. (1988) Proteolytic processing of Mullerian inhibiting substance produces a transforming growth factor-b-like fragment. J. Biol. Chem., 263, 18961-18964.
Rajpert-De Meyts, E. et al., “Expression of anti-Müllerian hormone during normal and pathological gonadal development: association with differentiation of Sertoli and granulosa cells,” J. Clin. Endocrinol. Metab., 84(10):3836-3844 (October 1999);
Rey, R. A. et al., “Evaluation of gonadal function in 107 intersex patients by means of serum antimüllerian hormone measurement,” J. Clin. Endocrinol. Metab., 84(2):627-31 (February 1999);
Rustamov, Oybek et al., “Anti-Müllerian hormone: poor assay reproducibility in a large cohort of subjects suggests sample instability,” Human Reprod., 27 (10):3085-3091 (July 2012);
Segev, D. L. et al., “Müllerian inhibiting substance inhibits breast cancer cell growth through an NFκB-mediated pathway,” J. Biol. Chem., 275(37):28371-79 (September 2000);
Skaar, K. S. et al., “Proteolytically-activated, recombinant anti-Müllerian hormone inhibits androgen secretion, proliferation, and differentiation of spermatogonia in adult zebrafish testis organ cultures,”Endocrinol., 152(9):3527-40 (September 2011);
Van Disseldorp, J. et al., “Relationship of serum anti-Müllerian hormone concentration to age at menopause,” J. Clin. Endocrinol. Metab., 93(6):2129-34 (June 2008);
Visser, J. A. and Themmen, A. P. N., “Anti-Müllerian hormone and folliculogensis,” Mol. Cell. Endocrinol., 234 (Issues 1-2):81-86 (April 2005);
Wilson, C. et al., “Müllerian inhibiting substance requires its N-terminal domain for maintenance of biological activity, a novel finding within the transforming growth-factor-beta superfamily, Molec. Endocrinol., 7(2):247-57 (February 1993); and
Zec, I. et al., “Anti-Müllerian hormone: A unique biochemical marker of gonadal development and fertility in humans,” Biochemica Medica, 21(3):219-30, (October 2011).

All of the compositions and methods disclosed and claimed herein can be made and executed without undue experimentation in light of the present disclosure. While the compositions and methods of this invention have been described in terms of exemplary embodiments, it will be apparent to those of ordinary skill in the art that variations may be applied to the composition, methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those of ordinary skill in the art are deemed to be within the spirit, scope and concept of the invention as defined herein.

1. An isolated antibody that specifically binds to an epitope of human AMH contained in an amino acid sequence selected from the group consisting of: SEQ ID NO:106, SEQ ID NO:113, SEQ ID NO:150, SEQ ID NO:132, SEQ ID NO:129, SEQ ID NO:162, SEQ ID NO:163, SEQ ID NO:135, SEQ ID NO:152, SEQ ID NO:153, SEQ ID NO: 149, SEQ ID NO:138, SEQ ID NO:148, SEQ ID NO:173, SEQ ID NO:169, SEQ ID NO:170, SEQ ID NO:168, and SEQ ID NO:171.

2.-9. (canceled)

10. The antibody according to claim 1, wherein the antibody is a linear antibody, a chimeric antibody, a recombinant antibody or an antibody fragment selected from the group consisting of: Fv, Fab, F(ab′)₂, Fab′, dsFv, scFv, sc(Fv)₂, and diabody fragments.

11.-17. (canceled)

18. An immunodetection reagent comprising the antibody according to claim 1 linked to at least a first detectable label selected from a chemiluminescent agent, a colorimetric agent, an energy transfer agent, an enzyme, a fluorescent agent, and a radioisotope.

19. (canceled)

20. The immunodetection reagent according to claim 18 bound to a solid support, selected from a microtiter plate, a colloidal metal particle, an iron oxide particle, a latex particle, and a polymeric bead.

21.-24. (canceled)

25. A method of specifically detecting a dimeric form of human AMH, the method comprising:
performing a sandwich ELISA assay using a first antibody for capture and a second antibody for detection, wherein the first and second antibodies specifically bind to the same epitope of AMH, and wherein the epitope is contained in an amino acid sequence selected from the group consisting of: SEQ ID NO:132, SEQ ID NO:152, and SEQ ID NO:106.

26-27. (canceled)

28. A method of specifically detecting a monomeric form of human AMH, the method comprising:
performing an ELISA assay using an antibody that specifically binds to an epitope of AMH contained in an amino acid sequence selected from the group consisting of: SEQ ID NO:173, SEQ ID NO:106, SEQ ID NO:168, SEQ ID NO: 113.

29. A method of quantifying a dimeric form of human AMH in a sample, the method comprising:
performing a sandwich ELISA on the sample using a first antibody for capture and a second antibody for detection, wherein the first and second antibodies specifically bind to the same epitope of AMH, and wherein the epitope is contained in an amino acid sequence selected from the group consisting of: SEQ ID NO:132, SEQ ID NO:152, and SEQ ID NO:106;
measuring a detection signal generated by an agent conjugated to the second antibody; and
calculating the amount of dimeric human AMH in the sample by comparing the detection signal to a calibration curve correlating the amount of dimeric AMH to the detection signal.

30. (canceled)

31. The method according to claim 29, wherein dilution of a sample for quantification of AMH using the method yields substantially the same result, accounting for the dilution, when the sample is diluted to an AMH concentration anywhere in the range from about 20,000 pg/mL to about 1 pg/mL.

32. The method according to claim 29, wherein the quantified amount of dimeric human AMH in the sample corresponds to a sample concentration in the range from about 1 pg/mL to about 10 pg/mL.

33. A method of quantifying an amount of a pro-mature form of human AMH in a sample, the method comprising performing a sandwich ELISA assay on the sample using a pair of anti-AMH antibodies, wherein one antibody of the pair binds to a first epitope within the pro region of human AMH and the other antibody of the pair binds to a second epitope within the mature region of human AMH, the first epitope contained in an amino acid sequence selected from the group consisting of: SEQ ID NO:106, SEQ ID NO:113, SEQ ID NO:150, SEQ ID NO:132, SEQ ID NO:129, SEQ ID NO:162, SEQ ID NO:163, SEQ ID NO:135, SEQ ID NO:152, SEQ ID NO:153, SEQ ID NO:149, SEQ ID NO:138, and SEQ ID NO:148; and the second epitope contained in an amino acid sequence selected from the group consisting of: SEQ ID NO:173, SEQ ID NO: 169, SEQ ID NO: 170, SEQ ID NO: 168, and SEQ ID NO: 171.

34.-38. (canceled)

39. A method for quantifying human AMH in a sample, the method comprising:
performing a sandwich ELISA on the sample using a capture antibody and a detection antibody, wherein the capture antibody specifically binds to a first epitope of human AMH and the detection antibody binds to a second epitope of human AMH, wherein the first and the second epitopes are contained in same or different amino acid sequences selected from the group consisting of: SEQ ID NO:106, SEQ ID NO:113, SEQ ID NO:150, SEQ ID NO:132, SEQ ID NO:129, SEQ ID NO:162, SEQ ID NO:163, SEQ ID NO:135, SEQ ID NO:152, SEQ ID NO:153, SEQ ID NO:149, SEQ ID NO:138, SEQ ID NO:148, SEQ ID NO:173, SEQ ID NO: 169, SEQ ID NO:170, SEQ ID NO:168, and SEQ ID NO:171.

40.-43. (canceled)

44. A method of quantifying a pro fragment or region of human AMH in a biological sample, the method comprising:
treating a portion of the sample with a detergent under conditions sufficient to dissociate a cleaved-reassociated isoform of human AMH into pro and mature fragments;
quantifying an amount of a pro fragment or region of human AMH by performing a sandwich ELISA on the treated sample using a first antibody for capture and a second antibody for detection, wherein the first and second antibodies specifically bind to the same or different epitopes in the pro portion of human AMH, and wherein the epitopes are contained in an amino acid sequence selected from the group consisting of: SEQ ID NO:106, SEQ ID NO:113, SEQ ID NO:150, SEQ ID NO:132, SEQ ID NO:129, SEQ ID NO:162, SEQ ID NO:163, SEQ ID NO:135, SEQ ID NO:152, SEQ ID NO: 153, SEQ ID NO: 149, SEQ ID NO: 138, and SEQ ID NO: 148;
measuring a detection signal generated by an agent conjugated to the second antibody; and
calculating the amount of pro fragment or region of human AMH in the sample by comparing the detection signal to a calibration curve correlating the amount of pro fragment or region of human AMH to the detection signal.

45. (canceled)

46. A method of quantifying a mature fragment or region of human AMH in a biological sample, the method comprising:
treating a portion of the sample with a detergent under conditions sufficient to dissociate a cleaved-reassociated isoform of human AMH into pro and mature fragments;
quantifying an amount of a mature fragment or region of human AMH by performing a sandwich ELISA on the treated sample using a first antibody for capture and a second antibody for detection, wherein the first and second antibodies specifically bind to the same or different epitopes in the mature portion of human AMH, and wherein the epitopes are contained in an amino acid sequence selected from the group consisting of: SEQ ID NO:173, SEQ ID NO: 169, SEQ ID NO: 170, SEQ ID NO: 168, and SEQ ID NO: 171;
measuring a detection signal generated by an agent conjugated to the second antibody; and
calculating the amount of mature fragment or region of human AMH in the sample by comparing the detection signal to a calibration curve correlating the amount of mature fragment or region of human AMH to the detection signal.

47. (canceled)

48. A method of quantifying an uncleaved pro-mature form of human AMH in a biological sample, the method comprising:
treating a port on of the sample with a detergent under conditions sufficient to dissociate a cleaved-reassociated isoform of human AMH into pro and mature fragments;
quantifying an amount of a pro-mature form of human AMH by performing a sandwich ELISA on the treated sample using a first antibody for capture and a second antibody for detection, wherein the first antibody specifically binds to an epitope in the pro portion of human AMH, wherein the epitope is contained in an amino acid sequence selected from the group consisting of: SEQ ID NO:106, SEQ ID NO:113, SEQ ID NO:150, SEQ ID NO:132, SEQ ID NO:129, SEQ ID NO:162, SEQ ID NO:163, SEQ ID NO:135, SEQ ID NO:152, SEQ ID NO: 153, SEQ ID NO: 149, SEQ ID NO: 138, and SEQ ID NO: 148; and the second antibody specifically binds to an epitope in the mature portion of human AMH, wherein the epitope is contained in an amino acid sequence selected from the group consisting of: SEQ ID NO:173, SEQ ID NO: 169, SEQ ID NO: 170, SEQ ID NO: 168, and SEQ ID NO: 171;
measuring a detection signal generated by an agent conjugated to the second antibody; and
calculating the amount of pro-mature form of human AMH in the sample by comparing the detection signal to a calibration curve correlating the amount of pro-mature form of human AMH to the detection signal.

49. A method of quantifying an uncleaved pro-mature form of human AMH in a biological sample, the method comprising:
treating a portion of the sample with a detergent under conditions sufficient to dissociate a cleaved-reassociated isoform of human AMH into pro and mature fragments;
quantifying an amount of a pro-mature form of human AMH by performing a sandwich ELISA on the treated sample using a first antibody for capture and a second antibody for detection, wherein the first antibody specifically binds to an epitope in the mature portion of human AMH, wherein the epitope is contained in an amino acid sequence selected from the group consisting of: SEQ ID NO:173, SEQ ID NO: 169, SEQ ID NO: 170, SEQ ID NO: 168, and SEQ ID NO: 171; and the second antibody specifically binds to an epitope in the pro portion of human AMH, wherein the epitope is contained in an amino acid sequence selected from the group consisting of: SEQ ID NO:106, SEQ ID NO:113, SEQ ID NO:150, SEQ ID NO:132, SEQ ID NO:129, SEQ ID NO:162, SEQ ID NO:163, SEQ ID NO:135, SEQ ID NO:152, SEQ ID NO: 153, SEQ ID NO: 149, SEQ ID NO: 138, and SEQ ID NO: 148;
measuring a detection signal generated by an agent conjugated to the second antibody; and
calculating the amount of pro-mature form of human AMH in the sample by comparing the detection signal to a calibration curve correlating the amount of pro-mature form of human AMH to the detection signal.

50. A method of quantifying a cleaved-reassociated pro-mature form of human AMH in a biological sample, the method comprising:
(i) performing the method of claim 33 using a first portion of the sample that has not been detergent-treated and in which pro-mature forms of human AMH remain associated, whereby an amount of total (i.e., sum of cleaved and uncleaved pro-mature forms) pro-mature form of human AMH is determined;
(ii) performing the method of claim 48 or of claim 49 using a second portion of the sample, whereby an amount of uncleaved pro-mature form of human AMH is determined; and
(iii) subtracting the amount of uncleaved pro-mature form of human AMH determined in (ii) from the amount of total pro-mature form of human AMH determined in (i) to yield an amount of cleaved-reassociated pro-mature form of human AMH.

51. A method to aid in diagnosing or prognosing a disease or condition selected from the group consisting of: granulosa cell tumors, disorders of sex development, polycystic ovarian syndrome, gonadotoxicity; the method comprising quantifying AMH concentration in a patient sample using the isolated antibody according to claim 1.

52. The method according to claim 51, wherein the disorder of sex development is selected from conditions of newborns with atypical genitalia, conditions of adolescents presenting atypical sexual development, cryptorchidism, and atypical AMH production by the Sertoli cells of testes and its effects.

53. The method according to claim 51, wherein the gonadotoxicity is induced by chemotherapy.

54. A method of determining ovarian reserve in a female human subject, the method comprising quantifying AMH in a biological sample from the subject using the isolated antibody according to claim 1.

55.-56. (canceled)

57. A method to aid in diagnosing ovarian insufficiency, the method comprising quantifying AMH in a biological sample using the isolated antibody according to claim 1.

58. A method of predicting time to menopause in a female human subject, the method comprising quantifying AMH in a sample from the subject using the isolated antibody according to claim 1.

59.-60. (canceled)

61. A method to aid in diagnosing or prognosing an AMH-related disease or condition, the method comprising quantifying AMH concentration in a patient sample using the isolated antibody according to claim 1, wherein the AMH-related disease or condition is selected from the group consisting of: peri-menopausal transition, neonatal gender, cryptorchidism, testicular function, and precocious puberty.

62. An ELISA kit for quantification of human AMH, the kit comprising a pair of anti-AMH antibodies, the pair consisting of a capture antibody and a detection antibody, and instructions for use; wherein the capture antibody and the detection antibody bind to a first and a second epitope, respectively; and wherein each of the first and the second epitopes is contained in an amino acid sequence selected from the group consisting of: SEQ ID NO:106, SEQ ID NO:113, SEQ ID NO:150, SEQ ID NO:132, SEQ ID NO:129, SEQ ID NO:162, SEQ ID NO:163, SEQ ID NO:135, SEQ ID NO:152, SEQ ID NO:153, SEQ ID NO:149, SEQ ID NO:138, SEQ ID NO:148, SEQ ID NO:173, SEQ ID NO:169, SEQ ID NO:170, SEQ ID NO:168, and SEQ ID NO:171.

63.-68. (canceled)

69. The kit according to claim 62, which is adapted for determination of human AMH extracted from a dried blood sample.

70. The kit according to claim 69 wherein the dried blood sample is in the form of dried blood adsorbed to a disc of an adsorbent material.

71. A method of quantifying a cleaved-reassociated pro-mature form of human AMH in a biological sample, the method comprising:
(i) performing the method of claim 44 or claim 46, whereby an amount of total (i.e., sum of cleaved-reassociated and uncleaved pro-mature forms) pro-mature form of human AMH is determined;
(ii) performing the method of claim 48 or claim 49, whereby an amount of uncleaved pro-mature form of human AMH is determined; and
(iii) subtracting the amount of uncleaved pro-mature form of human AMH determined in (ii) from the amount of total pro-mature form of human AMH determined in (i) to yield an amount of cleaved-reassociated pro-mature form of human AMH.

72. A method of quantifying a fraction of uncleaved pro-mature form of human AMH in a biological sample, the method comprising:
(i) performing the method of claim 44 or claim 46, whereby an amount of total (i.e., sum of cleaved-reassociated and uncleaved pro-mature forms) pro-mature form of human AMH is determined;
(ii) performing the method of claim 48 or claim 49, whereby an amount of uncleaved pro-mature form of human AMH is determined; and
(iii) determining a ratio of the amount determined in (ii) to the amount determined in (i) as a measure of the fraction of uncleaved pro-mature form of human AMH in the sample.


SEQUENCE LISTING (180 sequences)

SEQ ID NO: 1 - DNA, 393 bp, Homo sapiens
atggtgtccacttctcagctccttggacttttgcttttctggacttcagcctccagatgtgacattatgatgactcagtctccagccaccctgtctgtgactccaggagatagagtctctctttcctgcagggccagccagagtattagcgactacttacactggtatcaacaaaaatcacatgagtctccaaggcttctcatcaaatatgcttcccaatccatctctgggatcccctccaggttcagtggcagtggatcagggtcagatttcactctcagtatcaacagtgtggaacctgaagatgttggagtgtattactgtcaaaatggtcacagctttccgctcacgttcggagctgggaccaagctggagctgaaacgggctgatgct

SEQ ID NO: 2 - protein, 131 aa, Homo sapiens
MVSTSQLLGLLLFWTSASRCDIMMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKRADA

SEQ ID NO: 3 - protein, 11 aa, Homo sapiens
RASQSISDYLH

SEQ ID NO: 4 - protein, 7 aa, Homo sapiens
YASQSIS

SEQ ID NO: 5 - protein, 9 aa, Homo sapiens
QNGHSFPLT

SEQ ID NO: 6 - DNA, 408 bp, Homo sapiens
atggaatggagctgggtctctctcttcttcctgtcagtaactacaggtgtccactcccaggttcagctgcaacagtctgacgctgagttggtgaaacctggagcttcagtgaagatatcctgcaaggtttctggctacaccttcactgaccatacttttcactggatgaagcagaggcctgaacagggcctggaatggattggatatatttatcctagagatggtagtactgattacaatgagaagttcaagggcaaggccacattgactgcagacagatcctccagcacagtctacatgcagctcaacagcctgacatctgaggactctgcagtttatttctgtgcaagagatctcaggctttctttttcttactggggccaagggactctggtcactgtctctgca

SEQ ID NO: 7 - protein, 136 aa, Homo sapiens
MEWSWVSLFFLSVTTGVHSQVQLQQSDAELVKPGASVKISCKVSGYTFTDHTFHWMKQRPEQGLEWIGYIYPRDGSTDYNEKFKGKATLTADRSSSTVYMQLNSLTSEDSAVYFCARDLRLSFSYWGQGTLVTVSA

SEQ ID NO: 8 - protein, 5 aa, Homo sapiens
DHTFH

SEQ ID NO: 9 - protein, 17 aa, Homo sapiens
YIYPRDGSTDYNEKFKG

SEQ ID NO: 10 - protein, 8 aa, Homo sapiens
DLRLSFSY

SEQ ID NO: 11 - DNA, 393 bp, Homo sapiens
atgatgtcctctgctcagttccttggtctcctgttgctctgttttcaaggtaccagatgtgatatccagatgacacagactacatcctccctgtctgcctctctgggagacagagtcaccatcagttgcagggcaagtcaggacattagcaattatttaaactggtatcagcagaaaccagatggaactgttaaactcctgatttactacacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggaaataaaacgggctgatgct

SEQ ID NO: 12 - protein, 129 aa, Homo sapiens
MMSSAQFLGLLLLCFQGTRCDIQMTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIKRA

SEQ ID NO: 13 - protein, 11 aa, Homo sapiens
RASQDISNYLN

SEQ ID NO: 14 - protein, 7 aa, Homo sapiens
YTSRLHS

SEQ ID NO: 15 - protein, 9 aa, Homo sapiens
QQGNTLPYT

SEQ ID NO: 16 - DNA, 405 bp, Homo sapiens
atggaatggagctgggtctctctcttcttcctgtcagtaactacaggtgtccactcccaggttcagctgcaacagtctgacgctgagtcggtgaaaccaggagcttcagtgaagatatcctgcaaggtttctggctacaccttcactgaccatactattcactggatgaagcagaggcctgaacagggcctggaatggattggatatatttatcctagagatggtagtactaactacaatgagaagttcaagggcaaggccacattgactgcagacaaatcctccagcacagcctacatgcagctcaacagcctgacatctgaggactctgcagtctatttctgtgcaagatcatatttggatatggactactggggtcaaggaacctcagtcaccgtctcctca

SEQ ID NO: 17 - protein, 135 aa, Homo sapiens
MEWSWVSLFFLSVTTGVHSQVQLQQSDAESVKPGASVKISCKVSGYTFTDHTIHWMKQRPEQGLEWIGYIYPRDGSTNYNEKFKGKATLTADKSSSTAYMQLNSLTSEDSAVYFCARSYLDMDYWGQGTSVTVSS

SEQ ID NO: 18 - protein, 5 aa, Homo sapiens
DHTIH

SEQ ID NO: 19 - protein, 17 aa, Homo sapiens
YIYPRDGSTNYNEKFKG

SEQ ID NO: 20 - protein, 7 aa, Homo sapiens
SYLDMDY

SEQ ID NO: 21 - protein, 16 aa, Artificial Sequence
  Synthetic peptide containing a region of human AMH protein
SGSGMRDLPLTSLALV

SEQ ID NO: 22 - protein, 16 aa, Artificial Sequence
  Synthetic peptide containing a region of human AMH protein
SGSGSLALVLSALGAL

SEQ ID NO: 23 - protein, 16 aa, Artificial Sequence
  Synthetic peptide containing a region of human AMH protein
SGSGALGALLGTEALR

SEQ ID NO: 24 - protein, 16 aa, Artificial Sequence
  Synthetic peptide containing a region of human AMH protein
SGSGTEALRAEEPAVG

SEQ ID NO: 25 - protein, 16 aa, Artificial Sequence
  Synthetic peptide containing a region of human AMH protein
SGSGEPAVGTSGLIFR

SEQ ID NO: 26 - protein, 16 aa, Artificial Sequence
  Synthetic peptide containing a region of human AMH protein
SGSGGLIFREDLDWPP

SEQ ID NO: 27 - protein, 16 aa, Artificial Sequence
  Synthetic peptide containing a region of human AMH protein
SGSGLDWPPGIPQEPL

SEQ ID NO: 28 - protein, 16 aa, Artificial Sequence
  Synthetic peptide containing a region of human AMH protein
SGSGPQEPLCLVALGG

SEQ ID NO: 29 - protein, 16 aa, Artificial Sequence
  Synthetic peptide containing a region of human AMH protein
SGSGVALGGDSNGSSS

SEQ ID NO: 30 - protein, 16 aa, Artificial Sequence
  Synthetic peptide containing a region of human AMH protein
SGSGNGSSSPLRVVGA

SEQ ID NO: 31 - protein, 16 aa, Artificial Sequence
  Synthetic peptide containing a region of human AMH protein
SGSGRVVGALSAYEQA

SEQ ID NO: 32 - protein, 16 aa, Artificial Sequence
  Synthetic peptide containing a region of human AMH protein
SGSGAYEQAFLGAVQR

SEQ ID NO: 33 - protein, 16 aa, Artificial Sequence
  Synthetic peptide containing a region of human AMH protein
SGSGGAVQRARWGPRD

SEQ ID NO: 34 - protein, 16 aa, Artificial Sequence
  Synthetic peptide containing a region of human AMH protein
SGSGWGPRDLATFGVC

SEQ ID NO: 35 - protein, 16 aa, Artificial Sequence
  Synthetic peptide containing a region of human AMH protein
SGSGTFGVCNTGDRQA

SEQ ID NO: 36 - protein, 16 aa, Artificial Sequence
  Synthetic peptide containing a region of human AMH protein
SGSGGDRQAALPSLRR

SEQ ID NO: 37 - protein, 16 aa, Artificial Sequence
  Synthetic peptide containing a region of human AMH protein
SGSGPSLRRLGAWLRD

SEQ ID NO: 38 - protein, 16 aa, Artificial Sequence
  Synthetic peptide containing a region of human AMH protein
SGSGAWLRDPGGQRLV

SEQ ID NO: 39 - protein, 16 aa, Artificial Sequence
  Synthetic peptide containing a region of human AMH protein
SGSGGQRLVVLHLEEV

SEQ ID NO: 40 - protein, 16 aa, Artificial Sequence
  Synthetic peptide containing a region of human AMH protein
SGSGHLEEVTWEPTPS

SEQ ID NO: 41 - protein, 16 aa, Artificial Sequence
  Synthetic peptide containing a region of human AMH protein
SGSGEPTPSLRFQEPP

SEQ ID NO: 42 - protein, 16 aa, Artificial Sequence
  Synthetic peptide containing a region of human AMH protein
SGSGFQEPPPGGAGPP

SEQ ID NO: 43 - protein, 16 aa, Artificial Sequence
  Synthetic peptide containing a region of human AMH protein
SGSGGAGPPELALLVL

SEQ ID NO: 44 - protein, 16 aa, Artificial Sequence
  Synthetic peptide containing a region of human AMH protein
SGSGALLVLYPGPGPE

SEQ ID NO: 45 - protein, 16 aa, Artificial Sequence
  Synthetic peptide containing a region of human AMH protein
SGSGGPGPEVTVTRAG

SEQ ID NO: 46 - protein, 16 aa, Artificial Sequence
  Synthetic peptide containing a region of human AMH protein
SGSGVTRAGLPGAQSL

SEQ ID NO: 47 - protein, 16 aa, Artificial Sequence
  Synthetic peptide containing a region of human AMH protein
SGSGGAQSLCPSRDTR

SEQ ID NO: 48 - protein, 16 aa, Artificial Sequence
  Synthetic peptide containing a region of human AMH protein
SGSGSRDTRYLVLAVD

SEQ ID NO: 49 - protein, 16 aa, Artificial Sequence
  Synthetic peptide containing a region of human AMH protein
SGSGVLAVDRPAGAWR

SEQ ID NO: 50 - protein, 16 aa, Artificial Sequence
  Synthetic peptide containing a region of human AMH protein
SGSGAGAWRGSGLALT

SEQ ID NO: 51 - protein, 16 aa, Artificial Sequence
  Synthetic peptide containing a region of human AMH protein
SGSGGLALTLQPRGED

SEQ ID NO: 52 - protein, 16 aa, Artificial Sequence
  Synthetic peptide containing a region of human AMH protein
SGSGPRGEDSRLSTAR

SEQ ID NO: 53 - protein, 16 aa, Artificial Sequence
  Synthetic peptide containing a region of human AMH protein
SGSGLSTARLQALLFG

SEQ ID NO: 54 - protein, 16 aa, Artificial Sequence
  Synthetic peptide containing a region of human AMH protein
SGSGALLFGDDHRCFT

SEQ ID NO: 55 - protein, 16 aa, Artificial Sequence
  Synthetic peptide containing a region of human AMH protein
SGSGHRCFTRMTPALL

SEQ ID NO: 56 - protein, 16 aa, Artificial Sequence
  Synthetic peptide containing a region of human AMH protein
SGSGTPALLLLPRSEP

SEQ ID NO: 57 - protein, 16 aa, Artificial Sequence
  Synthetic peptide containing a region of human AMH protein
SGSGPRSEPAPLPAHG

SEQ ID NO: 58 - protein, 16 aa, Artificial Sequence
  Synthetic peptide containing a region of human AMH protein
SGSGLPAHGQLDTVPF

SEQ ID NO: 59 - protein, 16 aa, Artificial Sequence
  Synthetic peptide containing a region of human AMH protein
SGSGDTVPFPPPRPSA

SEQ ID NO: 60 - protein, 16 aa, Artificial Sequence
  Synthetic peptide containing a region of human AMH protein
SGSGPRPSAELEESPP

SEQ ID NO: 61 - protein, 16 aa, Artificial Sequence
  Synthetic peptide containing a region of human AMH protein
SGSGEESPPSADPFLE

SEQ ID NO: 62 - protein, 16 aa, Artificial Sequence
  Synthetic peptide containing a region of human AMH protein
SGSGDPFLETLTRLVR

SEQ ID NO: 63 - protein, 16 aa, Artificial Sequence
  Synthetic peptide containing a region of human AMH protein
SGSGTRLVRALRVPPA

SEQ ID NO: 64 - protein, 16 aa, Artificial Sequence
  Synthetic peptide containing a region of human AMH protein
SGSGRVPPARASAPRL

SEQ ID NO: 65 - protein, 16 aa, Artificial Sequence
  Synthetic peptide containing a region of human AMH protein
SGSGSAPRLALDPDAL

SEQ ID NO: 66 - protein, 16 aa, Artificial Sequence
  Synthetic peptide containing a region of human AMH protein
SGSGDPDALAGFPQGL

SEQ ID NO: 67 - protein, 16 aa, Artificial Sequence
  Synthetic peptide containing a region of human AMH protein
SGSGFPQGLVNLSDPA

SEQ ID NO: 68 - protein, 16 aa, Artificial Sequence
  Synthetic peptide containing a region of human AMH protein
SGSGLSDPAALERLLD

SEQ ID NO: 69 - protein, 16 aa, Artificial Sequence
  Synthetic peptide containing a region of human AMH protein
SGSGERLLDGEEPLLL

SEQ ID NO: 70 - protein, 16 aa, Artificial Sequence
  Synthetic peptide containing a region of human AMH protein
SGSGEPLLLLLRPTAA

SEQ ID NO: 71 - protein, 16 aa, Artificial Sequence
  Synthetic peptide containing a region of human AMH protein
SGSGRPTAATTGDPAP

SEQ ID NO: 72 - protein, 16 aa, Artificial Sequence
  Synthetic peptide containing a region of human AMH protein
SGSGGDPAPLHDPTSA

SEQ ID NO: 73 - protein, 16 aa, Artificial Sequence
  Synthetic peptide containing a region of human AMH protein
SGSGDPTSAPWATALA

SEQ ID NO: 74 - protein, 16 aa, Artificial Sequence
  Synthetic peptide containing a region of human AMH protein
SGSGATALARRVAAEL

SEQ ID NO: 75 - protein, 16 aa, Artificial Sequence
  Synthetic peptide containing a region of human AMH protein
SGSGVAAELQAAAAEL

SEQ ID NO: 76 - protein, 16 aa, Artificial Sequence
  Synthetic peptide containing a region of human AMH protein
SGSGAAAELRSLPGLP

SEQ ID NO: 77 - protein, 16 aa, Artificial Sequence
  Synthetic peptide containing a region of human AMH protein
SGSGLPGLPPATAPLL

SEQ ID NO: 78 - protein, 16 aa, Artificial Sequence
  Synthetic peptide containing a region of human AMH protein
SGSGTAPLLARLLALC

SEQ ID NO: 79 - protein, 16 aa, Artificial Sequence
  Synthetic peptide containing a region of human AMH protein
SGSGLLALCPGGPGGL

SEQ ID NO: 80 - protein, 16 aa, Artificial Sequence
  Synthetic peptide containing a region of human AMH protein
SGSGGPGGLGDPLRAL

SEQ ID NO: 81 - protein, 16 aa, Artificial Sequence
  Synthetic peptide containing a region of human AMH protein
SGSGPLRALLLLKALQ

SEQ ID NO: 82 - protein, 16 aa, Artificial Sequence
  Synthetic peptide containing a region of human AMH protein
SGSGLKALQGLRVEWR

SEQ ID NO: 83 - protein, 16 aa, Artificial Sequence
  Synthetic peptide containing a region of human AMH protein
SGSGRVEWRGRDPRGP

SEQ ID NO: 84 - protein, 16 aa, Artificial Sequence
  Synthetic peptide containing a region of human AMH protein
SGSGDPRGPGRAQRSA

SEQ ID NO: 85 - protein, 16 aa, Artificial Sequence
  Synthetic peptide containing a region of human AMH protein
SGSGAQRSAGATAADG

SEQ ID NO: 86 - protein, 16 aa, Artificial Sequence
  Synthetic peptide containing a region of human AMH protein
SGSGTAADGPCALREL

SEQ ID NO: 87 - protein, 16 aa, Artificial Sequence
  Synthetic peptide containing a region of human AMH protein
SGSGALRELSVDLRAE

SEQ ID NO: 88 - protein, 16 aa, Artificial Sequence
  Synthetic peptide containing a region of human AMH protein
SGSGDLRAERSVLIPE

SEQ ID NO: 89 - protein, 16 aa, Artificial Sequence
  Synthetic peptide containing a region of human AMH protein
SGSGVLIPETYQANNC

SEQ ID NO: 90 - protein, 16 aa, Artificial Sequence
  Synthetic peptide containing a region of human AMH protein
SGSGQANNCQGVCGWP

SEQ ID NO: 91 - protein, 16 aa, Artificial Sequence
  Synthetic peptide containing a region of human AMH protein
SGSGVCGWPQSDRNPR

SEQ ID NO: 92 - protein, 16 aa, Artificial Sequence
  Synthetic peptide containing a region of human AMH protein
SGSGDRNPRYGNHVVL

SEQ ID NO: 93 - protein, 16 aa, Artificial Sequence
  Synthetic peptide containing a region of human AMH protein
SGSGNHVVLLLKMQVR

SEQ ID NO: 94 - protein, 16 aa, Artificial Sequence
  Synthetic peptide containing a region of human AMH protein
SGSGKMQVRGAALARP

SEQ ID NO: 95 - protein, 16 aa, Artificial Sequence
  Synthetic peptide containing a region of human AMH protein
SGSGALARPPCCVPTA

SEQ ID NO: 96 - protein, 16 aa, Artificial Sequence
  Synthetic peptide containing a region of human AMH protein
SGSGCVPTAYAGKLLI

SEQ ID NO: 97 - protein, 16 aa, Artificial Sequence
  Synthetic peptide containing a region of human AMH protein
SGSGGKLLISLSEERI

SEQ ID NO: 98 - protein, 16 aa, Artificial Sequence
  Synthetic peptide containing a region of human AMH protein
SGSGSEERISAHHVPN

SEQ ID NO: 99 - protein, 16 aa, Artificial Sequence
  Synthetic peptide containing a region of human AMH protein
SGSGHHVPNMVATECG

SEQ ID NO: 100 - protein, 16 aa, Artificial Sequence
  Synthetic peptide containing a region of human AMH protein
SGSGVPNMVATECGCR

SEQ ID NO: 101 - protein, 12 aa, Homo sapiens
MRDLPLTSLALV

SEQ ID NO: 102 - protein, 12 aa, Homo sapiens
SLALVLSALGAL

SEQ ID NO: 103 - protein, 12 aa, Homo sapiens
ALGALLGTEALR

SEQ ID NO: 104 - protein, 12 aa, Homo sapiens
TEALRAEEPAVG

SEQ ID NO: 105 - protein, 12 aa, Homo sapiens
EPAVGTSGLIFR

SEQ ID NO: 106 - protein, 12 aa, Homo sapiens
GLIFREDLDWPP

SEQ ID NO: 107 - protein, 12 aa, Homo sapiens
LDWPPGIPQEPL

SEQ ID NO: 108 - protein, 12 aa, Homo sapiens
PQEPLCLVALGG

SEQ ID NO: 109 - protein, 12 aa, Homo sapiens
VALGGDSNGSSS

SEQ ID NO: 110 - protein, 12 aa, Homo sapiens
NGSSSPLRVVGA

SEQ ID NO: 111 - protein, 12 aa, Homo sapiens
RVVGALSAYEQA

SEQ ID NO: 112 - protein, 12 aa, Homo sapiens
AYEQAFLGAVQR

SEQ ID NO: 113 - protein, 12 aa, Homo sapiens
GAVQRARWGPRD

SEQ ID NO: 114 - protein, 12 aa, Homo sapiens
WGPRDLATFGVC

SEQ ID NO: 115 - protein, 12 aa, Homo sapiens
TFGVCNTGDRQA

SEQ ID NO: 116 - protein, 12 aa, Homo sapiens
GDRQAALPSLRR

SEQ ID NO: 117 - protein, 12 aa, Homo sapiens
PSLRRLGAWLRD

SEQ ID NO: 118 - protein, 12 aa, Homo sapiens
AWLRDPGGQRLV

SEQ ID NO: 119 - protein, 12 aa, Homo sapiens
GQRLVVLHLEEV

SEQ ID NO: 120 - protein, 12 aa, Homo sapiens
HLEEVTWEPTPS

SEQ ID NO: 121 - protein, 12 aa, Homo sapiens
EPTPSLRFQEPP

SEQ ID NO: 122 - protein, 12 aa, Homo sapiens
FQEPPPGGAGPP

SEQ ID NO: 123 - protein, 12 aa, Homo sapiens
GAGPPELALLVL

SEQ ID NO: 124 - protein, 12 aa, Homo sapiens
ALLVLYPGPGPE

SEQ ID NO: 125 - protein, 12 aa, Homo sapiens
GPGPEVTVTRAG

SEQ ID NO: 126 - protein, 12 aa, Homo sapiens
VTRAGLPGAQSL

SEQ ID NO: 127 - protein, 12 aa, Homo sapiens
GAQSLCPSRDTR

SEQ ID NO: 128 - protein, 12 aa, Homo sapiens
SRDTRYLVLAVD

SEQ ID NO: 129 - protein, 12 aa, Homo sapiens
VLAVDRPAGAWR

SEQ ID NO: 130 - protein, 12 aa, Homo sapiens
AGAWRGSGLALT

SEQ ID NO: 131 - protein, 12 aa, Homo sapiens
GLALTLQPRGED

SEQ ID NO: 132 - protein, 12 aa, Homo sapiens
PRGEDSRLSTAR

SEQ ID NO: 133 - protein, 12 aa, Homo sapiens
LSTARLQALLFG

SEQ ID NO: 134 - protein, 12 aa, Homo sapiens
ALLFGDDHRCFT

SEQ ID NO: 135 - protein, 12 aa, Homo sapiens
HRCFTRMTPALL

SEQ ID NO: 136 - protein, 12 aa, Homo sapiens
TPALLLLPRSEP

SEQ ID NO: 137 - protein, 12 aa, Homo sapiens
PRSEPAPLPAHG

SEQ ID NO: 138 - protein, 12 aa, Homo sapiens
LPAHGQLDTVPF

SEQ ID NO: 139 - protein, 12 aa, Homo sapiens
DTVPFPPPRPSA

SEQ ID NO: 140 - protein, 12 aa, Homo sapiens
PRPSAELEESPP

SEQ ID NO: 141 - protein, 12 aa, Homo sapiens
EESPPSADPFLE

SEQ ID NO: 142 - protein, 12 aa, Homo sapiens
DPFLETLTRLVR

SEQ ID NO: 143 - protein, 12 aa, Homo sapiens
TRLVRALRVPPA

SEQ ID NO: 144 - protein, 12 aa, Homo sapiens
RVPPARASAPRL

SEQ ID NO: 145 - protein, 12 aa, Homo sapiens
SAPRLALDPDAL

SEQ ID NO: 146 - protein, 12 aa, Homo sapiens
DPDALAGFPQGL

SEQ ID NO: 147 - protein, 12 aa, Homo sapiens
FPQGLVNLSDPA

SEQ ID NO: 148 - protein, 12 aa, Homo sapiens
LSDPAALERLLD

SEQ ID NO: 149 - protein, 12 aa, Homo sapiens
ERLLDGEEPLLL

SEQ ID NO: 150 - protein, 12 aa, Homo sapiens
EPLLLLLRPTAA

SEQ ID NO: 151 - protein, 12 aa, Homo sapiens
RPTAATTGDPAP

SEQ ID NO: 152 - protein, 12 aa, Homo sapiens
GDPAPLHDPTSA

SEQ ID NO: 153 - protein, 12 aa, Homo sapiens
DPTSAPWATALA

SEQ ID NO: 154 - protein, 12 aa, Homo sapiens
ATALARRVAAEL

SEQ ID NO: 155 - protein, 12 aa, Homo sapiens
VAAELQAAAAEL

SEQ ID NO: 156 - protein, 12 aa, Homo sapiens
AAAELRSLPGLP

SEQ ID NO: 157 - protein, 12 aa, Homo sapiens
LPGLPPATAPLL

SEQ ID NO: 158 - protein, 12 aa, Homo sapiens
TAPLLARLLALC

SEQ ID NO: 159 - protein, 12 aa, Homo sapiens
LLALCPGGPGGL

SEQ ID NO: 160 - protein, 12 aa, Homo sapiens
GPGGLGDPLRAL

SEQ ID NO: 161 - protein, 12 aa, Homo sapiens
PLRALLLLKALQ

SEQ ID NO: 162 - protein, 12 aa, Homo sapiens
LKALQGLRVEWR

SEQ ID NO: 163 - protein, 12 aa, Homo sapiens
RVEWRGRDPRGP

SEQ ID NO: 164 - protein, 12 aa, Homo sapiens
DPRGPGRAQRSA

SEQ ID NO: 165 - protein, 12 aa, Homo sapiens
AQRSAGATAADG

SEQ ID NO: 166 - protein, 12 aa, Homo sapiens
TAADGPCALREL

SEQ ID NO: 167 - protein, 12 aa, Homo sapiens
ALRELSVDLRAE

SEQ ID NO: 168 - protein, 12 aa, Homo sapiens
DLRAERSVLIPE

SEQ ID NO: 169 - protein, 12 aa, Homo sapiens
VLIPETYQANNC

SEQ ID NO: 170 - protein, 12 aa, Homo sapiens
QANNCQGVCGWP

SEQ ID NO: 171 - protein, 12 aa, Homo sapiens
VCGWPQSDRNPR

SEQ ID NO: 172 - protein, 12 aa, Homo sapiens
DRNPRYGNHVVL

SEQ ID NO: 173 - protein, 12 aa, Homo sapiens
NHVVLLLKMQVR

SEQ ID NO: 174 - protein, 12 aa, Homo sapiens
KMQVRGAALARP

SEQ ID NO: 175 - protein, 12 aa, Homo sapiens
ALARPPCCVPTA

SEQ ID NO: 176 - protein, 12 aa, Homo sapiens
CVPTAYAGKLLI

SEQ ID NO: 177 - protein, 12 aa, Homo sapiens
GKLLISLSEERI

SEQ ID NO: 178 - protein, 12 aa, Homo sapiens
SEERISAHHVPN

SEQ ID NO: 179 - protein, 12 aa, Homo sapiens
HHVPNMVATECG

SEQ ID NO: 180 - protein, 12 aa, Homo sapiens
VPNMVATECGCR